(19) United States
(12) Patent Application Publication
(10) Pub. No.: US 2002/0013958 A1
(43) Pub. Date: Jan. 31, 2002

(54) Polynucleotides and polypeptides derived from corn seedling

(75) Inventors: Raghunath V. Lalgudi, Clayton, MO (US); Laura Y. Ito, Pleasanton, CA (US); Bradley K. Sherman, Oakland, CA (US)

Correspondence Address: INCYTE GENOMICS, INC., PATENT DEPARTMENT, 3160 Porter Drive, Palo Alto, CA, 94304, US

(73) Assignee: Incyte Pharmaceuticals, Inc.

(21) Appl. No.: 09/923,876

(22) Filed: Aug. 6, 2001

Related U.S. Application Data
(63) Continuation of application No. 09/298,329, filed on Apr. 21, 1999.
(60) Provisional application No. 60/085,331, filed on May 12, 1998.

(51) Int. Cl. A01H 5/00; C12N 15/82; C12N 5/14; C12N 15/29

(52) U.S. Cl. 800/298; 536/23.6; 435/6; 435/91.2; 536/24.1; 435/320.1; 435/419; 435/468

(57) ABSTRACT

The present invention provides purified, corn seedling-derived polynucleotides (cdps) which encode corn seedling-derived polypeptides (CDPs). The invention also provides for the use of cdps or their complements, oligonucleotides, or fragments in methods for determining altered gene expression, to recover regulatory elements, and to follow inheritance of desirable characteristics through hybrid breeding programs. The invention further provides for vectors and host cells containing cdps for the expression of CDPs. The invention additionally provides for (i) use of isolated and purified CDPs to induce antibodies and to screen libraries of compounds and (ii) use of anti-CDP antibodies in diagnostic assays.

This application claims the benefit of U.S. application Ser. No. 09/298,329, our Docket No. PL-0012 US, filed Apr. 21, 1999, which claimed the benefit of U.S. Provisional Application No. 60/085,331, our Docket No. PL-0012 P, filed on May 12, 1998.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### DESCRIPTION OF THE COMPACT DISK-RECORDABLE (CD-R)

CD-R 1 contains Tables 1 and 2 formatted in tab-delimited ASCII text, the Sequence Listing formatted in plain ASCII text. CD-R 1 is labeled with Identification No. PL-0012-1 CON, Copy 1. The file containing Table 1 is entitled PL0012-2.TXT, created on Aug. 6, 2001, and is 1,001 KB in size. The file containing Table 2 is entitled PL0012-3.TXT, created on Aug. 6, 2001, and is 128 KB in size. The Sequence Listing is entitled PL-0012-1.TXT, created on Aug. 6, 2001, and is 3,031 KB in size.

CD-R 2 is an exact copy of CD-R 1. CD-R 2 is labeled with Identification No. PL-00012-1 CON, Copy 2.

CD-R 3 contains the Computer Readable Form of the Sequence Listing in compliance with 37 C.F.R. §1.821(e), and specified by 37 C.F.R. §1.824. CD-R 3 is labeled with Identification No. PL-0012-1 CON, Copy 3. The file containing the Sequence Listing is entitled P10012-1, created on Aug. 6, 2001, and is 3,031 KB in size.

The disclosure of Table 1, Table 2, and the Sequence Listing submitted as an electronic document on compact disc as described above are to be part of the permanent USPTO record of this patent application and are hereby expressly incorporated by reference.

### FIELD OF THE INVENTION

The present invention relates to nucleic and amino acid sequences derived from corn seedling and to the use of these sequences in the identification, evaluation, and alteration of desired characteristics associated with growth and development, disease resistance, environmental adaptability, quality, and yield.

### BACKGROUND OF THE INVENTION

The field of plant breeding deals with the manipulation of plant genomes with the purpose of improving characteristics of the plant. Plant breeders use data and methodology from plant physiology, genetics, biochemistry, pathology, statistics, and molecular biology. One of the most improved hybrid crops is corn, Zea mays (L.). Corn is presently the second-most economically important crop in the United States. Acreage of field corn (used for livestock feed, corn starch, corn syrup, fuel ethanol, and oil) and sweet corn (used fresh or processed for human consumption) exceeds that of any other agronomic crop. Annual losses, reduction in quality and yield, due to diseases and infestation may range from 7 to 17%. Studies of corn may be used as a model for other economically important agronomic grasses.

Corn is a monocotyledonous plant which has one seed leaf, uses the C₄ photosynthetic pathway, and has scattered vascular bundles. The mature plant is made up of roots, stem, leaves, and reproductive structures. The root system functions to anchor the plant and to absorb water and nutrients. The corn stem consists of a series of nodes, each bearing one bladelike leaf with parallel veins, and internodes, elongated stem sections. The stem and its leaves are commonly referred to as a stalk. Leaves arise alternately and are arranged in two rows along either side of the stem. The male reproductive structure is the tassel, and its flowers produce pollen. The female reproductive structure is the ear, and its flowers each have a silk for pollination. When a pollen grain is shed onto a silk and germinates, a pollen tube grows down the silk and delivers two sperm to the female gametophyte. Within the gametophyte, one sperm fertilizes the egg, and the other, two polar nuclei. The embryo and endosperm produced by this double fertilization develop into the seed which matures in about two months.

The vegetative (V) and reproductive (R) stages of growth for a corn plant are as follows: VE—emergence from the soil of the seedling leaf; V1—first true leaf; V2—second leaf; . . . V(n)—nth leaf; VT—tasseling stage; and reproductive stages, R1—silking; R2—blister; R3—milk; R4—dough; R5—dent; and R6—physiological maturity (Ritchie, S. W. et al. (1986) How a Corn Plant Develops, Iowa State University Cooperative Extension Service, Ames IA 48:1-21).

Corn Seedling

A seedling encompasses the growth stages from the beginning of germination to the emergence of the first true leaf at V1. Three important environmental factors essential for seed germination are water, necessary for metabolic activity; oxygen, necessary for aerobic respiration; and acceptable temperature, ≧13° C. The coleorhiza, a sheathlike structure, grows through the mature ovary wall or pericarp, followed by the radicle, the primary root, which elongates and penetrates the coleorhiza. In addition to the radicle, three or four lateral, seminal roots, which arise from the cotyledonary node, grow through the pericarp then bend downward. The single cotyledon (seed leaf), the scutellum, remains permanently attached to the endosperm (the embryo's food storage tissue) within the grain. The young primary shoot (plumule) of the seedling is at first enclosed within a tubular ensheathing leaf, the coleoptile. After the primary root emerges, the coleoptile is pushed upward to the soil surface by elongation of the first stem internode (VE). The embryonic leaves soon emerge (V1), develop chlorophyll, and begin photosynthesis. The coleoptile persists for a time at the base of the shoot. (Foster, A. S. and E. M. Gifford, Jr. (1959) Comparative Morphology of Vascular Plants, W. H. Freeman & Co., San Francisco Calif., 555 pp.; Raven, P. et al. (1981) Biology of Plants, Worth Publishers Inc New York N.Y., 686 pp.).

Problems Of Corn Seedlings

All parts of the corn plant are susceptible to diseases, insect infestations, and stress. These conditions are usually diagnosed by their above-ground leaf, stock, fruit and/or seed symptoms and are caused by fungi, bacteria, mycoplasmas and related organisms, viruses, nematodes, parasitic seed plants, insects and mites, and abnormal environmental conditions.

Fungal diseases are spread by spores that germinate under favorable conditions of temperature and moisture. Spores germinate to produce branched threads called hyphae that infect plants by direct penetration of the epidermis or through natural openings or wounds. Resting bodies (chlamydospores, sclerotia) allow fungi to survive under unfavorable conditions for long periods in the soil or in plant debris.

Germinating seed and seedlings may be attacked by a number of soilborne or seedborne fungi that cause seed rots, seedling wilt, and seedling blights. The fungi responsible include Fusarium spp., Pythium spp., Diplodia spp., Bipolaris maydis strain T, and Penicillium spp. Aboveground symptoms include yellowing, wilting and death of the leaves; rotting of seed before or during germination; soft rot of the culm near the ground and water-soaking of seedling tissues. The rotted area may be of different colors depending on the infecting fungal species. Seedling wilt symptoms include a gray color at the leaf tips extending rapidly throughout the whole leaf which result in complete collapse and death of the seedling within two days.

Plant-pathogenic bacteria are unicellular non-spore-forming rods with or without flagella which are spread by cultural practices, animals, water, and soil. Bacteria enter plants through wounds or natural openings and multiply rapidly inside the plant where they cause death of cells; abnormal growths; block water-conducting tissue; or break down the tissue structure. They can remain dormant on or within plant tissue, insects, soil, or equipment.

Goss' Bacterial Wilt and Blight caused by Clavibacter michiganensis ssp. nebraskensis, affects corn in the Midwest with seedlings more susceptible than more mature plants. In susceptible corn varieties, lesions cause the seedlings to wilt, wither, and die. C. michiganensis ssp. nebraskensis overwinters in infected corn debris left on the soil surface and in infected seed. Other important bacterial diseases affection corn seedlings include Stewart's Bacterial Wilt; Holcus Spot; Bacterial Leaf Blight; and Bacterial Stripe and Leaf Spot. Viruses are submicroscopic particles composed of nucleic acid and protein which are transmitted by biological vectors, e.g., aphids, leafhoppers, planthoppers, beetles, and other insects.

Maize Dwarf Mosaic Virus (MDMV) is a long, flexuous, rod-shaped virus which is transmitted on seed. Early infection produces plants which are stunted and may never produce flowers and seed. MDMV symptoms are highly variable and include narrow, light green or yellowish streaks along the leaf veins and/or dark green “islands” on a yellow background. Symptoms may be present on all leaves that develop following infection. Both environmental conditions and the developmental stage at which the plant is infected may cause a reduction in yield; infection of young plants early in the season causes the most significant losses. In addition, early infection may predispose corn to root and stalk rots and premature death.

Environmental conditions also affect growth, development, and yield by altering pathogen activity or host physiology. The severity of the excess, deficiency, or imbalance of soil nutrients or water; extreme soil acidity or alkalinity; very high or low temperatures; air pollutants; or mechanical, pesticide, or other injury can be deadly at the seedling stage (Shurtleff, M. C. et al. (1980) Compendium of Corn Diseases, APS Press, St. Paul Minn., 105 pp.).

Corn Disease Control

To control corn diseases, it is necessary to disrupt disease development. Intervention requires understanding the pathogens, the spread of pathogens, disease cycles, and plant resistance. Disease control may be achieved by a single procedure (chemical sprays) or by the integrated use of environmental, genetic, and chemical factors. Successful, long-term disease control generally includes planting disease-resistant varieties, applying chemical control, and implementing sanitary cultural methods.

### SUMMARY OF THE INVENTION

The present invention provides nucleic acid sequences comprising corn seedling-derived polynucleotides (cdps) as presented in the Sequence Listing. Some of the cdps uniquely identify structural, functional, and regulatory genes of the corn seedling. The invention encompasses oligonucleotides, fragments, and derivatives of the cdps and provides nucleic acid sequences complementary to the nucleic acid sequences listed in the Sequence Listing.

The present invention further provides the following cdps of particular interest as identified by SEQ ID NO: 3537 (700162463H1), SEQ ID NO: 1161 (700158211H1), SEQ ID NO: 949 (700157856H1), SEQ ID NO: 3199 (700161822H1), SEQ ID NO: 1744 (700159434H1), SEQ ID NO: 1183 (700158258H1), SEQ ID NO: 377 (700156836H1), SEQ ID NO: 2501 (700160693H1), SEQ ID NO: 2527 (700160740H1), SEQ ID NO: 698 (700157394H1), SEQ ID NO: 2335 (700160435H1), SEQ ID NO: 1988 (700159867H1), SEQ ID NO: 2583 (700160844H1), SEQ ID NO: 585 (700157190H1), SEQ ID NO: 237 (700156608H1), SEQ ID NO: 2719 (700161045H1), SEQ ID NO: 266 (700156661H1), SEQ ID NO: 3021 (700161544H1), SEQ ID NO: 2040 (700159955H1), SEQ ID NO: 2224 (700160253H1), SEQ ID NO: 2761 (700161134H1), SEQ ID NO: 1166 (700158229H1), SEQ ID NO: 1164 (700158222H1), SEQ ID NO: 2479 (700160669H1), SEQ ID NO: 215 (700156563H1), SEQ ID NO: 2448 (700160623H1), SEQ ID NO: 2207 (700160227H1), SEQ ID NO: 2626 (700160906H1), SEQ ID NO: 1339 (700158532H1), SEQ ID NO: 2726 (700161054H1), SEQ ID NO: 336 (700156768H1), SEQ ID NO: 1963 (700159826H1), SEQ ID NO: 2285 (700160357H1), SEQ ID NO: 1969 (700159839H1), SEQ ID NO: 471 (700156989H1), SEQ ID NO: 3118 (700161689H1), SEQ ID NO: 2475 (700160665H1), SEQ ID NO: 2731 (700161064H1), SEQ ID NO: 1695 (700159330H1), SEQ ID NO: 35 (700142454H1), SEQ ID NO: 79 (700142522H1), SEQ ID NO: 2086 (700160030H1), SEQ ID NO: 2000 (700159882H1), SEQ ID NO: 1001 (700157954H1), SEQ ID NO: 1302 (700158460H1), SEQ ID NO: 1205 (700158305H1), SEQ ID NO: 2849 (700161279H1), SEQ ID NO: 2138 (700160112H1), SEQ ID NO: 224 (700156580H1), SEQ ID NO: 385 (700156847H1), SEQ ID NO: 3541 (700162468H1), SEQ ID NO: 3526 (700162448H1), SEQ ID NO: 2137 (700160111H1), SEQ ID NO: 1197 (700158287H1), SEQ ID NO: 2109 (700160067H1), SEQ ID NO: 1902 (700159726H1), SEQ ID NO: 2578 (700160832H1), SEQ ID NO: 1044 (700158022H1), SEQ ID NO: 956 (700157865H1), SEQ ID NO: 149 (700156439H1), SEQ ID NO: 1548 (700159028H1), SEQ ID NO: 3030 (700161557H1), SEQ ID NO: 2128 (700160091H1), SEQ ID NO: 3025 (700161550H1), SEQ ID NO: 195 (700156524H1), SEQ ID NO: 1385 (700158604H1), SEQ ID NO: 1091 (700158090H1), SEQ ID NO: 1517 (700158965H1), SEQ ID NO: 1439 (700158806H1), SEQ ID NO: 2755 (700161125H1), SEQ ID NO: 1449 (700158823H1), SEQ ID NO: 107 (700142566H1), SEQ ID NO: 2916 (700161388H1), SEQ ID NO: 3160 (700161766H1), SEQ ID NO: 3001 (700161515H1), SEQ ID NO: 1940 (700159780H1), SEQ ID NO: 2918 (700161390H1), SEQ ID NO: 426 (700156915H1), SEQ ID NO: 1024 (700157988H1), SEQ ID NO: 2468 (700160654H1), SEQ ID NO: 1886 (700159694H1), SEQ ID NO: 1160 (700158208H1), SEQ ID NO: 411 (700156889H1), SEQ ID NO: 1570 (700159068H1), SEQ ID NO: 3371 (700162137H1), SEQ ID NO: 1942 (700159783H1), SEQ ID NO: 3132 (700161719H1), SEQ ID NO: 138 (700156425H1), SEQ ID NO: 2870 (700161314H1), SEQ ID NO: 2624 (700160904H1), SEQ ID NO: 3518 (700162435H1), SEQ ID NO: 2931 (700161413H1), SEQ ID NO: 1649 (700159219H1), SEQ ID NO: 2477 (700160667H1), SEQ ID NO: 1513 (700158949H1), SEQ ID NO: 3340 (700162062H1), SEQ ID NO: 884 (700157746H1), SEQ ID NO: 1701 (700159340H1), SEQ ID NO: 2480 (700160671H1), SEQ ID NO: 938 (700157837H1), SEQ ID NO: 2834 (700161257H1), SEQ ID NO: 1020 (700157981H1), SEQ ID NO: 2272 (700160340H1), SEQ ID NO: 2055 (700159981H1), SEQ ID NO: 489 (700157022H1), SEQ ID NO: 1294 (700158449H1), SEQ ID NO: 1597 (700159125H1), SEQ ID NO: 1172 (700158240H1), SEQ ID NO: 1243 (700158366H1), SEQ ID NO: 2482 (700160674H1), SEQ ID NO: 2544 (700160768H1), SEQ ID NO: 2862 (700161296H1), SEQ ID NO: 2704 (700161024H1), SEQ ID NO: 2369 (700160492H1), SEQ ID NO: 3242 (700161894H1), SEQ ID NO: 2948 (700161439H1), SEQ ID NO: 1315 (700158484H1), SEQ ID NO: 2700 (700161016H1), SEQ ID NO: 1436 (700158695H1), SEQ ID NO: 1996 (700159877H1), SEQ ID NO: 172 (700156474H1), SEQ ID NO: 1787 (700159515H1), SEQ ID NO: 1929 (700159766H1), SEQ ID NO: 3601 (700162584H1), SEQ ID NO: 1615 (700159167H1), SEQ ID NO: 852 (700157686H1), SEQ ID NO: 2267 (700160335H1), SEQ ID NO: 2887 (700161344H1), SEQ ID NO: 803 (700157615H1), SEQ ID NO: 1878 (700159679H1), SEQ ID NO: 2026 (700159930H1), SEQ ID NO: 1250 (700158376H1), SEQ ID NO: 573 (700157172H1), SEQ ID NO: 105 (700142562H1), SEQ ID NO: 2205 (700160225H1), SEQ ID NO: 2389 (700160522H1), SEQ ID NO: 2876 (700161326H1), SEQ ID NO: 62 (700142492H1), SEQ ID NO: 2469 (700160658H1), SEQ ID NO: 1206 (700158310H1), SEQ ID NO: 666 (700157334H1), SEQ ID NO: 179 (700156486H1), SEQ ID NO: 1378 (700158590H1), SEQ ID NO: 3316 (700162025H1), SEQ ID NO: 578 (700157181H1), SEQ ID NO: 2433 (700160601H1), SEQ ID NO: 865 (700157712H1), SEQ ID NO: 1726 (700159386H1), SEQ ID NO: 3165 (700161772H1), SEQ ID NO: 1409 (700158643H1), SEQ ID NO: 2183 (700160186H1), SEQ ID NO: 2977 (700161476H1), SEQ ID NO: 1985 (700159863H1), SEQ ID NO: 768 (700157547H1), SEQ ID NO: 1499 (700158924H1), SEQ ID NO: 1404 (700158632H1), SEQ ID NO: 374 (700156831H1), SEQ ID NO: 1908 (700159733H1), SEQ ID NO: 1405 (700158636H1), SEQ ID NO: 1543 (700159020H1), SEQ ID NO: 2139 (700160115H1), SEQ ID NO: 1258 (700158387H1), SEQ ID NO: 3344 (700162066H1), SEQ ID NO: 3580 (700162539H1), SEQ ID NO: 1830 (700159602H1), SEQ ID NO: 3531 (700162455H1), SEQ ID NO: 515 (700157065H1), SEQ ID NO: 2723 (700161051H1), SEQ ID NO: 191 (700156519H1), SEQ ID NO: 3259 (700161925H1), SEQ ID NO: 622 (700157251H1), SEQ ID NO: 68 (700142503H1), SEQ ID NO: 32 (700142451H1), SEQ ID NO: 80 (700142523H1), SEQ ID NO: 2225 (700160259H1), SEQ ID NO: 37 (700142458H1), SEQ ID NO: 559 (700157147H1), SEQ ID NO: 581 (700157185H1), SEQ ID NO: 3042 (700161571H1), SEQ ID NO: 2789 (700161181H1), SEQ ID NO: 2692 (700161003H1), SEQ ID NO: 1493 (700158915H1), SEQ ID NO: 2431 (700160595H1), SEQ ID NO: 270 (700156667H1), SEQ ID NO: 2010 (700159905H1), SEQ ID NO: 3401 (700162185H1), SEQ ID NO: 3501 (700162392H1), SEQ ID NO: 307 (700156727H1), SEQ ID NO: 2830 (700161249H1), SEQ ID NO: 3464 (700162321H1), SEQ ID NO: 1034 (700158010H1), SEQ ID NO: 2573 (700160826H1), SEQ ID NO: 1657 (700159236H1), SEQ ID NO: 1646 (700159214H1), SEQ ID NO: 198 (700156531H1), SEQ ID NO: 151 (700156441H1), SEQ ID NO: 3257 (700161923H1), SEQ ID NO: 3553 (700162485H1), SEQ ID NO: 1839 (700159614H1), SEQ ID NO: 3056 (700161591H1), SEQ ID NO: 3320 (700162032H1), SEQ ID NO: 629 (700157262H1), SEQ ID NO: 1889 (700159703H1), SEQ ID NO: 2628 (700160908H1), SEQ ID NO: 1670 (700159272H1), SEQ ID NO: 1274 (700158421H1), SEQ ID NO: 1039 (700158016H1), SEQ ID NO: 1975 (700159850H1), SEQ ID NO: 2951 (700161442H1), SEQ ID NO: 1042 (700158020H1), SEQ ID NO: 1521 (700158980H1), SEQ ID NO: 1119 (700158143H1), SEQ ID NO: 277 (700156677H1), SEQ ID NO: 519 (700157072H1), SEQ ID NO: 1422 (700158672H1), SEQ ID NO: 2315 (700160405H1), SEQ ID NO: 922 (700157810H1), SEQ ID NO: 1175 (700158245H1), SEQ ID NO: 1763 (700159461H1), SEQ ID NO: 701 (700157405H1), SEQ ID NO: 2540 (700160761H1), SEQ ID NO: 1157 (700158204H1), SEQ ID NO: 576 (700157177H1), SEQ ID NO: 356 (700156801H1), SEQ ID NO: 1375 (700158585H1), SEQ ID NO: 3146 (700161738H1), SEQ ID NO: 3150 (700161744H1), SEQ ID NO: 1464 (700158854H1), SEQ ID NO: 2963 (700161457H1), SEQ ID NO: 2213 (700160236H1), SEQ ID NO: 1128 (700158158H1), SEQ ID NO: 3528 (700162450H1), SEQ ID NO: 1730 (700159395H1), SEQ ID NO: 3168 (700161775H1), SEQ ID NO: 918 (700157804H1), SEQ ID NO: 1775 (700159482H1), SEQ ID NO: 2676 (700160978H1), SEQ ID NO: 1567 (700159064H1), SEQ ID NO: 2412 (700160563H1), SEQ ID NO: 1003 (700157957H1), SEQ ID NO: 109 (700142571H1), SEQ ID NO: 1589 (700159110H1), SEQ ID NO: 508 (700157053H1), SEQ ID NO: 354 (700156792H1), SEQ ID NO: 505 (700157045H1), SEQ ID NO: 1307 (700158470H1), SEQ ID NO: 600 (700157219H1), SEQ ID NO: 1121 (700158145H1), SEQ ID NO: 1377 (700158588H1), SEQ ID NO: 2710 (700161034H1), SEQ ID NO: 1843 (700159620H1), SEQ ID NO: 998 (700157949H1), SEQ ID NO: 3564 (700162513H1), SEQ ID NO: 2708 (700161029H1), SEQ ID NO: 796 (700157593H1), SEQ ID NO: 1346 (700158541H1), SEQ ID NO: 2594 (700160860H1), SEQ ID NO: 898 (700157768H1), SEQ ID NO: 3384 (700162163H1), SEQ ID NO: 1552 (700159036H1), SEQ ID NO: 1554 (700159038H1), SEQ ID NO: 1712 (700159356H1), SEQ ID NO: 3094 (700161653H1), SEQ ID NO: 3525 (700162447H1), SEQ ID NO: 1858 (700159650H1), SEQ ID NO: 3428 (700162243H1), SEQ ID NO: 1997 (700159879H1), SEQ ID NO: 2451 (700160628H1), SEQ ID NO: 429 (700156922H1), SEQ ID NO: 1906 (700159731H1), SEQ ID NO: 2660 (700160954H1), SEQ ID NO: 1557 (700159046H1), SEQ ID NO: 2672 (700160972H1), SEQ ID NO: 1066 (700158055H1), SEQ ID NO: 3449 (700162285H1), SEQ ID NO: 1693 (700159327H1), SEQ ID NO: 601 (700157222H1), SEQ ID NO: 3527 (700162449H1), SEQ ID NO: 85 (700142532H1), SEQ ID NO: 2062 (700159989H1), SEQ ID NO: 1862 (700159656H1), SEQ ID NO: 3544 (700162472H1), SEQ ID NO: 279 (700156681H1), SEQ ID NO: 2842 (700161267H1), SEQ ID NO: 1210 (700158315H1), SEQ ID NO: 2456 (700160640H1), SEQ ID NO: 2620 (700160893H1), SEQ ID NO: 724 (700157446H1), SEQ ID NO: 615 (700157240H1), SEQ ID NO: 1694 (700159328H1), SEQ ID NO: 500 (700157039H1), SEQ ID NO: 2781 (700161170H1), SEQ ID NO: 2909 (700161381H1), SEQ ID NO: 436 (700156929H1), SEQ ID NO: 1036 (700158013H1), SEQ ID NO: 1736 (700159416H1), SEQ ID NO: 2569 (700160819H1), SEQ ID NO: 3313 (700162020H1), SEQ ID NO: 1073 (700158065H1), SEQ ID NO: 1990 (700159869H1), SEQ ID NO: 88 (700142539H1), SEQ ID NO: 1704 (700159347H1), SEQ ID NO: 1158 (700158205H1), SEQ ID NO: 3586 (700162548H1), SEQ ID NO: 1901 (700159724H1), SEQ ID NO: 1994 (700159875H1), SEQ ID NO: 3142 (700161734H1), SEQ ID NO: 2907 (700161378H1), SEQ ID NO: 1937 (700159776H1), SEQ ID NO: 2767 (700161143H1), SEQ ID NO: 2032 (700159941H1), SEQ ID NO: 3099 (700161661H1), SEQ ID NO: 2828 (700161245H1), SEQ ID NO: 437 (700156933H1), SEQ ID NO: 2985 (700161490H1), SEQ ID NO: 2499 (700160691H1), SEQ ID NO: 2336 (700160436H1), SEQ ID NO: 1074 (700158066H1), SEQ ID NO: 2073 (700160007H1), SEQ ID NO: 2956 (700161448H1), SEQ ID NO: 1089 (700158086H1), SEQ ID NO: 2195 (700160211H1), SEQ ID NO: 1741 (700159425H1), SEQ ID NO: 607 (700157229H1), SEQ ID NO: 2840 (700161265H1), SEQ ID NO: 2649 (700160942H1), SEQ ID NO: 1221 (700158332H1), SEQ ID NO: 1813 (700159568H1), SEQ ID NO: 1805 (700159552H1), SEQ ID NO: 285 (700156691H1), SEQ ID NO: 294 (700156709H1), SEQ ID NO: 3291 (700161979H1), SEQ ID NO: 2634 (700160921H1), SEQ ID NO: 3039 (700161567H1), SEQ ID NO: 3054 (700161589H1), SEQ ID NO: 453 (700156964H1), SEQ ID NO: 2681 (700160985H1), SEQ ID NO: 937 (700157836H1), SEQ ID NO: 1703 (700159346H1), SEQ ID NO: 2829 (700161247H1), SEQ ID NO: 3120 (700161693H1), SEQ ID NO: 552 (700157137H1), SEQ ID NO: 1431 (700158688H1), SEQ ID NO: 1806 (700159554H1), SEQ ID NO: 1814 (700159570H1), SEQ ID NO: 3308 (700162013H1), SEQ ID NO: 167 (700156463H1), SEQ ID NO: 2059 (700159986H1), SEQ ID NO: 242 (700156618H1), SEQ ID NO: 1256 (700158383H1), SEQ ID NO: 744 (700157489H1), SEQ ID NO: 1895 (700159716H1), SEQ ID NO: 440 (700156938H1), SEQ ID NO: 3217 (700161849H1), SEQ ID NO: 2831 (700161251H1), SEQ ID NO: 1015 (700157975H1), SEQ ID NO: 1366 (700158568H1), SEQ ID NO: 314 (700156740H1), SEQ ID NO: 2188 (700160195H1), SEQ ID NO: 84 (700142528H1), SEQ ID NO: 3069 (700161614H1), SEQ ID NO: 2952 (700161443H1), SEQ ID NO: 53 (700142478H1), SEQ ID NO: 1778 (700159492H1), SEQ ID NO: 1881 (700159685H1), SEQ ID NO: 1907 (700159732H1), SEQ ID NO: 614 (700157239H1), SEQ ID NO: 3395 (700162178H1), SEQ ID NO: 1312 (700158481H1), SEQ ID NO: 243 (700156621H1), SEQ ID NO: 1131 (700158163H1), SEQ ID NO: 678 (700157359H1), SEQ ID NO: 1506 (700158934H1), SEQ ID NO: 2643 (700160932H1), SEQ ID NO: 2593 (700160857H1), SEQ ID NO: 142 (700156430H1), SEQ ID NO: 2371 (700160494H1), SEQ ID NO: 925 (700157817H1), SEQ ID NO: 3041 (700161570H1), SEQ ID NO: 3050 (700161583H1), SEQ ID NO: 3079 (700161637H1), SEQ ID NO: 1245 (700158369H1), SEQ ID NO: 2309 (700160391H1), SEQ ID NO: 2713 (700161037H1), SEQ ID NO: 1289 (700158440H1), SEQ ID NO: 635 (700157268H1), SEQ ID NO: 653 (700157309H1), SEQ ID NO: 244 (700156623H1), SEQ ID NO: 338 (700156771H1), SEQ ID NO: 1406 (700158637H1), SEQ ID NO: 2941 (700161430H1), SEQ ID NO: 2516 (700160725H1), SEQ ID NO: 1945 (700159791H1), SEQ ID NO: 1485 (700158902H1), SEQ ID NO: 3045 (700161576H1), SEQ ID NO: 1271 (700158417H1), SEQ ID NO: 2786 (700161176H1), SEQ ID NO: 156 (700156447H1), SEQ ID NO: 1367 (700158574H1), SEQ ID NO: 3169 (700161776H1), SEQ ID NO: 2076 (700160012H1), SEQ ID NO: 3253 (700161917H1), SEQ ID NO: 950 (700157857H1), SEQ ID NO: 1237 (700158356H1), SEQ ID NO: 2063 (700159990H1), SEQ ID NO: 3574 (700162527H1), SEQ ID NO: 580 (700157183H1), SEQ ID NO: 2034 (700159943H1), SEQ ID NO: 892 (700157757H1), SEQ ID NO: 1096 (700158104H1), SEQ ID NO: 1883 (700159691H1), SEQ ID NO: 2966 (700161460H1), SEQ ID NO: 1632 (700159193H1), SEQ ID NO: 1811 (700159565H1), SEQ ID NO: 1803 (700159549H1), SEQ ID NO: 2390 (700160523H1), SEQ ID NO: 2329 (700160426H1), SEQ ID NO: 2835 (700161259H1), SEQ ID NO: 1428 (700158681H1), SEQ ID NO: 2016 (700159915H1), SEQ ID NO: 3487 (700162367H1), SEQ ID NO: 2699 (700161014H1), SEQ ID NO: 2694 (700161006H1), SEQ ID NO: 163 (700156456H1), SEQ ID NO: 2302 (700160381H1), SEQ ID NO: 23 (700142438H1), SEQ ID NO: 1720 (700159376H1), SEQ ID NO: 1519 (700158975H1), SEQ ID NO: 569 (700157166H1), SEQ ID NO: 3015 (700161536H1), SEQ ID NO: 521 (700157076H1), SEQ ID NO: 736 (700157474H1), SEQ ID NO: 626 (700157256H1), SEQ ID NO: 1846 (700159627H1), SEQ ID NO: 1642 (700159209H1), SEQ ID NO: 2391 (700160526H1), SEQ ID NO: 2410 (700160559H1), SEQ ID NO: 2061 (700159988H1), SEQ ID NO: 413 (700156891H1), SEQ ID NO: 1072 (700158064H1), SEQ ID NO: 1038 (700158015H1), SEQ ID NO: 1854 (700159641H1), SEQ ID NO: 143 (700156431H1), SEQ ID NO: 81 (700142524H1), SEQ ID NO: 3111 (700161679H1), SEQ ID NO: 2806 (700161213H1), SEQ ID NO: 139 (700156426H1), SEQ ID NO: 43 (700142466H1), SEQ ID NO: 185 (700156495H1), SEQ ID NO: 2524 (700160737H1), SEQ ID NO: 64 (700142494H1), SEQ ID NO: 3171 (700161778H1), SEQ ID NO: 2707 (700161028H1), SEQ ID NO: 2033 (700159942H1), SEQ ID NO: 639 (700157276H1), SEQ ID NO: 532 (700157093H1), SEQ ID NO: 3155 (700161756H1), SEQ ID NO: 1338 (700158531H1), SEQ ID NO: 575 (700157176H1), SEQ ID NO: 1177 (700158248H1), SEQ ID NO: 1099 (700158107H1), SEQ ID NO: 1861 (700159654H1), SEQ ID NO: 1832 (700159606H1), SEQ ID NO: 829 (700157650H1), SEQ ID NO: 878 (700157737H1), SEQ ID NO: 1443 (700158812H1), SEQ ID NO: 329 (700156761H1), SEQ ID NO: 3227 (700161866H1), SEQ ID NO: 851 (700157685H1), SEQ ID NO: 2728 (700161056H1), SEQ ID NO: 2854 (700161284H1), SEQ ID NO: 3547 (700162477H1), SEQ ID NO: 2393 (700160529H1), SEQ ID NO: 323 (700156754H1), SEQ ID NO: 1407 (700158638H1), SEQ ID NO: 187 (700156506H1), SEQ ID NO: 441 (700156940H1), SEQ ID NO: 1904 (700159728H1), SEQ ID NO: 1386 (700158605H1), SEQ ID NO: 3323 (700162037H1), SEQ ID NO: 2576 (700160830H1), SEQ ID NO: 1283 (700158434H1), SEQ ID NO: 2048 (700159970H1), SEQ ID NO: 2123 (700160085H1), SEQ ID NO: 2305 (700160385H1), SEQ ID NO: 1191 (700158278H1), SEQ ID NO: 1420 (700158669H1), SEQ ID NO: 335 (700156767H1), SEQ ID NO: 2889 (700161346H1), SEQ ID NO: 798 (700157601H1), SEQ ID NO: 976 (700157911H1), SEQ ID NO: 104 (700142561H1), SEQ ID NO: 1052 (700158034H1), SEQ ID NO: 1347 (700158542H1), SEQ ID NO: 1653 (700159226H1), SEQ ID NO: 958 (700157868H1), SEQ ID NO: 2252 (700160313H1), SEQ ID NO: 2181 (700160184H1), SEQ ID NO: 971 (700157895H1), SEQ ID NO: 2492 (700160684H1), SEQ ID NO: 1043 (700158021H1), SEQ ID NO: 12 (700142422H1), SEQ ID NO: 592 (700157207H1), SEQ ID NO: 3163 (700161770H1), SEQ ID NO: 3331 (700162049H1), SEQ ID NO: 2591 (700160855H1), SEQ ID NO: 2153 (700160140H1), SEQ ID NO: 1917 (700159748H1), SEQ ID NO: 2824 (700161241H1), SEQ ID NO: 3407 (700162206H1), SEQ ID NO: 1453 (700158832H1), SEQ ID NO: 2747 (700161102H1), SEQ ID NO: 2801 (700161206H1), SEQ ID NO: 1558 (700159047H1), SEQ ID NO: 2888 (700161345H1), SEQ ID NO: 1681 (700159307H1), SEQ ID NO: 2330 (700160427H1), SEQ ID NO: 1607 (700159151H1), SEQ ID NO: 1635 (700159196H1), SEQ ID NO: 2115 (700160076H1), SEQ ID NO: 656 (700157319H1), SEQ ID NO: 888 (700157751H1), SEQ ID NO: 711 (700157424H1), SEQ ID NO: 2307 (700160387H1), SEQ ID NO: 145 (700156433H1), SEQ ID NO: 1734 (700159411H1), SEQ ID NO: 342 (700156776H1), SEQ ID NO: 1842 (700159619H1), SEQ ID NO: 1360 (700158558H1), SEQ ID NO: 799 (700157605H1), SEQ ID NO: 1759 (700159454H1), SEQ ID NO: 2486 (700160678H1), SEQ ID NO: 3194 (700161816H1), SEQ ID NO: 1457 (700158839H1), SEQ ID NO: 2666 (700160964H1), SEQ ID NO: 3509 (700162417H1), SEQ ID NO: 2430 (700160593H1), SEQ ID NO: 446 (700156951H1), SEQ ID NO: 3273 (700161948H1), SEQ ID NO: 822 (700157642H1), SEQ ID NO: 1981 (700159857H1), SEQ ID NO: 1146 (700158184H1), SEQ ID NO: 3594 (700162570H1), SEQ ID NO: 2179 (700160180H1), SEQ ID NO: 1195 (700158285H1), SEQ ID NO: 3489 (700162376H1), SEQ ID NO: 2192 (700160204H1), SEQ ID NO: 3023 (700161547H1), SEQ ID NO: 363 (700156813H1), SEQ ID NO: 2498 (700160690H1), SEQ ID NO: 2491 (700160683H1), SEQ ID NO: 1875 (700159673H1), SEQ ID NO: 127 (700156407H1), SEQ ID NO: 1381 (700158596H1), SEQ ID NO: 1630 (700159187H1), SEQ ID NO: 3325 (700162042H1), SEQ ID NO: 3444 (700162277H1), SEQ ID NO: 1705 (700159348H1), SEQ ID NO: 2533 (700160750H1), SEQ ID NO: 1986 (700159864H1), SEQ ID NO: 2348 (700160459H1), SEQ ID NO: 3019 (700161541H1), SEQ ID NO: 2557 (700160795H1), SEQ ID NO: 3212 (700161842H1), SEQ ID NO: 3202 (700161826H1), SEQ ID NO: 1745 (700159435H1), SEQ ID NO: 188 (700156511H1), SEQ ID NO: 964 (700157879H1), SEQ ID NO: 3470 (700162338H1), SEQ ID NO: 3603 (700162591H1), SEQ ID NO: 2500 (700160692H1), SEQ ID NO: 2775 (700161157H1), SEQ ID NO: 2778 (700161166H1), SEQ ID NO: 1740 (700159423H1), SEQ ID NO: 1576 (700159081H1), SEQ ID NO: 3508 (700162414H1), SEQ ID NO: 3484 (700162364H1), SEQ ID NO: 1054 (700158037H1), SEQ ID NO: 4316 (700454828H1), SEQ ID NO: 4594 (700455401H1), SEQ ID NO: 4100 (700454421H1), SEQ ID NO: 5528 (700457192H1), SEQ ID NO: 5760 (700457654H1), SEQ ID NO: 3797 (700453825H1), SEQ ID NO: 5257 (700456691H1), SEQ ID NO: 3788 (700453810H1), SEQ ID NO: 4097 (700454418H1), SEQ ID NO: 5731 (700457589H1), SEQ ID NO: 5009 (700456178H1), SEQ ID NO: 4972 (700456102H1), SEQ ID NO: 5423 (700457018H1), SEQ ID NO: 4519 (700455175H1), SEQ ID NO: 4729 (700455675H1), SEQ ID NO: 5640 (700457388H1), SEQ ID NO: 6092 (700458353H1), SEQ ID NO: 4157 (700454513H1), SEQ ID NO: 6103 (700458378H1), SEQ ID NO: 3821 (700453864H1), SEQ ID NO: 4968 (700456094H1), SEQ ID NO: 4292 (700454771H1), SEQ ID NO: 5425 (700457023H1), SEQ ID NO: 5154 (700456454H1), SEQ ID NO: 5445 (700457055H1), SEQ ID NO: 5146 (700456444H1), SEQ ID NO: 4643 (700455525H1), SEQ ID NO: 5267 (700456715H1), SEQ ID NO: 4380 (700454949H1), SEQ ID NO: 3883 (700453981H1), SEQ ID NO: 5847 (700457846H1), SEQ ID NO: 4889 (700455957H1), SEQ ID NO: 5239 (700456657H1), SEQ ID NO: 5072 (700456315H1), SEQ ID NO: 6018 (700458195H1), SEQ ID NO: 5205 (700456575H1), SEQ ID NO: 5826 (700457804H1), SEQ ID NO: 4475 (700455102H1), SEQ ID NO: 5429 (700457030H1), SEQ ID NO: 5452 (700457062H1), SEQ ID NO: 5453 (700457063H1), SEQ ID NO: 4131 (700454465H1), SEQ ID NO: 5538 (700457213H1), SEQ ID NO: 5540 (700457215H1), SEQ ID NO: 5433 (700457035H1), SEQ ID NO: 3984 (700454206H1), SEQ ID NO: 5369 (700456891H1), SEQ ID NO: 5766 (700457667H1), SEQ ID NO: 5699 (700457519H1), SEQ ID NO: 5698 (700457518H1), SEQ ID NO: 5907 (700457953H1), SEQ ID NO: 5829 (700457808H1), SEQ ID NO: 5872 (700457887H1), SEQ ID NO: 4247 (700454678H1), SEQ ID NO: 5225 (700456633H1), SEQ ID NO: 4823 (700455851H1), SEQ ID NO: 4083 (700454394H1), SEQ ID NO: 4084 (700454395H1), SEQ ID NO: 4030 (700454295H1), SEQ ID NO: 5636 (700457380H1), SEQ ID NO: 4784 (700455780H1), SEQ ID NO: 5925 (700457993H1), SEQ ID NO: 6119 (700458416H1), SEQ ID NO: 6124 (700458424H1), SEQ ID NO: 4463 (700455081H1), SEQ ID NO: 5554 (700457243H1), SEQ ID NO: 5497 (700457136H1), SEQ ID NO: 5602 (700457326H1), SEQ ID NO: 5651 (700457411H1), SEQ ID NO: 4522 (700455178H1), SEQ ID NO: 5647 (700457403H1), SEQ ID NO: 5426 (700457025H1), SEQ ID NO: 5395 (700456958H1), SEQ ID NO: 5422 (700457017H1), SEQ ID NO: 6127 (700458428H1), SEQ ID NO: 6121 (700458420H1), SEQ ID NO: 6128 (700458429H1), SEQ ID NO: 4915 (700456004H1), SEQ ID NO: 5183 (700456520H1), SEQ ID NO: 5179 (700456505H1), SEQ ID NO: 5480 (700457107H1), SEQ ID NO: 6070 (700458311H1), SEQ ID NO: 4263 (700454707H1), SEQ ID NO: 4325 (700454841H1), SEQ ID NO: 5552 (700457241H1), SEQ ID NO: 6241 (700458630H1), SEQ ID NO: 4853 (700455908H1), SEQ ID NO: 5088 (700456339H1), SEQ ID NO: 5082 (700456331H1), SEQ ID NO: 4419 (700455012H1), SEQ ID NO: 5131 (700456416H1), SEQ ID NO: 4417 (700455010H1), SEQ ID NO: 4137 (700454471H1), SEQ ID NO: 4786 (700455782H1), SEQ ID NO: 5221 (700456626H1), SEQ ID NO: 4095 (700454414H1), SEQ ID NO: 4453 (700455062H1), SEQ ID NO: 5253 (700456683H1), SEQ ID NO: 4588 (700455290H1), SEQ ID NO: 4456 (700455068H1), SEQ ID NO: 4428 (700455024H1), SEQ ID NO: 3825 (700453874H1), SEQ ID NO: 4686 (700455610H1), SEQ ID NO: 4711 (700455650H1), SEQ ID NO: 4067 (700454365H1), SEQ ID NO: 4436 (700455038H1), SEQ ID NO: 6010 (700458175H1), SEQ ID NO: 4256 (700454692H1), SEQ ID NO: 5474 (700457092H1), SEQ ID NO: 4656 (700455554H1), SEQ ID NO: 4832 (700455867H1), SEQ ID NO: 5744 (700457627H1), SEQ ID NO: 5226 (700456634H1), SEQ ID NO: 4529 (700455189H1), SEQ ID NO: 5471 (700457085H1), SEQ ID NO: 5451 (700457061H1), SEQ ID NO: 5828 (700457807H1), SEQ ID NO: 5771 (700457674H1), SEQ ID NO: 4957 (700456074H1), SEQ ID NO: 4985 (700456127H1), SEQ ID NO: 4981 (700456114H1), SEQ ID NO: 3971 (700454173H1), SEQ ID NO: 4060 (700454351H1), SEQ ID NO: 4734 (700455681H1), SEQ ID NO: 5136 (700456423H1), SEQ ID NO: 5680 (700457471H1), SEQ ID NO: 5980 (700458123H1), SEQ ID NO: 3940 (700454119H1), SEQ ID NO: 3866 (700453958H1), SEQ ID NO: 5861 (700457867H1), SEQ ID NO: 3836 (700453895H1), SEQ ID NO: 5458 (700457072H1), SEQ ID NO: 4366 (700454923H1), SEQ ID NO: 4211 (700454614H1), SEQ ID NO: 6273 (700458684H1), SEQ ID NO: 5875 (700457891H1), SEQ ID NO: 5140 (700456433H1), SEQ ID NO: 5986 (700458136H1), SEQ ID NO: 6262 (700458668H1), SEQ ID NO: 5247 (700456675H1), SEQ ID NO: 5721 (700457565H1), SEQ ID NO: 4531 (700455195H1), SEQ ID NO: 5577 (700457273H1), SEQ ID NO: 4399 (700454984H1), SEQ ID NO: 6072 (700458313H1), SEQ ID NO: 4442 (700455048H1), SEQ ID NO: 4078 (700454381H1), SEQ ID NO: 4751 (700455713H1), SEQ ID NO: 5790 (700457717H1), SEQ ID NO: 5406 (700456977H1), SEQ ID NO: 4532 (700455196H1), SEQ ID NO: 4012 (700454262H1), SEQ ID NO: 5556 (700457245H1), SEQ ID NO: 5262 (700456707H1), SEQ ID NO: 4900 (700455974H1), SEQ ID NO: 5290 (700456752H1), SEQ ID NO: 3957 (700454145H1), SEQ ID NO: 6291 (700458826H1), SEQ ID NO: 6292 (700458827H1), SEQ ID NO: 4639 (700455517H1), SEQ ID NO: 6137 (700458443H1), SEQ ID NO: 4367 (700454925H1), SEQ ID NO: 4467 (700455085H1), SEQ ID NO: 5473 (700457090H1), SEQ ID NO: 5482 (700457115H1), SEQ ID NO: 4266 (700454717H1), SEQ ID NO: 4272 (700454725H1), SEQ ID NO: 4356 (700454909H1), SEQ ID NO: 4135 (700454469H1), SEQ ID NO: 5259 (700456694H1), SEQ ID NO: 5736 (700457604H1), SEQ ID NO: 5114 (700456381H1), SEQ ID NO: 5656 (700457419H1), SEQ ID NO: 4512 (700455167H1), SEQ ID NO: 4460 (700455077H1), SEQ ID NO: 4553 (700455237H1), SEQ ID NO: 4373 (700454937H1), SEQ ID NO: 4400 (700454985H1), SEQ ID NO: 5379 (700456917H1), SEQ ID NO: 5678 (700457466H1), SEQ ID NO: 5746 (700457629H1), SEQ ID NO: 5152 (700456452H1), SEQ ID NO: 5133 (700456418H1), SEQ ID NO: 4908 (700455987H1), SEQ ID NO: 5650 (700457409H1), SEQ ID NO: 6060 (700458287H1), SEQ ID NO: 6011 (700458178H1), SEQ ID NO: 4271 (700454724H1), SEQ ID NO: 5489 (700457124H1), SEQ ID NO: 5762 (700457658H1), SEQ ID NO: 4068 (700454366H1), SEQ ID NO: 4075 (700454377H1), SEQ ID NO: 3910 (700454049H1), SEQ ID NO: 4394 (700454970H1), SEQ ID NO: 5450 (700457060H1), SEQ ID NO: 5719 (700457561H1), SEQ ID NO: 5763 (700457660H1), SEQ ID NO: 4725 (700455669H1), SEQ ID NO: 4003 (700454241H1), SEQ ID NO: 5487 (700457122H1), SEQ ID NO: 5264 (700456711H1), SEQ ID NO: 5850 (700457850H1), SEQ ID NO: 5501 (700457141H1), SEQ ID NO: 4085 (700454396H1), SEQ ID NO: 4091 (700454409H1), SEQ ID NO: 4404 (700454989H1), SEQ ID NO: 5390 (700456948H1), SEQ ID NO: 5751 (700457638H1), SEQ ID NO: 4914 (700456003H1), SEQ ID NO: 6035 (700458234H1), SEQ ID NO: 6078 (700458322H1), SEQ ID NO: 5893 (700457927H1), SEQ ID NO: 5309 (700456784H1), SEQ ID NO: 4539 (700455212H1), SEQ ID NO: 5551 (700457240H1), SEQ ID NO: 5522 (700457174H1), SEQ ID NO: 5486 (700457121H1), SEQ ID NO: 4478 (700455106H1), SEQ ID NO: 4139 (700454474H1), SEQ ID NO: 5938 (700458026H1), SEQ ID NO: 6329 (700458889H1), SEQ ID NO: 5155 (700456455H1), SEQ ID NO: 5883 (700457911H1), SEQ ID NO: 3873 (700453966H1), SEQ ID NO: 4652 (700455543H1), SEQ ID NO: 4425 (700455021H1), SEQ ID NO: 4406 (700454992H1), SEQ ID NO: 6084 (700458339H1), SEQ ID NO: 3644 (700405359H1), SEQ ID NO: 5258 (700456692H1), SEQ ID NO: 4088 (700454405H1), SEQ ID NO: 3976 (700454186H1), SEQ ID NO: 4054 (700454337H1), SEQ ID NO: 4704 (700455637H1), SEQ ID NO: 3707 (700405462H1), SEQ ID NO: 6165 (700458510H1), SEQ ID NO: 6058 (700458285H1), SEQ ID NO: 4275 (700454729H1), SEQ ID NO: 3755 (700453757H1), SEQ ID NO: 5566 (700457256H1), SEQ ID NO: 4545 (700455226H1), SEQ ID NO: 4144 (700454483H1), SEQ ID NO: 5216 (700456614H1), SEQ ID NO: 4490 (700455127H1), SEQ ID NO: 5278 (700456729H1), SEQ ID NO: 5260 (700456695H1), SEQ ID NO: 5157 (700456458H1), SEQ ID NO: 5483 (700457116H1), SEQ ID NO: 4032 (700454303H1), SEQ ID NO: 5709 (700457540H1), SEQ ID NO: 4125 (700454456H1), SEQ ID NO: 4503 (700455152H1), SEQ ID NO: 4567 (700455262H1), SEQ ID NO: 5442 (700457051H1), SEQ ID NO: 5573 (700457266H1), SEQ ID NO: 5531 (700457202H1), SEQ ID NO: 4031 (700454302H1), SEQ ID NO: 5381 (700456920H1), SEQ ID NO: 6257 (700458655H1), SEQ ID NO: 4322 (700454836H1), SEQ ID NO: 6059 (700458286H1), SEQ ID NO: 5090 (700456344H1), SEQ ID NO: 4920 (700456014H1), SEQ ID NO: 4555 (700455240H1), SEQ ID NO: 6046 (700458257H1), SEQ ID NO: 3985 (700454209H1), SEQ ID NO: 3988 (700454213H1), SEQ ID NO: 5163 (700456469H1), SEQ ID NO: 5791 (700457720H1), SEQ ID NO: 4363 (700454917H1), SEQ ID NO: 4474 (700455101H1), SEQ ID NO: 4337 (700454859H1), SEQ ID NO: 4340 (700454864H1), SEQ ID NO: 3786 (700453808H1), SEQ ID NO: 3686 (700405427H1), SEQ ID NO: 5691 (700457504H1), SEQ ID NO: 4059 (700454350H1), SEQ ID NO: 4038 (700454313H1), SEQ ID NO: 4039 (700454314H1), SEQ ID NO: 5147 (700456445H1), SEQ ID NO: 4376 (700454942H1), SEQ ID NO: 3997 (700454229H1), SEQ ID NO: 3892 (700454004H1), SEQ ID NO: 5747 (700457630H1), SEQ ID NO: 4835 (700455873H1), SEQ ID NO: 3941 (700454121H1), SEQ ID NO: 3990 (700454221H1), SEQ ID NO: 5011 (700456186H1), SEQ ID NO: 5411 (700456986H1), SEQ ID NO: 5904 (700457948H1), SEQ ID NO: 5642 (700457392H1), SEQ ID NO: 4098 (700454419H1), SEQ ID NO: 5803 (700457750H1), SEQ ID NO: 4955 (700456069H1), SEQ ID NO: 5251 (700456679H1), SEQ ID NO: 6017 (700458194H1), SEQ ID NO: 4159 (700454516H1), SEQ ID NO: 4514 (700455169H1), SEQ ID NO: 5271 (700456720H1), SEQ ID NO: 4426 (700455022H1), SEQ ID NO: 4409 (700454996H1), SEQ ID NO: 4559 (700455247H1), SEQ ID NO: 5472 (700457088H1), SEQ ID NO: 3638 (700405352H1), SEQ ID NO: 5283 (700456741H1), SEQ ID NO: 5781 (700457692H1), SEQ ID NO: 5628 (700457367H1), SEQ ID NO: 5076 (700456324H1), SEQ ID NO: 5890 (700457924H1), SEQ ID NO: 4299 (700454785H1), SEQ ID NO: 3740 (700453730H1), SEQ ID NO: 5761 (700457656H1), SEQ ID NO: 5765 (700457663H1), SEQ ID NO: 4699 (700455630H1), SEQ ID NO: 4346 (700454880H1), SEQ ID NO: 6038 (700458240H1), SEQ ID NO: 4564 (700455257H1), SEQ ID NO: 5895 (700457929H1), SEQ ID NO: 5646 (700457402H1), SEQ ID NO: 4666 (700455575H1), SEQ ID NO: 4665 (700455574H1), SEQ ID NO: 3931 (700454106H1), SEQ ID NO: 4089 (700454406H1), SEQ ID NO: 3953 (700454139H1), SEQ ID NO: 3932 (700454107H1), SEQ ID NO: 3901 (700454033H1), SEQ ID NO: 3890 (700453995H1), SEQ ID NO: 5558 (700457247H1), SEQ ID NO: 4863 (700455922H1), SEQ ID NO: 3841 (700453912H1), SEQ ID NO: 6215 (700458576H1), SEQ ID NO: 4495 (700455139H1), SEQ ID NO: 5560 (700457250H1), SEQ ID NO: 3989 (700454219H1), SEQ ID NO: 6114 (700458404H1), SEQ ID NO: 5189 (700456528H1), SEQ ID NO: 5722 (700457568H1), SEQ ID NO: 4563 (700455255H1), SEQ ID NO: 6040 (700458242H1), SEQ ID NO: 5547 (700457230H1), SEQ ID NO: 4104 (700454426H1), SEQ ID NO: 4681 (700455604H1), SEQ ID NO: 6322 (700458877H1), SEQ ID NO: 3608 (700405305H1), SEQ ID NO: 4261 (700454705H1), SEQ ID NO: 5308 (700456783H1), SEQ ID NO: 5178 (700456503H1), SEQ ID NO: 3758 (700453762H1), SEQ ID NO: 3756 (700453759H1), SEQ ID NO: 3708 (700405463H1), SEQ ID NO: 5243 (700456663H1), SEQ ID NO: 6202 (700458557H1), SEQ ID NO: 6196 (700458549H1), SEQ ID NO: 5339 (700456844H1), SEQ ID NO: 4568 (700455263H1), SEQ ID NO: 5608 (700457336H1), SEQ ID NO: 4939 (700456049H1), SEQ ID NO: 5546 (700457225H1), SEQ ID NO: 5416 (700457006H1), SEQ ID NO: 5911 (700457961H1), SEQ ID NO: 5007 (700456175H1), SEQ ID NO: 5564 (700457254H1), SEQ ID NO: 5565 (700457255H1), SEQ ID NO: 5848 (700457848H1), SEQ ID NO: 4670 (700455579H1), SEQ ID NO: 5388 (700456942H1), SEQ ID NO: 4820 (700455846H1), SEQ ID NO: 5806 (700457760H1), SEQ ID NO: 5873 (700457888H1), SEQ ID NO: 5955 (700458063H1), SEQ ID NO: 5161 (700456463H1), SEQ ID NO: 4117 (700454445H1), SEQ ID NO: 5475 (700457093H1), SEQ ID NO: 3835 (700453894H1), SEQ ID NO: 3881 (700453979H1), SEQ ID NO: 5289 (700456751H1), SEQ ID NO: 4687 (700455611H1), SEQ ID NO: 5778 (700457688H1), SEQ ID NO: 5663 (700457434H1), SEQ ID NO: 6140 (700458451H1), SEQ ID NO: 4441 (700455046H1), SEQ ID NO: 4824 (700455853H1), SEQ ID NO: 3827 (700453878H1), SEQ ID NO: 4130 (700454464H1), SEQ ID NO: 4110 (700454435H1), SEQ ID NO: 5793 (700457726H1), SEQ ID NO: 5506 (700457147H1), SEQ ID NO: 6164 (700458509H1), SEQ ID NO: 4048 (700454329H1), SEQ ID NO: 5424 (700457020H1), SEQ ID NO: 5420 (700457012H1), SEQ ID NO: 6151 (700458468H1), SEQ ID NO: 4947 (700456059H1), SEQ ID NO: 4352 (700454902H1), SEQ ID NO: 6111 (700458395H1), SEQ ID NO: 6220 (700458587H1), SEQ ID NO: 6234 (700458616H1), SEQ ID NO: 4560 (700455252H1), SEQ ID NO: 4392 (700454967H1), SEQ ID NO: 4881 (700455944H1), SEQ ID NO: 4929 (700456036H1), SEQ ID NO: 5568 (700457258H1), SEQ ID NO: 5570 (700457260H1), SEQ ID NO: 5545 (700457223H1), SEQ ID NO: 4523 (700455179H1), SEQ ID NO: 5750 (700457637H1), SEQ ID NO: 4873 (700455936H1), SEQ ID NO: 6186 (700458537H1), SEQ ID NO: 6198 (700458552H1), SEQ ID NO: 5414 (700456994H1), SEQ ID NO: 5799 (700457740H1), SEQ ID NO: 5669 (700457445H1), SEQ ID NO: 5754 (700457644H1), SEQ ID NO: 4640 (700455518H1), SEQ ID NO: 3785 (700453807H1), SEQ ID NO: 6245 (700458639H1), SEQ ID NO: 3844 (700453919H1), SEQ ID NO: 3682 (700405418H1), SEQ ID NO: 3627 (700405340H1), SEQ ID NO: 5005 (700456169H1), SEQ ID NO: 4948 (700456061H1), SEQ ID NO: 4355 (700454907H1), SEQ ID NO: 4894 (700455964H1), SEQ ID NO: 5485 (700457120H1), SEQ ID NO: 4586 (700455286H1), SEQ ID NO: 4410 (700455001H1), SEQ ID NO: 4375 (700454941H1), SEQ ID NO: 4251 (700454684H1), SEQ ID NO: 6278 (700458802H1), SEQ ID NO: 4880 (700455943H1), SEQ ID NO: 5092 (700456346H1), SEQ ID NO: 4830 (700455863H1), SEQ ID NO: 4827 (700455858H1), SEQ ID NO: 5219 (700456619H1), SEQ ID NO: 5629 (700457371H1), SEQ ID NO: 5404 (700456971H1), SEQ ID NO: 4525 (700455183H1), SEQ ID NO: 4312 (700454820H1), SEQ ID NO: 3860 (700453944H1), SEQ ID NO: 5676 (700457457H1), SEQ ID NO: 5673 (700457451H1), SEQ ID NO: 5915 (700457969H1), SEQ ID NO: 5108 (700456369H1), SEQ ID NO: 4115 (700454443H1), SEQ ID NO: 4518 (700455174H1), SEQ ID NO: 4359 (700454913H1), SEQ ID NO: 5493 (700457130H1), SEQ ID NO: 4057 (700454344H1), SEQ ID NO: 4975 (700456105H1), SEQ ID NO: 3803 (700453835H1), SEQ ID NO: 3799 (700453827H1), SEQ ID NO: 4953 (700456066H1), SEQ ID NO: 5672 (700457449H1), SEQ ID NO: 4290 (700454767H1), SEQ ID NO: 3742 (700453733H1), SEQ ID NO: 6290 (700458825H1), SEQ ID NO: 6248 (700458643H1). These selected cdps represent unique, corn seedling-specific polynucleotides which are used to produce a seedling-specific profile of gene transcription, a transcript image.

The cdps are also used as a composition in methods to detect altered gene expression in inbred or hybrid plants. Such methods employ the cdps of the Sequence Listing, oligonucleotides, fragments, derivatives, or complementary sequences in hybridization technologies. The invention provides a method for detecting polynucleotides in a biological sample, the method comprising the steps of hybridizing a cdp to at least one of the nucleic acids in the biological sample, thereby forming a hybridization complex, and detecting the hybridization complex, wherein the presence of the complex correlates with the presence of the polynucleotide in the sample. An additional method provides for amplification of the nucleic acids of the biological sample prior to hybridization. The invention provides a method of screening a plurality of molecules for specific binding to a polynucleotide, the method comprising the steps of providing the plurality of molecules; combining the polynucleotide with each of the plurality of molecules for a time sufficient to allow binding under suitable conditions; and detecting binding of the polynucleotide to each of the plurality of molecules, thereby identifying the molecules which specifically bind the polynucleotide.

The invention further provides a method for recovering a regulatory element, the method comprising the steps of designing oligomers to at least one of the cdps, combining the oligomers with a DNA library under appropriate conditions to amplify the cdp, comparing the cdp with the amplified sequence to identify overlapping areas, identifying additional sequence beyond the overlapping areas, and repeating steps a) through d) until the regulatory element is recovered. The regulatory element is a seedling-specific regulatory element which may be placed in an expression vector which is transformed into a corn plant. The regulatory element is of value in regulating the expression of introduced cdps.

The invention provides a purified corn seedling-derived polynucleotide capable of expressing a corn seedling-derived polypeptide. In one embodiment, the corn seedling-derived polynucleotide is contained within an expression vector. In a second embodiment, the expression vector is contained within a host cell. The invention also provides a method for producing a corn seedling-derived polypeptide, said method comprising the steps of culturing the host cells containing the seedling-derived polynucleotide under conditions suitable for the expression of a corn seedling-derived polypeptide, and recovering the corn seedling-derived polypeptide from the cell culture.

The invention provides a purified corn seedling-derived polypeptide (CDP) encoded by at least one of the cdps of the Sequence Listing. The invention also provides an anti-CDP antibody specific for a purified polypeptide encoded by the cdp. Such antibodies may be used for diagnostic purposes for the detection of CDPs in specific plant cells.

The invention further provides a method for identifying a test compound which specifically binds the CDP, the method comprising the steps of providing a test compound, combining the CDP with the test compound under suitable conditions for a time sufficient to allow binding, and detecting CDP binding to the test compound.

### DESCRIPTION OF THE SEQUENCE LISTING AND TABLES

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

The Sequence Listing is a compilation of nucleic acid sequences obtained by sequencing clone inserts or isolates of two corn seedling cDNA libraries. Each sequence is identified by a sequence identification number (SEQ ID NO) and by an Incyte Clone number. TABLES 1 and 2 are compilations of Incyte Clones arranged as described below.

TABLE 1 lists homologous isolates from the two corn seedling cDNA libraries prepared as described in the Examples. The first column contains Incyte Clone numbers. The Incyte Clone number provides a cross reference to the Sequence Listing. The second column contains a relevant GenBank identification number. The third and fourth columns represent product and log-likelihood scores (Karlin, S. and S. F. Altschul (1993) Proc. Nat. Acad. Sci. 90:5873-5877). The fifth column refers to the particular database and release of GenBank against which the Incyte Clone was searched and in which a related sequence was found. The sequences of this invention were compared to sequences in the GenBank plant, eukaryotic and protein databases, and most isolates list homology to sequences in those databases. The last column contains a description of the referenced GenBank sequence.

TABLE 2 is a compilation representing corn seedling-specific gene activity as illustrated by sets of clustered or related sequences. Each cluster disclosed in the table contains unique or homologous sequences that are specific to the two corn seedling cDNA libraries. The clones in a cluster may contain overlapping sequences or they may be related to or overlap a common reference sequence. Clusters are compiled by naming, matching, and counting all copies of the cdp. The minimum number of clones required to define a cluster is two; clusters containing two clones are found at the bottom of the table. Some clusters are characterized further by the homology of one or more sequences to a reference sequence which has a GenBank identifier (g) and description. Homologous sequences are more fully described in TABLE 1.

### DETAILED DESCRIPTION OF THE INVENTION

Before the nucleic acid sequences and methods are presented, it is to be understood that this invention is not limited to the particular methodologies, protocols, cell lines, vectors, and reagents described. Although particular embodiments are described, methods and materials similar or equivalent to these embodiments may be used to practice the invention. The preferred methods, devices, and materials set forth are not intended to limit the scope of the invention which is limited only by the appended claims.

The singular forms “a”, “an”, and “the” include plural reference unless the context clearly dictates otherwise. All technical and scientific terms have the meanings commonly understood by one of ordinary skill in the art. All publications are incorporated by reference for the purpose of describing and disclosing the cell lines, vectors, and methodologies which are presented and which might be used in connection with the invention. Nothing in the specification is to be construed as an admission that the invention is not entitled to antedate such disclosure by virtue of prior invention.

Definitions

As used herein, the lower case “cdp” refers to a nucleic acid sequence, while the upper case “CDP” refers to an amino acid sequence. A “full-length” cdp refers to a nucleic acid sequence containing the entire coding region of a gene endogenously expressed in corn.

“Adjuvants” are materials such as Freund's, mineral gels (aluminum hydroxide), and surface active substances (lysolecithin, pluronic polyols, polyanions, peptides, oil emulsions, keyhole limpet hemocyanin (KLH; Sigma-Aldrich, St. Louis Mo.), and dinitrophenol) which may be administered to increase a host's immunological response.

“Allele” refers to an alternative form of a nucleic acid sequence. Alleles result from a “mutation”, and any given genome may have none, one, or many allelic forms. Mutations which give rise to alleles are ascribed to deletions, additions or substitutions of nucleotides. Each of these types of changes may occur alone, or in combination with the others, one or more times in a given nucleic acid sequence. The present invention encompasses allelic cdps.

“Amino acid sequence” refers to an oligopeptide, a peptide, a polypeptide, or a protein of either natural or synthetic origin. The amino acid sequence is not limited to the complete, endogenous amino acid sequence and may be a portion, epitope, variant, or derivative of a protein expressed by a corn nucleic acid sequence.

“Amplification” refers to the production of additional copies of a sequence and is carried out using polymerase chain reaction (PCR) technologies well known in the art.

“Antisense” refers to nucleic acid sequences which are complementary to a specific DNA or RNA sequence. The antisense strand (negative or 3′-5′) is that nucleic acid strand that is complementary to the sense strand (positive or 5′-3′).

“Biologically active” refers to a peptide having a structural, regulatory biochemical, or immunological function of a naturally occurring peptide.

“Complementary” refers to the natural association of nucleic acid sequences by base-pairing (A-G-T pairs with its complement T-C-A). Complementarity between two single-stranded molecules may be partial or complete. The degree to which two sequences are complementary affects the efficiency of hybridization and amplification reactions.

“Derivative” refers to the chemical modification of a nucleic acid sequence by replacement of hydrogen by an alkyl, acyl, amino, or other group.

“Homology” refers to sequence similarity either between a reference nucleic acid sequence and at least a fragment of a cdp or between a reference amino acid sequence and a portion of a CDP.

“Hybridization” refers to the process by which a strand of nucleic acid joins with a complementary strand through base pairing.

“Immunogenic” defines the capability of a natural, recombinant or synthetic oligopeptide, or polypeptide, to induce antibody production in appropriate animals or cells.

“Labeling” refers to the covalent or noncovalent joining of a polynucleotide, polypeptide or antibody with a reporter molecule that provides for a detectable and often measurable signal.

“Linkers” are short stretches of nucleotide sequence which may be added onto a vector or a cdp to create restriction endonuclease sites for ease in cloning. “Polylinkers” are engineered to include multiple restriction enzyme sites and provide for the use of both those enzymes which leave 5′ and 3′ overhangs (such as BamHI, EcoRI, and HindIII) or which provide a blunt end (such as EcoRV, SnaBI, and StuI).

“Naturally occurring” refers to an endogenous polynucleotide or polypeptide that may be isolated from viruses or prokaryotic or eukaryotic cells.

“Nucleic acid sequence” refers to an oligomer, oligonucleotide, nucleotide or polynucleotide, and its fragments, and to DNA or RNA of genomic or synthetic origin which may be single- or double-stranded and represent the sense or complementary (antisense) strand.

“Oligomer” refers to a nucleic acid sequence of at least about 10 nucleotides and as many as about 60 nucleotides, preferably about 15 to 30 nucleotides, that may be used as a primer or amplimer in amplification technologies. Oligomers are usually chemically synthesized.

“Peptide nucleic acid” (PNA) refers to an oligomer of at least six nucleotides to which an amino acid residue, such as lysine, and an amino group have been added. PNAs, also designated antigene agents, stop transcript elongation by binding to their complementary strand of nucleic acid.

“Plant sample” refers to a cell, chromosomes isolated from a cell, genomic DNA, RNA, or cDNA in solution or bound to a substrate; an extract from plant cells, a cleared tissue, a blot or imprint from the cut edge of a plant part, or the like.

A “portion” of an CDP may be selected based upon retention of biological or immunological characteristics shared with naturally occurring polypeptides derived from corn seedling. For example, an antigenic portion of a CDP may be used to induce antibody in an appropriate host.

“Post-translational modification” of a CDP may involve lipidation, glycosylation, phosphorylation, acetylation, racemization, proteolytic cleavage, and the like. These processes may occur synthetically or biochemically. Biochemical modifications will vary by cell type depending on the enzymatic milieu and the CDP.

“Probe” refers to cdps or fragments thereof, which are used to detect identical, allelic or related nucleic acid sequences.

“Purified” refers to molecules, either polynucleotides or polypeptides that are isolated or separated from their natural environment and are at least 60% free, preferably 75% free, and most preferably 90% free from other compounds with which they are naturally associated.

“Regulatory element” refers to a nucleic acid sequence from nontranslated regions of a gene such as enhancers, promoters, introns, and 3′ untranslated regions which interact with host proteins to carry out transcription or translation.

“Reporter” molecules are chemical or biochemical moieties used for labeling a nucleic acid, an amino acid, or an antibody. They include radionuclides; enzymes; fluorescent, chemiluminescent, or chromogenic agents; substrates; cofactors; inhibitors; magnetic particles; and the like.

“Substrate” refers to any suitable rigid or semi-rigid support including membranes, filters, chips, slides, wafers, fibers, magnetic or nonmagnetic beads, gels, capillaries or other tubing, plates, polymers, and microparticles. The substrate can have a variety of surface forms, such as wells, trenches, pins, channels and pores, to which the polynucleotides are bound.

“Transformation” refers to a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed. Transformants include stably transformed cells in which the inserted DNA is capable of replication either as an autonomously replicating plasmid or as part of the host chromosome, as well as cells which transiently express the inserted DNA or RNA.

“Variant” refers to an amino acid sequence which differs from another sequence by at least one amino acid. The variant may have “conservative” changes (e.g. replacement of leucine with isoleucine), which does not affect structural or chemical properties; or more rarely, “nonconservative” changes (e.g. replacement of glycine with tryptophan), which may affect structural and/or chemical properties.

THE INVENTION

In a particular embodiment, mRNA was isolated from corn seedling and used to construct the SATMON012 and SATMON029 cDNA libraries. The invention relates to nucleic acid sequences comprising corn seedling-derived polynucleotides (cdps) as presented in the Sequence Listing and to the use of these nucleic acid sequences. A “corn seedling-derived polynucleotide” refers to a cdp which may be naturally occurring, recombinant, synthetic, or semi-synthetic. A subset of clustered seedling-specific cdps is given in TABLE 2. The cdps may be used to identify, isolate, or extend identical or related corn seedling nucleic acid sequences from DNA libraries for the purpose of producing an entire coding region or recovering a regulatory element. The cdps may also be used in nucleic acid hybridization or amplification technologies to follow expression of desirable traits through plant breeding programs. The present invention provides for expression vectors and host cells containing nucleic acid sequences that encode CDPs or portions thereof and regulatory elements obtained using methods described herein. The CDPs may possess biological or immunological activity, or both. The invention provides for the use of purified CDPs to induce antibodies for diagnostic use and to identify test compounds which specifically bind the CDP.

Specifically, the present invention relates to the following subset of unique and seedling-specific cdps whose transcripts occurred more than once in the two corn seedling cDNA library. They are SEQ ID NO: 3537 (700162463H1), SEQ ID NO: 1161 (700158211H1), SEQ ID NO: 949 (700157856H1), SEQ ID NO: 3199 (700161822H1), SEQ ID NO: 1744 (700159434H1), SEQ ID NO: 1183 (700158258H1), SEQ ID NO: 377 (700156836H1), SEQ ID NO: 2501 (700160693H1), SEQ ID NO: 2527 (700160740H1), SEQ ID NO: 698 (700157394H1), SEQ ID NO: 2335 (700160435H1), SEQ ID NO: 1988 (700159867H1), SEQ ID NO: 2583 (700160844H1), SEQ ID NO: 585 (700157190H1), SEQ ID NO: 237 (700156608H1), SEQ ID NO: 2719 (700161045H1), SEQ ID NO: 266 (700156661H1), SEQ ID NO: 3021 (700161544H1), SEQ ID NO: 2040 (700159955H1), SEQ ID NO: 2224 (700160253H1), SEQ ID NO: 2761 (700161134H1), SEQ ID NO: 1166 (700158229H1), SEQ ID NO: 1164 (700158222H1), SEQ ID NO: 2479 (700160669H1), SEQ ID NO: 215 (700156563H1), SEQ ID NO: 2448 (700160623H1), SEQ ID NO: 2207 (700160227H1), SEQ ID NO: 2626 (700160906H1), SEQ ID NO: 1339 (700158532H1), SEQ ID NO: 2726 (700161054H1), SEQ ID NO: 336 (700156768H1), SEQ ID NO: 1963 (700159826H1), SEQ ID NO: 2285 (700160357H1), SEQ ID NO: 1969 (700159839H1), SEQ ID NO: 471 (700156989H1), SEQ ID NO: 3118 (700161689H1), SEQ ID NO: 2475 (700160665H1), SEQ ID NO: 2731 (700161064H1), SEQ ID NO: 1695 (700159330H1), SEQ ID NO: 35 (700142454H1), SEQ ID NO: 79 (700142522H1), SEQ ID NO: 2086 (700160030H1), SEQ ID NO: 2000 (700159882H1), SEQ ID NO: 1001 (700157954H1), SEQ ID NO: 1302 (700158460H1), SEQ ID NO: 1205 (700158305H1), SEQ ID NO: 2849 (700161279H1), SEQ ID NO: 2138 (700160112H1), SEQ ID NO: 224 (700156580H1), SEQ ID NO: 385 (700156847H1), SEQ ID NO: 3541 (700162468H1), SEQ ID NO: 3526 (700162448H1), SEQ ID NO: 2137 (700160111H1), SEQ ID NO: 1197 (700158287H1), SEQ ID NO: 2109 (700160067H1), SEQ ID NO: 1902 (700159726H1), SEQ ID NO: 2578 (700160832H1), SEQ ID NO: 1044 (700158022H1), SEQ ID NO: 956 (700157865H1), SEQ ID NO: 149 (700156439H1), SEQ ID NO: 1548 (700159028H1), SEQ ID NO: 3030 (700161557H1), SEQ ID NO: 2128 (700160091H1), SEQ ID NO: 3025 (700161550H1), SEQ ID NO: 195 (700156524H1), SEQ ID NO: 1385 (700158604H1), SEQ ID NO: 1091 (700158090H1), SEQ ID NO: 1517 (700158965H1), SEQ ID NO: 1439 (700158806H1), SEQ ID NO: 2755 (700161125H1), SEQ ID NO: 1449 (700158823H1), SEQ ID NO: 107 (700142566H1), SEQ ID NO: 2916 (700161388H1), SEQ ID NO: 3160 (700161766H1), SEQ ID NO: 3001 (700161515H1), SEQ ID NO: 1940 (700159780H1), SEQ ID NO: 2918 (700161390H1), SEQ ID NO: 426 (700156915H1), SEQ ID NO: 1024 (700157988H1), SEQ ID NO: 2468 (700160654H1), SEQ ID NO: 1886 (700159694H1), SEQ ID NO: 1160 (700158208H1), SEQ ID NO: 411 (700156889H1), SEQ ID NO: 1570 (700159068H1), SEQ ID NO: 3371 (700162137H1), SEQ ID NO: 1942 (700159783H1), SEQ ID NO: 3132 (700161719H1), SEQ ID NO: 138 (700156425H1), SEQ ID NO: 2870 (700161314H1), SEQ ID NO: 2624 (700160904H1), SEQ ID NO: 3518 (700162435H1), SEQ ID NO: 2931 (700161413H1), SEQ ID NO: 1649 (700159219H1), SEQ ID NO: 2477 (700160667H1), SEQ ID NO: 1513 (700158949H1), SEQ ID NO: 3340 (700162062H1), SEQ ID NO: 884 (700157746H1), SEQ ID NO: 1701 (700159340H1), SEQ ID NO: 2480 (700160671H1), SEQ ID NO: 938 (700157837H1), SEQ ID NO: 2834 (700161257H1), SEQ ID NO: 1020 (700157981H1), SEQ ID NO: 2272 (700160340H1), SEQ ID NO: 2055 (700159981H1), SEQ ID NO: 489 (700157022H1), SEQ ID NO: 1294 (700158449H1), SEQ ID NO: 1597 (700159125H1), SEQ ID NO: 1172 (700158240H1), SEQ ID NO: 1243 (700158366H1), SEQ ID NO: 2482 (700160674H1), SEQ ID NO: 2544 (700160768H1), SEQ ID NO: 2862 (700161296H1), SEQ ID NO: 2704 (700161024H1), SEQ ID NO: 2369 (700160492H1), SEQ ID NO: 3242 (700161894H1), SEQ ID NO: 2948 (700161439H1), SEQ ID NO: 1315 (700158484H1), SEQ ID NO: 2700 (700161016H1), SEQ ID NO: 1436 (700158695H1), SEQ ID NO: 1996 (700159877H1), SEQ ID NO: 172 (700156474H1), SEQ ID NO: 1787 (700159515H1), SEQ ID NO: 1929 (700159766H1), SEQ ID NO: 3601 (700162584H1), SEQ ID NO: 1615 (700159167H1), SEQ ID NO: 852 (700157686H1), SEQ ID NO: 2267 (700160335H1), SEQ ID NO: 2887 (700161344H1), SEQ ID NO: 803 (700157615H1), SEQ ID NO: 1878 (700159679H1), SEQ ID NO: 2026 (700159930H1), SEQ ID NO: 1250 (700158376H1), SEQ ID NO: 573 (700157172H1), SEQ ID NO: 105 (700142562H1), SEQ ID NO: 2205 (700160225H1), SEQ ID NO: 2389 (700160522H1), SEQ ID NO: 2876 (700161326H1), SEQ ID NO: 62 (700142492H1), SEQ ID NO: 2469 (700160658H1), SEQ ID NO: 1206 (700158310H1), SEQ ID NO: 666 (700157334H1), SEQ ID NO: 179 (700156486H1), SEQ ID NO: 1378 (700158590H1), SEQ ID NO: 3316 (700162025H1), SEQ ID NO: 578 (700157181H1), SEQ ID NO: 2433 (700160601H1), SEQ ID NO: 865 (700157712H1), SEQ ID NO: 1726 (700159386H1), SEQ ID NO: 3165 (700161772H1), SEQ ID NO: 1409 (700158643H1), SEQ ID NO: 2183 (700160186H1), SEQ ID NO: 2977 (700161476H1), SEQ ID NO: 1985 (700159863H1), SEQ ID NO: 768 (700157547H1), SEQ ID NO: 1499 (700158924H1), SEQ ID NO: 1404 (700158632H1), SEQ ID NO: 374 (700156831H1), SEQ ID NO: 1908 (700159733H1), SEQ ID NO: 1405 (700158636H1), SEQ ID NO: 1543 (700159020H1), SEQ ID NO: 2139 (700160115H1), SEQ ID NO: 1258 (700158387H1), SEQ ID NO: 3344 (700162066H1), SEQ ID NO: 3580 (700162539H1), SEQ ID NO: 1830 (700159602H1), SEQ ID NO: 3531 (700162455H1), SEQ ID NO: 515 (700157065H1), SEQ ID NO: 2723 (700161051H1), SEQ ID NO: 191 (700156519H1), SEQ ID NO: 3259 (700161925H1), SEQ ID NO: 622 (700157251H1), SEQ ID NO: 68 (700142503H1), SEQ ID NO: 32 (700142451H1), SEQ ID NO: 80 (700142523H1), SEQ ID NO: 2225 (700160259H1), SEQ ID NO: 37 (700142458H1), SEQ ID NO: 559 (700157147H1), SEQ ID NO: 581 (700157185H1), SEQ ID NO: 3042 (700161571H1), SEQ ID NO: 2789 (700161181H1), SEQ ID NO: 2692 (700161003H1), SEQ ID NO: 1493 (700158915H1), SEQ ID NO: 2431 (700160595H1), SEQ ID NO: 270 (700156667H1), SEQ ID NO: 2010 (700159905H1), SEQ ID NO: 3401 (700162185H1), SEQ ID NO: 3501 (700162392H1), SEQ ID NO: 307 (700156727H1), SEQ ID NO: 2830 (700161249H1), SEQ ID NO: 3464 (700162321H1), SEQ ID NO: 1034 (700158010H1), SEQ ID NO: 2573 (700160826H1), SEQ ID NO: 1657 (700159236H1), SEQ ID NO: 1646 (700159214H1), SEQ ID NO: 198 (700156531H1), SEQ ID NO: 151 (700156441H1), SEQ ID NO: 3257 (700161923H1), SEQ ID NO: 3553 (700162485H1), SEQ ID NO: 1839 (700159614H1), SEQ ID NO: 3056 (700161591H1), SEQ ID NO: 3320 (700162032H1), SEQ ID NO: 629 (700157262H1), SEQ ID NO: 1889 (700159703H1), SEQ ID NO: 2628 (700160908H1), SEQ ID NO: 1670 (700159272H1), SEQ ID NO: 1274 (700158421H1), SEQ ID NO: 1039 (700158016H1), SEQ ID NO: 1975 (700159850H1), SEQ ID NO: 2951 (700161442H1), SEQ ID NO: 1042 (700158020H1), SEQ ID NO: 1521 (700158980H1), SEQ ID NO: 1119 (700158143H1), SEQ ID NO: 277 (700156677H1), SEQ ID NO: 519 (700157072H1), SEQ ID NO: 1422 (700158672H1), SEQ ID NO: 2315 (700160405H1), SEQ ID NO: 922 (700157810H1), SEQ ID NO: 1175 (700158245H1), SEQ ID NO: 1763 (700159461H1), SEQ ID NO: 701 (700157405H1), SEQ ID NO: 2540 (700160761H1), SEQ ID NO: 1157 (700158204H1), SEQ ID NO: 576 (700157177H1), SEQ ID NO: 356 (700156801H1), SEQ ID NO: 1375 (700158585H1), SEQ ID NO: 3146 (700161738H1), SEQ ID NO: 3150 (700161744H1), SEQ ID NO: 1464 (700158854H1), SEQ ID NO: 2963 (700161457H1), SEQ ID NO: 2213 (700160236H1), SEQ ID NO: 1128 (700158158H1), SEQ ID NO: 3528 (700162450H1), SEQ ID NO: 1730 (700159395H1), SEQ ID NO: 3168 (700161775H1), SEQ ID NO: 918 (700157804H1), SEQ ID NO: 1775 (700159482H1), SEQ ID NO: 2676 (700160978H1), SEQ ID NO: 1567 (700159064H1), SEQ ID NO: 2412 (700160563H1), SEQ ID NO: 1003 (700157957H1), SEQ ID NO: 109 (700142571H1), SEQ ID NO: 1589 (700159110H1), SEQ ID NO: 508 (700157053H1), SEQ ID NO: 354 (700156792H1), SEQ ID NO: 505 (700157045H1), SEQ ID NO: 1307 (700158470H1), SEQ ID NO: 600 (700157219H1), SEQ ID NO: 1121 (700158145H1), SEQ ID NO: 1377 (700158588H1), SEQ ID NO: 2710 (700161034H1), SEQ ID NO: 1843 (700159620H1), SEQ ID NO: 998 (700157949H1), SEQ ID NO: 3564 (700162513H1), SEQ ID NO: 2708 (700161029H1), SEQ ID NO: 796 (700157593H1), SEQ ID NO: 1346 (700158541H1), SEQ ID NO: 2594 (700160860H1), SEQ ID NO: 898 (700157768H1), SEQ ID NO: 3384 (700162163H1), SEQ ID NO: 1552 (700159036H1), SEQ ID NO: 1554 (700159038H1), SEQ ID NO: 1712 (700159356H1), SEQ ID NO: 3094 (700161653H1), SEQ ID NO: 3525 (700162447H1), SEQ ID NO: 1858 (700159650H1), SEQ ID NO: 3428 (700162243H1), 10 SEQ ID NO: 1997 (700159879H1), SEQ ID NO: 2451 (700160628H1), SEQ ID NO: 429 (700156922H1), SEQ ID NO: 1906 (700159731H1), SEQ ID NO: 2660 (700160954H1), SEQ ID NO: 1557 (700159046H1), SEQ ID NO: 2672 (700160972H1), SEQ ID NO: 1066 (700158055H1), SEQ ID NO: 3449 (700162285H1), SEQ ID NO: 1693 (700159327H1), SEQ ID NO: 601 (700157222H1), SEQ ID NO: 3527 (700162449H1), SEQ ID NO: 85 (700142532H1), SEQ ID NO: 2062 (700159989H1), SEQ ID NO: 1862 (700159656H1), SEQ ID NO: 3544 (700162472H1), SEQ ID NO: 279 (700156681H1), SEQ ID NO: 2842 (700161267H1), SEQ ID NO: 1210 (700158315H1), SEQ ID NO: 2456 (700160640H1), SEQ ID NO: 2620 (700160893H1), SEQ ID NO: 724 (700157446H1), SEQ ID NO: 615 (700157240H1), SEQ ID NO: 1694 (700159328H1), SEQ ID NO: 500 (700157039H1), 20 SEQ ID NO: 2781 (700161170H1), SEQ ID NO: 2909 (700161381H1), SEQ ID NO: 436 (700156929H1), SEQ ID NO: 1036 (700158013H1), SEQ ID NO: 1736 (700159416H1), SEQ ID NO: 2569 (700160819H1), SEQ ID NO: 3313 (700162020H1), SEQ ID NO: 1073 (700158065H1), SEQ ID NO: 1990 (700159869H1), SEQ ID NO: 88 (700142539H1), SEQ ID NO: 1704 (700159347H1), SEQ ID NO: 1158 (700158205H1), SEQ ID NO: 3586 (700162548H1), SEQ ID NO: 1901 (700159724H1), SEQ ID NO: 1994 (700159875H1), SEQ ID NO: 3142 (700161734H1), SEQ ID NO: 2907 (700161378H1), SEQ ID NO: 1937 (700159776H1), SEQ ID NO: 2767 (700161143H1), SEQ ID NO: 2032 (700159941H1), SEQ ID NO: 3099 (700161661H1), SEQ ID NO: 2828 (700161245H1), SEQ ID NO: 437 (700156933H1), SEQ ID NO: 2985 (700161490H1), SEQ ID NO: 2499 (700160691H1), 30 SEQ ID NO: 2336 (700160436H1), SEQ ID NO: 1074 (700158066H1), SEQ ID NO: 2073 (700160007H1), SEQ ID NO: 2956 (700161448H1), SEQ ID NO: 1089 (700158086H1), SEQ ID NO: 2195 (700160211H1), SEQ ID NO: 1741 (700159425H1), SEQ ID NO: 607 (700157229H1), SEQ ID NO: 2840 (700161265H1), SEQ ID NO: 2649 (700160942H1), SEQ ID NO: 1221 (700158332H1), SEQ ID NO: 1813 (700159568H1), SEQ ID NO: 1805 (700159552H1), SEQ ID NO: 285 (700156691H1), SEQ ID NO: 294 (700156709H1), SEQ ID NO: 3291 (700161979H1), SEQ ID NO: 2634 (700160921H1), SEQ ID NO: 3039 (700161567H1), SEQ ID NO: 3054 (700161589H1), SEQ ID NO: 453 (700156964H1), SEQ ID NO: 2681 (700160985H1), SEQ ID NO: 937 (700157836H1), SEQ ID NO: 1703 (700159346H1), SEQ ID NO: 2829 (700161247H1), SEQ ID NO: 3120 (700161693H1), SEQ ID NO: 552 (700157137H1), SEQ ID NO: 1431 (700158688H1), SEQ ID NO: 1806 (700159554H1), SEQ ID NO: 1814 (700159570H1), SEQ ID NO: 3308 (700162013H1), SEQ ID NO: 167 (700156463H1), SEQ ID NO: 2059 (700159986H1), SEQ ID NO: 242 (700156618H1), SEQ ID NO: 1256 (700158383H1), SEQ ID NO: 744 (700157489H1), SEQ ID NO: 1895 (700159716H1), SEQ ID NO: 440 (700156938H1), SEQ ID NO: 3217 (700161849H1), SEQ ID NO: 2831 (700161251H1), SEQ ID NO: 1015 (700157975H1), SEQ ID NO: 1366 (700158568H1), SEQ ID NO: 314 (700156740H1), SEQ ID NO: 2188 (700160195H1), SEQ ID NO: 84 (700142528H1), SEQ ID NO: 3069 (700161614H1), SEQ ID NO: 2952 (700161443H1), SEQ ID NO: 53 (700142478H1), SEQ ID NO: 1778 (700159492H1), SEQ ID NO: 1881 (700159685H1), SEQ ID NO: 1907 (700159732H1), SEQ ID NO: 614 (700157239H1), SEQ ID NO: 3395 (700162178H1), SEQ ID NO: 1312 (700158481H1), SEQ ID NO: 243 (700156621H1), SEQ ID NO: 1131 (700158163H1), SEQ ID NO: 678 (700157359H1), SEQ ID NO: 1506 (700158934H1), SEQ ID NO: 2643 (700160932H1), SEQ ID NO: 2593 (700160857H1), SEQ ID NO: 142 (700156430H1), SEQ ID NO: 2371 (700160494H1), SEQ ID NO: 925 (700157817H1), SEQ ID NO: 3041 (700161570H1), SEQ ID NO: 3050 (700161583H1), SEQ ID NO: 3079 (700161637H1), SEQ ID NO: 1245 (700158369H1), SEQ ID NO: 2309 (700160391H1), SEQ ID NO: 2713 (700161037H1), SEQ ID NO: 1289 (700158440H1), SEQ ID NO: 635 (700157268H1), SEQ ID NO: 653 (700157309H1), SEQ ID NO: 244 (700156623H1), SEQ ID NO: 338 (700156771H1), SEQ ID NO: 1406 (700158637H1), SEQ ID NO: 2941 (700161430H1), SEQ ID NO: 2516 (700160725H1), SEQ ID NO: 1945 (700159791H1), SEQ ID NO: 1485 (700158902H1), SEQ ID NO: 3045 (700161576H1), SEQ ID NO: 1271 (700158417H1), SEQ ID NO: 2786 (700161176H1), SEQ ID NO: 156 (700156447H1), SEQ ID NO: 1367 (700158574H1), SEQ ID NO: 3169 (700161776H1), SEQ ID NO: 2076 (700160012H1), SEQ ID NO: 3253 (700161917H1), SEQ ID NO: 950 (700157857H1), SEQ ID NO: 1237 (700158356H1), SEQ ID NO: 2063 (700159990H1), SEQ ID NO: 3574 (700162527H1), SEQ ID NO: 580 (700157183H1), SEQ ID NO: 2034 (700159943H1), SEQ ID NO: 892 (700157757H1), SEQ ID NO: 1096 (700158104H1), SEQ ID NO: 1883 (700159691H1), SEQ ID NO: 2966 (700161460H1), SEQ ID NO: 1632 (700159193H1), SEQ ID NO: 1811 (700159565H1), SEQ ID NO: 1803 (700159549H1), SEQ ID NO: 2390 (700160523H1), SEQ ID NO: 2329 (700160426H1), SEQ ID NO: 2835 (700161259H1), SEQ ID NO: 1428 (700158681H1), SEQ ID NO: 2016 (700159915H1), SEQ ID NO: 3487 (700162367H1), SEQ ID NO: 2699 (700161014H1), SEQ ID NO: 2694 (700161006H1), SEQ ID NO: 163 (700156456H1), SEQ ID NO: 2302 (700160381H1), SEQ ID NO: 23 (700142438H1), SEQ ID NO: 1720 (700159376H1), SEQ ID NO: 1519 (700158975H1), SEQ ID NO: 569 (700157166H1), SEQ ID NO: 3015 (700161536H1), SEQ ID NO: 521 (700157076H1), SEQ ID NO: 736 (700157474H1), SEQ ID NO: 626 (700157256H1), SEQ ID NO: 1846 (700159627H1), SEQ ID NO: 1642 (700159209H1), SEQ ID NO: 2391 (700160526H1), SEQ ID NO: 2410 (700160559H1), SEQ ID NO: 2061 (700159988H1), SEQ ID NO: 413 (700156891H1), SEQ ID NO: 1072 (700158064H1), SEQ ID NO: 1038 (700158015H1), SEQ ID NO: 1854 (700159641H1), SEQ ID NO: 143 (700156431H1), SEQ ID NO: 81 (700142524H1), SEQ ID NO: 3111 (700161679H1), SEQ ID NO: 2806 (700161213H1), SEQ ID NO: 139 (700156426H1), SEQ ID NO: 43 (700142466H1), SEQ ID NO: 185 (700156495H1), SEQ ID NO: 2524 (700160737H1), SEQ ID NO: 64 (700142494H1), SEQ ID NO: 3171 (700161778H1), SEQ ID NO: 2707 (700161028H1), SEQ ID NO: 2033 (700159942H1), SEQ ID NO: 639 (700157276H1), SEQ ID NO: 532 (700157093H1), SEQ ID NO: 3155 (700161756H1), SEQ ID NO: 1338 (700158531H1), SEQ ID NO: 575 (700157176H1), SEQ ID NO: 1177 (700158248H1), SEQ ID NO: 1099 (700158107H1), SEQ ID NO: 1861 (700159654H1), SEQ ID NO: 1832 (700159606H1), SEQ ID NO: 829 (700157650H1), SEQ ID NO: 878 (700157737H1), SEQ ID NO: 1443 (700158812H1), SEQ ID NO: 329 (700156761H1), SEQ ID NO: 3227 (700161866H1), SEQ ID NO: 851 (700157685H1), SEQ ID NO: 2728 (700161056H1), SEQ ID NO: 2854 (700161284H1), SEQ ID NO: 3547 (700162477H1), SEQ ID NO: 2393 (700160529H1), SEQ ID NO: 323 (700156754H1), SEQ ID NO: 1407 (700158638H1), SEQ ID NO: 187 (700156506H1), SEQ ID NO: 441 (700156940H1), SEQ ID NO: 1904 (700159728H1), SEQ ID NO: 1386 (700158605H1), SEQ ID NO: 3323 (700162037H1), SEQ ID NO: 2576 (700160830H1), SEQ ID NO: 1283 (700158434H1), SEQ ID NO: 2048 (700159970H1), SEQ ID NO: 2123 (700160085H1), SEQ ID NO: 2305 (700160385H1), SEQ ID NO: 1191 (700158278H1), SEQ ID NO: 1420 (700158669H1), SEQ ID NO: 335 (700156767H1), SEQ ID NO: 2889 (700161346H1), SEQ ID NO: 798 (700157601H1), SEQ ID NO: 976 (700157911H1), SEQ ID NO: 104 (700142561H1), SEQ ID NO: 1052 (700158034H1), SEQ ID NO: 1347 (700158542H1), SEQ ID NO: 1653 (700159226H1), SEQ ID NO: 958 (700157868H1), SEQ ID NO: 2252 (700160313H1), SEQ ID NO: 2181 (700160184H1), SEQ ID NO: 971 (700157895H1), SEQ ID NO: 2492 (700160684H1), SEQ ID NO: 1043 (700158021H1), SEQ ID NO: 12 (700142422H1), SEQ ID NO: 592 (700157207H1), SEQ ID NO: 3163 (700161770H1), SEQ ID NO: 3331 (700162049H1), SEQ ID NO: 2591 (700160855H1), SEQ ID NO: 2153 (700160140H1), SEQ ID NO: 1917 (700159748H1), SEQ ID NO: 2824 (700161241H1), SEQ ID NO: 3407 (700162206H1), SEQ ID NO: 1453 (700158832H1), SEQ ID NO: 2747 (700161102H1), SEQ ID NO: 2801 (700161206H1), SEQ ID NO: 1558 (700159047H1), SEQ ID NO: 2888 (700161345H1), SEQ ID NO: 1681 (700159307H1), SEQ ID NO: 2330 (700160427H1), SEQ ID NO: 1607 (700159151H1), SEQ ID NO: 1635 (700159196H1), SEQ ID NO: 2115 (700160076H1), SEQ ID NO: 656 (700157319H1), SEQ ID NO: 888 (700157751H1), SEQ ID NO: 711 (700157424H1), SEQ ID NO: 2307 (700160387H1), SEQ ID NO: 145 (700156433H1), SEQ ID NO: 1734 (700159411H1), SEQ ID NO: 342 (700156776H1), SEQ ID NO: 1842 (700159619H1), SEQ ID NO: 1360 (700158558H1), SEQ ID NO: 799 (700157605H1), SEQ ID NO: 1759 (700159454H1), SEQ ID NO: 2486 (700160678H1), SEQ ID NO: 3194 (700161816H1), SEQ ID NO: 1457 (700158839H1), SEQ ID NO: 2666 (700160964H1), SEQ ID NO: 3509 (700162417H1), SEQ ID NO: 2430 (700160593H1), SEQ ID NO: 446 (700156951H1), SEQ ID NO: 3273 (700161948H1), SEQ ID NO: 822 (700157642H1), SEQ ID NO: 1981 (700159857H1), SEQ ID NO: 1146 (700158184H1), SEQ ID NO: 3594 (700162570H1), SEQ ID NO: 2179 (700160180H1), SEQ ID NO: 1195 (700158285H1), SEQ ID NO: 3489 (700162376H1), SEQ ID NO: 2192 (700160204H1), SEQ ID NO: 3023 (700161547H1), SEQ ID NO: 363 (700156813H1), SEQ ID NO: 2498 (700160690H1), SEQ ID NO: 2491 (700160683H1), SEQ ID NO: 1875 (700159673H1), SEQ ID NO: 127 (700156407H1), SEQ ID NO: 1381 (700158596H1), SEQ ID NO: 1630 (700159187H1), SEQ ID NO: 3325 (700162042H1), SEQ ID NO: 3444 (700162277H1), SEQ ID NO: 1705 (700159348H1), SEQ ID NO: 2533 (700160750H1), SEQ ID NO: 1986 (700159864H1), SEQ ID NO: 2348 (700160459H1), SEQ ID NO: 3019 (700161541H1), SEQ ID NO: 2557 (700160795H1), SEQ ID NO: 3212 (700161842H1), SEQ ID NO: 3202 (700161826H1), SEQ ID NO: 1745 (700159435H1), SEQ ID NO: 188 (700156511H1), SEQ ID NO: 964 (700157879H1), SEQ ID NO: 3470 (700162338H1), SEQ ID NO: 3603 (700162591H1), SEQ ID NO: 2500 (700160692H1), SEQ ID NO: 2775 (700161157H1), SEQ ID NO: 2778 (700161166H1), SEQ ID NO: 1740 (700159423H1), SEQ ID NO: 1576 (700159081H1), SEQ ID NO: 3508 (700162414H1), SEQ ID NO: 3484 (700162364H1), SEQ ID NO: 1054 (700158037H1), SEQ ID NO: 4316 (700454828H1), SEQ ID NO: 4594 (700455401H1), SEQ ID NO: 4100 (700454421H1), SEQ ID NO: 5528 (700457192H1), SEQ ID NO: 5760 (700457654H1), SEQ ID NO: 3797 (700453825H1), SEQ ID NO: 5257 (700456691H1), SEQ ID NO: 3788 (700453810H1), SEQ ID NO: 4097 (700454418H1), SEQ ID NO: 5731 (700457589H1), SEQ ID NO: 5009 (700456178H1), SEQ ID NO: 4972 (700456102H1), SEQ ID NO: 5423 (700457018H1), SEQ ID NO: 4519 (700455175H1), SEQ ID NO: 4729 (700455675H1), SEQ ID NO: 5640 (700457388H1), SEQ ID NO: 6092 (700458353H1), SEQ ID NO: 4157 (700454513H1), SEQ ID NO: 6103 (700458378H1), SEQ ID NO: 3821 (700453864H1), SEQ ID NO: 4968 (700456094H1), SEQ ID NO: 4292 (700454771H1), SEQ ID NO: 5425 (700457023H1), SEQ ID NO: 5154 (700456454H1), SEQ ID NO: 5445 (700457055H1), SEQ ID NO: 5146 (700456444H1), SEQ ID NO: 4643 (700455525H1), SEQ ID NO: 5267 (700456715H1), SEQ ID NO: 4380 (700454949H1), SEQ ID NO: 3883 (700453981H1), SEQ ID NO: 5847 (700457846H1), SEQ ID NO: 4889 (700455957H1), SEQ ID NO: 5239 (700456657H1), SEQ ID NO: 5072 (700456315H1), SEQ ID NO: 6018 (700458195H1), SEQ ID NO: 5205 (700456575H1), SEQ ID NO: 5826 (700457804H1), SEQ ID NO: 4475 (700455102H1), SEQ ID NO: 5429 (700457030H1), SEQ ID NO: 5452 (700457062H1), SEQ ID NO: 5453 (700457063H1), SEQ ID NO: 4131 (700454465H1), SEQ ID NO: 5538 (700457213H1), SEQ ID NO: 5540 (700457215H1), SEQ ID NO: 5433 (700457035H1), SEQ ID NO: 3984 (700454206H1), SEQ ID NO: 5369 (700456891H1), SEQ ID NO: 5766 (700457667H1), SEQ ID NO: 5699 (700457519H1), SEQ ID NO: 5698 (700457518H1), SEQ ID NO: 5907 (700457953H1), SEQ ID NO: 5829 (700457808H1), SEQ ID NO: 5872 (700457887H1), SEQ ID NO: 4247 (700454678H1), SEQ ID NO: 5225 (700456633H1), SEQ ID NO: 4823 (700455851H1), SEQ ID NO: 4083 (700454394H1), SEQ ID NO: 4084 (700454395H1), SEQ ID NO: 4030 (700454295H1), SEQ ID NO: 5636 (700457380H1), SEQ ID NO: 4784 (700455780H1), SEQ ID NO: 5925 (700457993H1), SEQ ID NO: 6119 (700458416H1), SEQ ID NO: 6124 (700458424H1), SEQ ID NO: 4463 (700455081H1), SEQ ID NO: 5554 (700457243H1), SEQ ID NO: 5497 (700457136H1), SEQ ID NO: 5602 (700457326H1), SEQ ID NO: 5651 (700457411H1), SEQ ID NO: 4522 (700455178H1), SEQ ID NO: 5647 (700457403H1), SEQ ID NO: 5426 (700457025H1), SEQ ID NO: 5395 (700456958H1), SEQ ID NO: 5422 (700457017H1), SEQ ID NO: 6127 (700458428H1), SEQ ID NO: 6121 (700458420H1), SEQ ID NO: 6128 (700458429H1), SEQ ID NO: 4915 (700456004H1), SEQ ID NO: 5183 (700456520H1), SEQ ID NO: 5179 (700456505H1), SEQ ID NO: 5480 (700457107H1), SEQ ID NO: 6070 (700458311H1), SEQ ID NO: 4263 (700454707H1), SEQ ID NO: 4325 (700454841H1), SEQ ID NO: 5552 (700457241H1), SEQ ID NO: 6241 (700458630H1), SEQ ID NO: 4853 (700455908H1), SEQ ID NO: 5088 (700456339H1), SEQ ID NO: 5082 (700456331H1), SEQ ID NO: 4419 (700455012H1), SEQ ID NO: 5131 (700456416H1), SEQ ID NO: 4417 (700455010H1), SEQ ID NO: 4137 (700454471H1), SEQ ID NO: 4786 (700455782H1), SEQ ID NO: 5221 (700456626H1), SEQ ID NO: 4095 (700454414H1), SEQ ID NO: 4453 (700455062H1), SEQ ID NO: 5253 (700456683H1), SEQ ID NO: 4588 (700455290H1), SEQ ID NO: 4456 (700455068H1), SEQ ID NO: 4428 (700455024H1), SEQ ID NO: 3825 (700453874H1), SEQ ID NO: 4686 (700455610H1), SEQ ID NO: 4711 (700455650H1), SEQ ID NO: 4067 (700454365H1), SEQ ID NO: 4436 (700455038H1), SEQ ID NO: 6010 (700458175H1), SEQ ID NO: 4256 (700454692H1), SEQ ID NO: 5474 (700457092H1), SEQ ID NO: 4656 (700455554H1), SEQ ID NO: 4832 (700455867H1), SEQ ID NO: 5744 (700457627H1), SEQ ID NO: 5226 (700456634H1), SEQ ID NO: 4529 (700455189H1), SEQ ID NO: 5471 (700457085H1), SEQ ID NO: 5451 (700457061H1), SEQ ID NO: 5828 (700457807H1), SEQ ID NO: 5771 (700457674H1), SEQ ID NO: 4957 (700456074H1), SEQ ID NO: 4985 (700456127H1), SEQ ID NO: 4981 (700456114H1), SEQ ID NO: 3971 (700454173H1), SEQ ID NO: 4060 (700454351H1), SEQ ID NO: 4734 (700455681H1), SEQ ID NO: 5136 (700456423H1), SEQ ID NO: 5680 (700457471H1), SEQ ID NO: 5980 (700458123H1), SEQ ID NO: 3940 (700454119H1), SEQ ID NO: 3866 (700453958H1), SEQ ID NO: 5861 (700457867H1), SEQ ID NO: 3836 (700453895H1), SEQ ID NO: 5458 (700457072H1), SEQ ID NO: 4366 (700454923H1), SEQ ID NO: 4211 (700454614H1), SEQ ID NO: 6273 (700458684H1), SEQ ID NO: 5875 (700457891H1), SEQ ID NO: 5140 (700456433H1), SEQ ID NO: 5986 (700458136H1), SEQ ID NO: 6262 (700458668H1), SEQ ID NO: 5247 (700456675H1), SEQ ID NO: 5721 (700457565H1), SEQ ID NO: 4531 (700455195H1), SEQ ID NO: 5577 (700457273H1), SEQ ID NO: 4399 (700454984H1), SEQ ID NO: 6072 (700458313H1), SEQ ID NO: 4442 (700455048H1), SEQ ID NO: 4078 (700454381H1), SEQ ID NO: 4751 (700455713H1), SEQ ID NO: 5790 (700457717H1), SEQ ID NO: 5406 (700456977H1), SEQ ID NO: 4532 (700455196H1), SEQ ID NO: 4012 (700454262H1), SEQ ID NO: 5556 (700457245H1), SEQ ID NO: 5262 (700456707H1), SEQ ID NO: 4900 (700455974H1), SEQ ID NO: 5290 (700456752H1), SEQ ID NO: 3957 (700454145H1), SEQ ID NO: 6291 (700458826H1), SEQ ID NO: 6292 (700458827H1), SEQ ID NO: 4639 (700455517H1), SEQ ID NO: 6137 (700458443H1), SEQ ID NO: 4367 (700454925H1), SEQ ID NO: 4467 (700455085H1), SEQ ID NO: 5473 (700457090H1), SEQ ID NO: 5482 (700457115H1), SEQ ID NO: 4266 (700454717H1), SEQ ID NO: 4272 (700454725H1), SEQ ID NO: 4356 (700454909H1), SEQ ID NO: 4135 (700454469H1), SEQ ID NO: 5259 (700456694H1), SEQ ID NO: 5736 (700457604H1), SEQ ID NO: 5114 (700456381H1), SEQ ID NO: 5656 (700457419H1), SEQ ID NO: 4512 (700455167H1), SEQ ID NO: 4460 (700455077H1), SEQ ID NO: 4553 (700455237H1), SEQ ID NO: 4373 (700454937H1), SEQ ID NO: 4400 (700454985H1), SEQ ID NO: 5379 (700456917H1), SEQ ID NO: 5678 (700457466H1), SEQ ID NO: 5746 (700457629H1), SEQ ID NO: 5152 (700456452H1), SEQ ID NO: 5133 (700456418H1), SEQ ID NO: 4908 (700455987H1), SEQ ID NO: 5650 (700457409H1), SEQ ID NO: 6060 (700458287H1), SEQ ID NO: 6011 (700458178H1), SEQ ID NO: 4271 (700454724H1), SEQ ID NO: 5489 (700457124H1), SEQ ID NO: 5762 (700457658H1), SEQ ID NO: 4068 (700454366H1), SEQ ID NO: 4075 (700454377H1), SEQ ID NO: 3910 (700454049H1), SEQ ID NO: 4394 (700454970H1), SEQ ID NO: 5450 (700457060H1), SEQ ID NO: 5719 (700457561H1), SEQ ID NO: 5763 (700457660H1), SEQ ID NO: 4725 (700455669H1), SEQ ID NO: 4003 (700454241H1), SEQ ID NO: 5487 (700457122H1), SEQ ID NO: 5264 (700456711H1), SEQ ID NO: 5850 (700457850H1), SEQ ID NO: 5501 (700457141H1), SEQ ID NO: 4085 (700454396H1), SEQ ID NO: 4091 (700454409H1), SEQ ID NO: 4404 (700454989H1), SEQ ID NO: 5390 (700456948H1), SEQ ID NO: 5751 (700457638H1), SEQ ID NO: 4914 (700456003H1), SEQ ID NO: 6035 (700458234H1), SEQ ID NO: 6078 (700458322H1), SEQ ID NO: 5893 (700457927H1), SEQ ID NO: 5309 (700456784H1), SEQ ID NO: 4539 (700455212H1), SEQ ID NO: 5551 (700457240H1), SEQ ID NO: 5522 (700457174H1), SEQ ID NO: 5486 (700457121H1), SEQ ID NO: 4478 (700455106H1), SEQ ID NO: 4139 (700454474H1), SEQ ID NO: 5938 (700458026H1), SEQ ID NO: 6329 (700458889H1), SEQ ID NO: 5155 (700456455H1), SEQ ID NO: 5883 (700457911H1), SEQ ID NO: 3873 (700453966H1), SEQ ID NO: 4652 (700455543H1), SEQ ID NO: 4425 (700455021H1), SEQ ID NO: 4406 (700454992H1), SEQ ID NO: 6084 (700458339H1), SEQ ID NO: 3644 (700405359H1), SEQ ID NO: 5258 (700456692H1), SEQ ID NO: 4088 (700454405H1), SEQ ID NO: 3976 (700454186H1), SEQ ID NO: 4054 (700454337H1), SEQ ID NO: 4704 (700455637H1), SEQ ID NO: 3707 (700405462H1), SEQ ID NO: 6165 (700458510H1), SEQ ID NO: 6058 (700458285H1), SEQ ID NO: 4275 (700454729H1), SEQ ID NO: 3755 (700453757H1), SEQ ID NO: 5566 (700457256H1), SEQ ID NO: 4545 (700455226H1), SEQ ID NO: 4144 (700454483H1), SEQ ID NO: 5216 (700456614H1), SEQ ID NO: 4490 (700455127H1), SEQ ID NO: 5278 (700456729H1), SEQ ID NO: 5260 (700456695H1), SEQ ID NO: 5157 (700456458H1), SEQ ID NO: 5483 (700457116H1), SEQ ID NO: 4032 (700454303H1), SEQ ID NO: 5709 (700457540H1), SEQ ID NO: 4125 (700454456H1), SEQ ID NO: 4503 (700455152H1), SEQ ID NO: 4567 (700455262H1), SEQ ID NO: 5442 (700457051H1), SEQ ID NO: 5573 (700457266H1), SEQ ID NO: 5531 (700457202H1), SEQ ID NO: 4031 (700454302H1), SEQ ID NO: 5381 (700456920H1), SEQ ID NO: 6257 (700458655H1), SEQ ID NO: 4322 (700454836H1), SEQ ID NO: 6059 (700458286H1), SEQ ID NO: 5090 (700456344H1), SEQ ID NO: 4920 (700456014H1), SEQ ID NO: 4555 (700455240H1), SEQ ID NO: 6046 (700458257H1), SEQ ID NO: 3985 (700454209H1), SEQ ID NO: 3988 (700454213H1), SEQ ID NO: 5163 (700456469H1), SEQ ID NO: 5791 (700457720H1), SEQ ID NO: 4363 (700454917H1), SEQ ID NO: 4474 (700455101H1), SEQ ID NO: 4337 (700454859H1), SEQ ID NO: 4340 (700454864H1), SEQ ID NO: 3786 (700453808H1), SEQ ID NO: 3686 (700405427H1), SEQ ID NO: 5691 (700457504H1), SEQ ID NO: 4059 (700454350H1), SEQ ID NO: 4038 (700454313H1), SEQ ID NO: 4039 (700454314H1), SEQ ID NO: 5147 (700456445H1), SEQ ID NO: 4376 (700454942H1), SEQ ID NO: 3997 (700454229H1), SEQ ID NO: 3892 (700454004H1), SEQ ID NO: 5747 (700457630H1), SEQ ID NO: 4835 (700455873H1), SEQ ID NO: 3941 (700454121H1), SEQ ID NO: 3990 (700454221H1), SEQ ID NO: 5011 (700456186H1), SEQ ID NO: 5411 (700456986H1), SEQ ID NO: 5904 (700457948H1), SEQ ID NO: 5642 (700457392H1), SEQ ID NO: 4098 (700454419H1), SEQ ID NO: 5803 (700457750H1), SEQ ID NO: 4955 (700456069H1), SEQ ID NO: 5251 (700456679H1), SEQ ID NO: 6017 (700458194H1), SEQ ID NO: 4159 (700454516H1), SEQ ID NO: 4514 (700455169H1), SEQ ID NO: 5271 (700456720H1), SEQ ID NO: 4426 (700455022H1), SEQ ID NO: 4409 (700454996H1), SEQ ID NO: 4559 (700455247H1), SEQ ID NO: 5472 (700457088H1), SEQ ID NO: 3638 (700405352H1), SEQ ID NO: 5283 (700456741H1), SEQ ID NO: 5781 (700457692H1), SEQ ID NO: 5628 (700457367H1), SEQ ID NO: 5076 (700456324H1), SEQ ID NO: 5890 (700457924H1), SEQ ID NO: 4299 (700454785H1), SEQ ID NO: 3740 (700453730H1), SEQ ID NO: 5761 (700457656H1), SEQ ID NO: 5765 (700457663H1), SEQ ID NO: 4699 (700455630H1), SEQ ID NO: 4346 (700454880H1), SEQ ID NO: 6038 (700458240H1), SEQ ID NO: 4564 (700455257H1), SEQ ID NO: 5895 (700457929H1), SEQ ID NO: 5646 (700457402H1), SEQ ID NO: 4666 (700455575H1), SEQ ID NO: 4665 (700455574H1), SEQ ID NO: 3931 (700454106H1), SEQ ID NO: 4089 (700454406H1), SEQ ID NO: 3953 (700454139H1), SEQ ID NO: 3932 (700454107H1), SEQ ID NO: 3901 (700454033H1), SEQ ID NO: 3890 (700453995H1), SEQ ID NO: 5558 (700457247H1), SEQ ID NO: 4863 (700455922H1), SEQ ID NO: 3841 (700453912H1), SEQ ID NO: 6215 (700458576H1), SEQ ID NO: 4495 (700455139H1), SEQ ID NO: 5560 (700457250H1), SEQ ID NO: 3989 (700454219H1), SEQ ID NO: 6114 (700458404H1), SEQ ID NO: 5189 (700456528H1), SEQ ID NO: 5722 (700457568H1), SEQ ID NO: 4563 (700455255H1), SEQ ID NO: 6040 (700458242H1), SEQ ID NO: 5547 (700457230H1), SEQ ID NO: 4104 (700454426H1), SEQ ID NO: 4681 (700455604H1), SEQ ID NO: 6322 (700458877H1), SEQ ID NO: 3608 (700405305H1), SEQ ID NO: 4261 (700454705H1), SEQ ID NO: 5308 (700456783H1), SEQ ID NO: 5178 (700456503H1), SEQ ID NO: 3758 (700453762H1), SEQ ID NO: 3756 (700453759H1), SEQ ID NO: 3708 (700405463H1), SEQ ID NO: 5243 (700456663H1), SEQ ID NO: 6202 (700458557H1), SEQ ID NO: 6196 (700458549H1), SEQ ID NO: 5339 (700456844H1), SEQ ID NO: 4568 (700455263H1), SEQ ID NO: 5608 (700457336H1), SEQ ID NO: 4939 (700456049H1), SEQ ID NO: 5546 (700457225H1), SEQ ID NO: 5416 (700457006H1), SEQ ID NO: 5911 (700457961H1), SEQ ID NO: 5007 (700456175H1), SEQ ID NO: 5564 (700457254H1), SEQ ID NO: 5565 (700457255H1), SEQ ID NO: 5848 (700457848H1), SEQ ID NO: 4670 (700455579H1), SEQ ID NO: 5388 (700456942H1), SEQ ID NO: 4820 (700455846H1), SEQ ID NO: 5806 (700457760H1), SEQ ID NO: 5873 (700457888H1), SEQ ID NO: 5955 (700458063H1), SEQ ID NO: 5161 (700456463H1), SEQ ID NO: 4117 (700454445H1), SEQ ID NO: 5475 (700457093H1), SEQ ID NO: 3835 (700453894H1), SEQ ID NO: 3881 (700453979H1), SEQ ID NO: 5289 (700456751H1), SEQ ID NO: 4687 (700455611H1), SEQ ID NO: 5778 (700457688H1), SEQ ID NO: 5663 (700457434H1), SEQ ID NO: 6140 (700458451H1), SEQ ID NO: 4441 (700455046H1), SEQ ID NO: 4824 (700455853H1), SEQ ID NO: 3827 (700453878H1), SEQ ID NO: 4130 (700454464H1), SEQ ID NO: 4110 (700454435H1), SEQ ID NO: 5793 (700457726H1), SEQ ID NO: 5506 (700457147H1), SEQ ID NO: 6164 (700458509H1), SEQ ID NO: 4048 (700454329H1), SEQ ID NO: 5424 (700457020H1), SEQ ID NO: 5420 (700457012H1), SEQ ID NO: 6151 (700458468H1), SEQ ID NO: 4947 (700456059H1), SEQ ID NO: 4352 (700454902H1), SEQ ID NO: 6111 (700458395H1), SEQ ID NO: 6220 (700458587H1), SEQ ID NO: 6234 (700458616H1), SEQ ID NO: 4560 (700455252H1), SEQ ID NO: 4392 (700454967H1), SEQ ID NO: 4881 (700455944H1), SEQ ID NO: 4929 (700456036H1), SEQ ID NO: 5568 (700457258H1), SEQ ID NO: 5570 (700457260H1), SEQ ID NO: 5545 (700457223H1), SEQ ID NO: 4523 (700455179H1), SEQ ID NO: 5750 (700457637H1), SEQ ID NO: 4873 (700455936H1), SEQ ID NO: 6186 (700458537H1), SEQ ID NO: 6198 (700458552H1), SEQ ID NO: 5414 (700456994H1), SEQ ID NO: 5799 (700457740H1), SEQ ID NO: 5669 (700457445H1), SEQ ID NO: 5754 (700457644H1), SEQ ID NO: 4640 (700455518H1), SEQ ID NO: 3785 (700453807H1), SEQ ID NO: 6245 (700458639H1), SEQ ID NO: 3844 (700453919H1), SEQ ID NO: 3682 (700405418H1), SEQ ID NO: 3627 (700405340H1), SEQ ID NO: 5005 (700456169H1), SEQ ID NO: 4948 (700456061H1), SEQ ID NO: 4355 (700454907H1), SEQ ID NO: 4894 (700455964H1), SEQ ID NO: 5485 (700457120H1), SEQ ID NO: 4586 (700455286H1), SEQ ID NO: 4410 (700455001H1), SEQ ID NO: 4375 (700454941H1), SEQ ID NO: 4251 (700454684H1), SEQ ID NO: 6278 (700458802H1), SEQ ID NO: 4880 (700455943H1), SEQ ID NO: 5092 (700456346H1), SEQ ID NO: 4830 (700455863H1), SEQ ID NO: 4827 (700455858H1), SEQ ID NO: 5219 (700456619H1), SEQ ID NO: 5629 (700457371H1), SEQ ID NO: 5404 (700456971H1), SEQ ID NO: 4525 (700455183H1), SEQ ID NO: 4312 (700454820H1), SEQ ID NO: 3860 (700453944H1), SEQ ID NO: 5676 (700457457H1), SEQ ID NO: 5673 (700457451H1), SEQ ID NO: 5915 (700457969H1), SEQ ID NO: 5108 (700456369H1), SEQ ID NO: 4115 (700454443H1), SEQ ID NO: 4518 (700455174H1), SEQ ID NO: 4359 (700454913H1), SEQ ID NO: 5493 (700457130H1), SEQ ID NO: 4057 (700454344H1), SEQ ID NO: 4975 (700456105H1), SEQ ID NO: 3803 (700453835H1), SEQ ID NO: 3799 (700453827H1), SEQ ID NO: 4953 (700456066H1), SEQ ID NO: 5672 (700457449H1), SEQ ID NO: 4290 (700454767H1), SEQ ID NO: 3742 (700453733H1), SEQ ID NO: 6290 (700458825H1), SEQ ID NO: 6248 (700458643H1).

Analogous to the nucleic acid sequences, a CDP may have an amino acid sequence which is naturally occurring, synthetic, or variant. Guidance in determining which amino acid residues may be substituted, inserted, or deleted without abolishing biological or immunological activity may be found using computer programs such as LASERGENE Navigator (DNASTAR, Madison Wis.) which are well known in the art.

Derivation of Nucleic Acid Sequences

In the present embodiment, mRNA was isolated from corn seedling and used to construct the SATMON012 and SATMON029 cDNA libraries. Random cDNA isolates were sequenced in part and analyzed using the homology programs described below. The sequences of the isolates are disclosed in the Sequence Listing. These cdps may contain either a partial or a full length open reading frame, or they may contain all or part of a regulatory element for a particular gene. This variation is attributed to the fact that many genes are several hundred, and sometimes several thousand, bases in length. With current technology, large genes cannot be cloned in their entirety because of vector limitations, incomplete reverse transcription of the first strand, or incomplete replication of the second strand. This is particularly common in libraries generated by random priming, since first strand synthesis may begin anywhere within the transcript. Contiguous, secondary clones containing additional nucleotide sequences may be obtained using a variety of methods known to those of skill in the art.

Secuencing of the cDNAs

Methods for DNA sequencing are well known in the art. Conventional enzymatic methods employ DNA polymerase, Klenow fragment, THERMO SEQUENASE DNA polymerase (Amersham Pharmacia Biotech, Piscataway N.J.), or Taq DNA polymerase (Amersham Pharmacia Biotech) to extend the nucleic acid sequence from an oligonucleotide primer annealed to the DNA template of interest. Methods have been developed for the use of both single-stranded and double-stranded templates. Chain termination reaction products may be electrophoresed on urea-polyacrylamide gels and detected either by autoradiography (for radionucleotide-labeled nucleotides) or by fluorescence (for fluorescent-labeled nucleotides). Recent improvements in mechanized reaction preparation, sequencing, and analysis using the fluorescent detection method have permitted expansion in the number of inserts that may be sequenced per day using machines such as the ABI 377 DNA Sequencer (Perkin Elmer, Norwalk Conn.).

Reading Frame Determinations

The reading frame of the nucleotide sequence may be ascertained by several types of analyses. First, reading frames contained within the coding sequence may be analyzed for the presence of start (ATG, GTG, etc.) and stop codons (TGA, TAA, TAG). Typically, one reading frame will continue throughout the major portion of a cDNA sequence while the other two reading frames tend to contain numerous stop codons. For more difficult cases, algorithms have been created to analyze the occurrence of individual nucleotide bases at each putative codon triplet (Fickett, J. W. (1982) Nucl. Acids Res. 10:5303-5318). Coding sequences for particular organisms (bacteria, plants, and animals) tend to contain certain triplet periodicities, such as a significant preference for pyrimidines in the third codon position. These preferences have been incorporated into widely available software which may be used to determine the coding potential and frame of a given stretch of DNA. Coding preferences and start/stop codon information may be used to determine proper frame with a high degree of certainty which, in turn, permits cloning of the sequence in the correct frame.

The nucleotide sequences of the Sequence Listing have been prepared by current, state-of-the-art, automated methods and, as such, may contain occasional sequencing errors and unidentified nucleotides. Such unidentified nucleotides are designated by an N. The infrequent sequencing errors or N's in the nucleotide sequences of the Sequence Listing do not present a problem to those skilled in the art who wish to practice the invention. Several methods employing standard recombinant techniques, described in Ausubel, F. M. et al. (1997; Short Protocols in Molecular Biology, John Wiley & Sons, New York N.Y.), Sambrook, J. et al. (1989; Molecular Cloning, A Laboratory Manual, Cold Spring Harbor Press, Plainview N.Y.), or periodic updates thereof, may be used to correct errors and complete the missing sequence information. The same techniques used for obtaining a full-length sequence may be used to obtain a complete and accurate nucleotide sequence.

Homology Searches

The nucleic acid sequences of the Sequence Listing were used as query sequences against GenBank, or other databases available to the public, to determine homology to known sequences. Illustrative of computer programs known to those of skill in the art for performing computer-assisted nucleic acid or amino acid homology searches is the program Basic Local Alignment Search Tool or BLAST (Altschul, S. F. (1993) J. Mol. Evol. 36:290-300; Altschul, et al. (1990) J. Mol. Biol. 215:403-410). BLAST produces alignments of both nucleotide and amino acid sequences to determine sequence similarity. BLAST is especially useful in determining exact matches or homology. GenBank databases may be searched for sequences containing regions of homology to a query cdp of the present invention. Other databases (such as SwissProt, BLOCKS, or Pima II) may be searched for regions of amino acid sequence homology corresponding to the deduced CDP.

As described in Karlin (supra), the fundamental unit of BLAST algorithm output is the High-scoring Segment Pair (HSP). An HSP consists of two sequence fragments of arbitrary, but equal lengths, whose alignment is locally maximal and for which the alignment score meets or exceeds a threshold or cutoff score set by the user. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the BLAST program output.

BLAST may be used with any of the cdps of the present invention to search for HSPs between a query sequence and sequences in a reference nucleotide or protein database. The statistical significance of any matches is evaluated, and those matches that satisfy the user-selected threshold of significance are reported.

Homologous sequences, as determined by a BLAST search, may include prokaryotic (bacterial) or eukaryotic (animal, fungal, or plant) sequences. Where a cdp represents only part of a gene, the degree of homology is based only on the partial sequence disclosed in the Sequence Listing. When sequences have sufficiently long regions of agreement or sufficiently high overall agreement, the score of the cdp is considered to be a nearly exact match. Allelic sequences fit this category when they only differ by about three nucleotides per 100. Homologous matches between the cdps provided in the Sequence Listing and the GenBank databases are reported in TABLES 1 and 2.

Corn Seedling-Derived Sequences

The cdps of the present invention may serve to identify, evaluate, alter, or follow the inheritance of desired characteristics associated with growth and development, disease resistance, environmental adaptability, quality, and yield of corn. In particular, cdps are useful as molecular markers for studying inheritance of multigene traits in a plant breeding program.

Hybridization and Genetic Analysis

The cdps, their oligonucleotides, fragments, or complementary sequences, may be used to identify the presence of and/or to determine the degree of similarity between two (or more) nucleic acid sequences. The cdps may be hybridized to naturally occurring or recombinant nucleic acid sequences under appropriately selected temperatures and salt concentrations. Hybridization with a probe based on the nucleic acid sequence of at least one of the cdps allows for the detection of nucleic acid sequences, including genomic sequences, which are identical or closely homologous to the cdps of the Sequence Listing. Probes may be selected from non-conserved or unique regions of the cdps of interest and pretested for their ability to identify or amplify the target nucleic acid sequence using standard protocols. Optimization of the protocol, e.g. increasing stringency to reduce the frequency of false positives or avoiding polyadenylated or other regions predicted to provide secondary structure to reduce false negatives, should provide the desired results. A labeled probe may be used to detect or quantify cDNAs, endogenous corn transcripts, or genes. As will be understood by those of skill in the art, hybridization conditions, probe length and labeling will vary depending upon the intended use. Hybridization conditions, based on the melting temperature (Tm) of the probe and on the salt concentrations under which hybridization and subsequent washes are carried out are well known in the art and are taught in Sambrook (supra) and Ausubel (supra).

A probe for use in Southern or northern hybridization may be a cdp sequence or its complement that is up to several hundred nucleotides long and either single-stranded or double-stranded. Such probes may be hybridized in solution to biological materials such as plasmids, bacterial or yeast artificial chromosomes, cleared plant tissues, etc. or to artificial substrates containing cdps. Microarrays are particularly suitable for identifying the presence and detecting the level of gene expression of multiple desired traits by examining gene expression of selected inbreds and hybrids at various stages of development. An array analogous to a dot or slot blot may be used to arrange and link the cDNA fragments or oligonucleotides to the surface of a substrate using one or more of the following: mechanical (vacuum), chemical, thermal, or UV bonding procedures. Such an array may contain any number of cdps and may be produced by hand or by using available devices, materials, and machines.

Probes may be labeled by either PCR or enzymatic techniques using a variety of reporter molecules. Commercial kits are available for radioactive labeling and probe cleanup from Amersham Pharmacia Biotech, for alkaline phosphatase labeling from Life Technologies (Rockville Md.), for chemiluminescent labeling from Lumigen (Southfield Mich.), etc. Alternatively, cdps may be cloned into commercially available vectors for the production of RNA probes. Such probes may be transcribed in the presence of at least one labeled nucleotide (e.g. [α⁻³²P] CTP, Amersham Pharmacia Biotech).

Genetic maps, based upon molecular markers (restriction fragment length polymorphisms, RFLPs) are being assembled for several grains including rice, corn, barley, and wheat. These maps have improved understanding and manipulation of both single and multigene traits. Even when the genes involved are unknown, the ability to show the presence of the associated marker and the desired characteristics in inbred or hybrid corn plants and to follow segregation in a breeding program make the marker valuable as a diagnostic. Moreover, continuous variation within a segregating family may often be resolved into a handful of major gene effects associated with molecular markers. As genetic maps merge with physical maps, it becomes possible to walk along the chromosome and clone virtually any gene. Hybridization and newer technologies such as random amplified polymorphic DNA (RAPD) analysis, microsatellites and amplified fragment length polymorphisms (AFLP) make it easier to isolate the actual genes which interact and are responsible for a desired trait.

Diagnostic Uses

Diagnostic assays known to those of skill in the art may be used to detect or confirm conditions or diseases associated with abnormal levels of cdp expression. Labeled probes developed from the nucleotide sequence encoding a cdp are added to a plant sample under amplifying or hybridizing conditions. The complex between the naturally occurring sequence and the labeled probe is quantified and compared with a standard for that cell or tissue. If cdp expression varies significantly from the standard, the assay indicates the presence of the condition or disease. Qualitative or quantitative diagnostic methods may include northern, dot blot, or other membrane or dip-stick based technologies or multiple-sample format technologies such as PCR, ELISA-like, pin, or chip-based assays. The determination of whether cdp expression in a sample varies significantly from a standard is determined by methods of statistical analyses well known to those of skill in the art.

Accordingly, the invention provides a method for assessing disease resistance or other conditions using a panel of probes. A candidate probe is identified from CDPs which are specific to corn tissue and have not been observed in GenBank or other Incyte-sequenced cDNA libraries. The usefulness of the probe may be tested by quantifying its hybridization across tissues which are normal versus diseased. Once an increase (or decrease) in expression level is related to a trait such as fungal resistance, the probe can be used to monitor ability of a particular inbred or hybrid corn line to withstand fungal infection.

Transcript Imaging

Another embodiment relates to development of diagnostic or treatment methods based on specific imaging of the cdps of the present invention. The profile of nucleic acid sequences which reflect gene transcription activity in a particular cell type, tissue, or plant at a particular time, is defined as a “transcript image”. Such profiles are generated by naming, matching, and counting all copies of related clones and arranging them in order of abundance.

Clones may also be arranged in clusters in descending order of abundance. The minimum number of clones necessary to constitute a cluster, as illustrated at the bottom of TABLE 2, is two. All clones in TABLE 2 are seedling specific although individual clusters may consist of either unique cdps or cdps that are homologous to known sequences. An alternative presentation of this data might involve a spreadsheet which contains cluster abundance data as well as some descriptive information for the homologous clones.

Subtractions, or subsetting, among transcript images may be used to discern various differences in gene expression and cellular activities. For example, subsetting may be used with the PHYTOSEQ database (Incyte Pharmaceuticals, Palo Alto Calif.) to show differences between: a) organs of two different developmental stages; b) two different organs, such as leaves and roots; c) organs from two different species; or d) normal and diseased or stressed plant tissues.

Large numbers of mRNA transcripts, as represented by their respective CDNA clones, may be compared using computational methods rather than analogous laboratory methods, such as northern blot analysis. For example, electronic subtraction between any two transcript images parallels hybrid subtraction between any two cDNA libraries (cf. Sambrook, supra). The information produced by the subtraction of transcript images between different libraries may be used to select single or multiple cdps which may be used to predict yield.

A cdp identified through transcript imaging, or other means, may also be used to clone regulatory elements for use in transformation vectors. Expression may be quantified using amplification or microarray technologies which are well known in the art.

Complementary Strand

The cdp, or any part thereof, may be used as a tool in technologies for altering gene expression. To inhibit in vivo or in vitro cdp transcription, a PNA (Nielsen, P. E. et al. (1993) Anticancer Drug Des. 8:53-63) or an oligonucleotide based on the sequence of a cdp is designed using OLIGO 4.06 software (National Biosciences, Plymouth Minn.) or LASERGENE Navigator (DNASTAR). Alternatively, a fragment of a cdp is cloned into an expression vector which is transformed into a host cell to express the complementary strand. An analogous molecule may be designed to inhibit promoter binding in the upstream nontranslated leader or at various sites along the 5′ coding region of the cdp. Alternatively, complementary molecules may be designed to inhibit translation of an mRNA by preparing an oligomer or fragment which binds to the transcript preventing its association with the ribosomal machinery.

Complementary molecules may also be designed to disrupt genomic sequences (such as enhancers, introns) preventing the normal activity of these regulatory elements. Similarly, complementary strands may be used in a process known as “triple helix” base pairing to inhibit replication. These molecules compromise the ability of the double helix to open and bind to polymerases and transcription factors necessary for replication.

Stable transformation of appropriate dividing cells with an expression vector encoding the complement of a cdp may produce a transgenic cell line, tissue, or organism. Those cells which assimilate, replicate, and express the nucleic acid sequence in sufficient quantities may compromise or entirely eliminate the natural activity of the cdp. Frequently, the function of a cdp may be ascertained by observing lethality, loss of physiological activity, changes in morphology, etc. at the cellular, tissue, organ, or organismal level.

Expression

The cdps may be used in recombinant vectors to express a polypeptide. It may be advantageous to design nucleic acid sequences possessing the GC ratio of codons preferred by a particular prokaryotic or eukaryotic host (Murray, E. et al. (1989) Nuc. Acids Res. 17:477-508). In addition, 3′ terminators, such as bacterial nopalene synthase or octapine synthase, may be modified, or substituted into vectors, to produce transcripts having more desirable properties, such as a longer half-life, than transcripts produced from the naturally occurring sequence (Sullivan, M. L. and Green, P. J. (1993) Plant Mol. Biol. 23:1091-1104; Silva, E. M. et al. (1987) J. Cell Biol. 105:245). The cdps may also be altered by site-directed mutagenesis to insert new restriction sites and to modify the peptide by glycosylation, phosphorylation, acetylation, etc.

The cdp may be ligated to a heterologous sequence to create a chimeric or fusion protein. For ease of purification, it may be useful to produce a fusion protein that is recognized by a commercially available antibody. In addition, the sequence may be engineered to introduce a cleavage site between the peptide of interest and the heterologous protein sequence, so that the peptide may be cleaved from the heterologous moiety and purified.

Alternately, the peptide may be synthesized, whole or in part, using chemical methods well known in the art. For example, peptides may be synthesized using various solid-phase techniques (Roberge, J. Y. et al. (1995) Science 269:202-204) or an Peptide Synthesizer Model 431A (Perkin Elmer) using instructions provided by the manufacturer. Once synthesized, the peptide may be purified by preparative high performance liquid chromatography, and composition confirmed by amino acid sequencing (Ausubel (supra) p. 10.82f).

Expression Systems

For protein expression, the nucleic acid sequence may be inserted into an expression vector which contains the necessary elements for appropriate transcription and translation. Methods which are well known to those skilled in the art may be used to construct such vectors. These methods include in vitro recombinant DNA techniques, synthetic techniques, in vivo recombination, or genetic recombination. Such techniques are described in Sambrook (supra) and Ausubel (supra). One of the advantages of producing the CDPs by recombinant DNA technology is the ability to obtain highly-enriched sources of the polypeptides that simplify purification procedures.

The cdps may be engineered into a variety of expression vectors and host cells. These include, but are not limited to, microorganisms such as bacteria transformed with recombinant bacteriophage, plasmid or cosmid DNA expression vectors; yeast transformed with yeast expression vectors; insect cell systems infected with virus expression vectors (e.g. baculovirus); plant cells transfected with expression vectors containing viral, bacterial, or eukaryotic elements (e.g. cauliflower mosaic virus, CaMV; Ti or pBR322 plasmids; and cell or seedling-specific, constitutive or inducible, monocot or corn elements).

The regulatory elements of vectors vary in their strength and specificities and are those nontranslated regions such as enhancers, promoters, introns, and 3′ untranslated regions which interact with host proteins to carry out transcription and translation. Depending on the vector and host, any number of suitable transcription and translation elements may be used. For example, promoters or enhancers derived from the genomes of plant cells (e.g. heat shock, RUBISCO; and storage protein genes) or from plant viruses (e.g. viral promoters or leader sequences) may be cloned into the vector containing an appropriate selectable marker. In fact, the cdps of this invention may be used to clone upstream, tissue-specific or inducible regulatory elements for purposes of engineering and expressing heterologous genes in corn.

In a bacterial system, an expression vector may be selected to direct a high level expression of a fusion protein. Commercial vectors include, but are not limited to, the multifunctional E. coli cloning and expression vectors, PBLUESCRIPT (Stratagene, La Jolla Calif.) and PSPORT (Life Technologies). Using either of these vectors, the nucleic acid sequence may be ligated into the vector in-frame with sequences for the amino-terminal Met and the subsequent 7 residues of β-galactosidase so that a chimeric protein is produced. PGEX vectors (Amersham Pharmacia Biotech) may also be used to express peptides by ligating the nucleic acid sequence to glutathione S-transferase (GST). In general, such fusion proteins are soluble and may easily be purified from lysed cells by adsorption to glutathione-agarose beads followed by elution in the presence of free glutathione. Proteins made in such systems are designed to include heparin, thrombin, or factor X_{A} protease cleavage sites so that the peptide of interest may be released from the GST moiety at will.

In plants, the expression of nucleic acid sequences may be driven by any of a number of promoters. Viral promoters such as the 35S and 19S promoters of CaMV may be used alone or in combination with the omega leader sequence from TMV (Takamatsu, N. et al. (1987) EMBO J. 6:307-311). Alternatively, plant promoters such as the small subunit of RUBISCO (Coruzzi, G. et al. (1984) EMBO J. 3:1671-1680; Broglie, R. et al. (1984) Science 224:838-843); or heat shock promoters (Winter, J. and Sinibaldi, R. M. (1991) Results Probl. Cell Differ. 17:85-105) may be used. Preferably, the cdps of the invention may be used to identify clones containing full length genes by hybridization or to clone full length genes or regulatory elements for use in expression vectors by PCR.

X-Ray Crystallography

Expression of the recombinant CDP in sufficient amounts may make analytical studies such as X-ray crystallography possible. In the alternative, knowledge of the amino acid sequence deduced from the nucleic acid sequence may provide guidance to those employing computer modeling techniques in place of or in addition to X-ray crystallography.

Antibodies

Anti-CDP antibodies may be produced to use in assays of protein expression. Such antibodies include, but are not limited to, polyclonal, monoclonal, chimeric, single chain, Fab fragments and fragments produced by a Fab expression library. Neutralizing antibodies, i.e., those which inhibit dimer formation, are especially useful for diagnostics.

The amino acid sequence encoded by the cdps of the Sequence Listing may be analyzed by appropriate software (e.g. LASERGENE Navigator, DNASTAR) to determine regions of high immunogenicity. The optimal sequences for immunization are selected from the C-terminus, the N-terminus, and those intervening, hydrophilic regions of the peptide which are likely to be exposed to the external environment when the peptide is in its natural conformation. Analysis used to select appropriate epitopes is also described by Ausubel (supra, unit 11-7). Peptides used for antibody induction do not need to have biological activity; however, they must be antigenic. Peptides used to induce specific antibodies may have an amino acid sequence consisting of at least five amino acids, and preferably at least 10 amino acids. An oligopeptide should mimic an antigenic portion of the natural peptide and may be fused with another protein such as KLH (Sigma-Aldrich) for antibody production. An oligopeptide or peptide encompassing an antigenic region may be expressed from the nucleic acid sequence, synthesized, or purified from corn.

Procedures well known in the art may be used for the production of antibodies. Various hosts including mice, goats, and rabbits, may be immunized by injection with a peptide or oligopeptide. Depending on the host species, various adjuvants may be used to increase immunological response.

In one procedure, oligopeptides about 15 residues in length may be synthesized using a Peptide Synthesizer Model 431A (Perkin Elmer) using Fmoc-chemistry and coupled to KLH (Sigma-Aldrich) by reaction with M-maleimidobenzoyl-N-hydroxysuccinimide ester (Ausubel, supra). If necessary, a cysteine may be introduced at the N-terminus of the oligopeptide to permit coupling to KLH. Rabbits are immunized with the oligopeptide-KLH complex in complete Freund's adjuvant. The resulting antisera are tested for antipeptide activity by binding the peptide to plastic, blocking with 1% BSA, reacting with rabbit antisera, washing, and reacting with radioiodinated goat anti-rabbit IgG.

In another procedure, the peptide, in quantities up to 75 mg, may be used to immunize mice or rabbits. About 100 μg are used to immunize a mouse, while up to 1 mg is used to immunize a rabbit. Subsequently, the peptide is radioiodinated and used to screen the B-lymphocyte cells from the immunized animal for production of hybridomas using standard techniques. About 20 mg of protein are sufficient for labeling and screening several thousand clones.

Hybridomas may also be prepared and screened using standard techniques. Hybridomas of interest are detected by screening with radioiodinated peptide to identify those fusions producing peptide-specific monoclonal antibody. In a typical protocol, wells of microtiter plates are coated with affinity-purified, specific rabbit-anti-mouse (or suitable anti-species IgG) antibodies at 10 mg/ml. The coated wells are blocked with 1% BSA and washed and exposed to supernatants from hybridomas. After incubation, the wells are exposed to radiolabeled peptide at 1 mg/ml. Clones producing antibodies bind a quantity of labeled peptide that is detectable above background.

Such clones are expanded and subjected to 2 cycles of cloning at 1 cell/3 wells. Cloned hybridomas are injected into pristane-treated mice to produce ascites, and monoclonal antibody is purified from the ascitic fluid by affinity chromatography on Protein A (Amersham Pharmacia Biotech). Monoclonal antibodies with affinities of at least 10⁸ M⁻¹, preferably 10⁹ M⁻¹ to 10^{10 M}⁻¹ or greater, are made by standard procedures as described in Harlow (1988; Antibodies: A Laboratory Manual, Cold Spring Harbor Laboratory, Cold Spring Harbor N.Y.) and Goding (1986; Monoclonal Antibodies: Principles and Practice, Academic Press, New York N.Y.).

Antibody fragments which contain specific binding sites for an epitope may also be generated. For example, such fragments include, but are not limited to, the F(ab′)2 fragments which may be produced by pepsin digestion of the antibody molecule and the Fab fragments which may be generated by reducing the disulfide bridges of the F(ab′)2 fragments. Alternatively, Fab expression libraries may be constructed to allow rapid and easy identification of monoclonal Fab fragments with the desired specificity (Huse, W. D. et al. (1989) Science 256:1275-1281).

Assays Using Antibodies

Anti-CDP antibodies may be used to determine the amount of CDP found in a particular cell of a corn inbred line or hybrid under various environmental or disease conditions. Assays for such peptides include methods utilizing the antibody and a label to detect expression in plant extracts, cells, or tissues. The peptides and antibodies of the invention may be used with or without modification. Frequently, the peptides and antibodies will be labeled by joining them, either covalently or noncovalently, with a reporter molecule.

Protocols for detecting and measuring protein expression using either polyclonal or monoclonal antibodies, are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes on a peptide is preferred, but a competitive binding assay may be employed. Such immunoassays typically involve the formation of complexes between the CDP and its specific antibody and the measurement of such complexes. These and other assays are described, among other places, in Hampton, R. et al. (1990, Serological Methods, a Laboratory Manual, APS Press, St Paul Minn.) and Maddox, D. E. et al. (1983, J. Exp. Med. 158:1211-1226).

Screening for Useful Compounds

The cdps or CDPs are particularly useful for screening libraries of molecules or test compounds for identification of those molecules which bind specifically to them. For example, a cdp or fragment thereof may be combined with a plurality of natural, synthetic, or inorganic molecules to screen for molecules that bind to them and function as transcription factors, enhancers, or other cellular elements which contribute to gene expression. Similarly, a CDP or portion thereof may be combined with a plurality of molecules to screen for molecules which bind to them. Molecules identified by screening with cdps or CDPs preferably affect growth and development, disease resistance, environmental adaptability, quality, or yield.

Technologies with multiple-sample format, ELISA-like, capillary, or chip-based assays, are well known in the art and allow large-scale screening. These methods use complex formation, quantification, and comparison with a standard to detect molecules which specifically bind the cdps or CDPs. In the assay, the cdp or CDP may be free in solution, affixed to a substrate, borne on a cell surface, or located intracellularly. For example, prokaryotic host cells which are stably transformed with recombinant nucleic acids that express and position a CDP on the cell surface can be used to screen for molecules which specifically bind the CDP. Viable or fixed cells are screened against a plurality of test compounds and the specificity of binding or formation of complexes between an expressed CDP and the test compound is measured.

Transformation

Bacterial and plant transformation systems are well known in the art. Expression vectors may be introduced into suitable E. coli cells by electroporation, heat shock or other means as described in Ausubel (supra) for the purpose of expressing a plant protein. Expression vectors may also be introduced into plant cells by direct transfer of DNA or pathogen-mediated transfection. For reviews, see McGraw Hill Yearbook of Science and Technology (1992; McGraw Hill New York N.Y., pp 191-196); or Weissbach, A. and Weissbach, H. (1988; Methods Enzymol. 118:421-463). Direct transfer of DNA into plant protoplasts or cells is one approach for transforming plants genetically. DNA uptake by protoplasts may be promoted chemically with polyethylene glycol or electrically with a high-voltage pulse. Both of these methods depend upon a cell culture system to recover plants from a single transformed cell (Rhodes, C. A. et al. (1988) Biotechnology 6:56-60; Morocz, S. (1991) Theor. Appl. Genet. 80:721-726). Regeneration of transformed, fertile plants has been demonstrated in several cereals including rice (Zhang, H. M. (1988) Plant Cell Rep. 7:379-384).

Electroporation, lipofection, microinjection, particle bombardment, vacuum infiltration, and electrotransformation may be used to transform corn cells and embryos. Gordon-Kamm, W. J. et al. (1992; Plant Mol. Biol. 18:201-210) used particle bombardment to transform embryogenic, suspension culture cells; Murry, L. E. et al. (In: Bajaj, Y. P. S. (1994) Biotechnology in Agriculture and Forestry 25:252-261) used continuous, low voltage electric current to transform embryos; and Rhodes, C.A. et al. (1995; Methods Mol. Biol. 55:121-131) describe the electroporation of embryos. Stable transformation requires the use of an expression vector which contains an appropriate origin of replication and gene cassettes containing viral or plant expression elements, a selectable or visible marker, and a gene of interest. Following the introduction of the vector, cells may be allowed to grow for 1-2 days in an enriched media. If the vector contains a selectable marker, the cells are switched to selective media. The selectable marker confers resistance to selective agents and allows growth and recovery of those cells which successfully express the introduced sequences.

Any number of selection systems may be used to recover transformed cell lines. Antimetabolite, antibiotic or herbicide resistance may be used as the basis for selection using genes such as dhfr, which confers resistance to methotrexate (Wigler, M. et al. (1980) Proc. Natl. Acad. Sci. 77:3567-3570); npt, which confers resistance to the aminoglycosides, neomycin and G-418 (Colbere-Garapin, F. et al. (1981) J. Mol. Biol. 150:1-14); and als or pat, which confer resistance to chlorsulfuron and phosphinotricin acetyltransferase, respectively (McGraw Hill Yearbook of Science and Technology, supra). Recently, the use of visible markers has gained popularity with such markers as anthocyanins, β glucuronidase and its substrate, GUS, luciferase and its substrate, luciferin, and green fluorescent protein, GFP, being widely used not only to identify transformants, but also to quantify the amount of transient or stable protein expression attributable to a specific vector system (Rhodes (1995) supra; Haseloff, J. and Amos, B. (1995) Trends Genet. 11:328-329). Plant expression vectors contain 5′ promoters, enhancers and 3′ terminators that will function in the plant cell.

Identification of Transformants

Although the presence/absence of marker gene expression suggests that the gene of interest is also present, its expression should be confirmed. For example, if the sequence is inserted within a marker gene sequence, cells containing the recombinant sequence may be identified by the absence of marker gene function. Alternatively, a marker gene may be placed in tandem with the nucleic acid sequence under the control of a single promoter. Expression of the marker gene in response to induction or selection may indicate the presence and expression of the tandem sequence as well.

Alternatively, host cells which contain the introduced nucleic acid sequence may be identified by a variety of procedures known to those of skill in the art. These procedures include, but are not limited to, DNA-DNA or DNA-RNA hybridization and protein bioassay or immunoassay techniques which include membrane, solution, gel, or chip based technologies for the detection and/or quantification of the nucleic or amino acids and any of the molecules to which they bind.

The presence of the nucleic acid sequence may be detected by DNA-DNA or DNA-RNA hybridization or amplification using probes comprising all or a portion of a nucleic acid sequence. Nucleic acid amplification based assays involve the use of oligonucleotides or oligomers based on the nucleic acid sequence to detect transformants containing the introduced DNA.

A wide variety of labels and conjugation techniques are known by those skilled in the art and may be used in various nucleic acid and amino acid assays. Means for producing labeled hybridization or PCR probes for detecting related sequences include oligolabeling, nick translation, end-labeling or PCR amplification and are well known in the art. Alternatively, the nucleic acid sequence may be cloned into a vector for the production of an mRNA probe. Such vectors are known in the art, are commercially available, and may be used to synthesize RNA probes in vitro by addition of an appropriate RNA polymerase such as T7, T3 or SP6 and labeled nucleotides. A number of companies (e.g., Amersham Pharmacia Biotech and Life Technologies) supply commercial kits, reporter molecules, and protocols for these procedures.

### EXAMPLES

### Isolation, Sequence Analysis and Use of Corn Sequences

I Growth Conditions

The corn cDNA libraries, SATMON012 and SATMON029 were constructed from corn tissues grown and prepared as follows. SATMON012 seed was planted on moist filter paper in a covered tray and kept in the dark for one day at which time germination had begun. The trays were moved to the bench top with 27° C./21° C., 15 hr day/9 hr night cycles for two days. At this time the coleorhiza had pushed through the pericarp, and the radicle had just pierced the coleorhiza and was barely visible. The coleoptile had just emerged from the pericarp. The germinating seeds were harvested, frozen immediately in liquid nitrogen, and stored at −80° C.

SATMON029 seed was planted on moist filter paper in a covered tray and kept in the dark for four days with cycles of 15 hours at 27° C. and nine hours at 21° C. After four days in the dark, all seedlings were etiolated. The radicle had penetrated the coleorhiza and was 4 - 5 cm long; and lateral roots were present. The coleoptile had pushed through the pericarp and was 4 - 5 cm long. The tissue was harvested, frozen immediately in liquid nitrogen, crushed, and stored at −80° C.

II CDNA Library Construction

The frozen tissue from SATMON012 and SATMON029 respectively was homogenized, and total RNA was extracted with TRIZOL reagent (Life Technologies). Polyadenylated RNA was isolated from the total RNA using a magnetic Dynabeads MRNA purification kit (Dynal Inc, Lake Success N.Y.). The mRNA was handled according to the recommended protocols in the SuperScript Plasmid System for CDNA synthesis and plasmid cloning (Life Technologies). The cDNAs for SATMON012 were fractionated on a SEPHAROSE CL-4B column (Amersham Pharmacia Biotech), and those cDNAs exceeding 400 bp were ligated into the NotI and SalI sites of the PSPORT1 plasmid (Life Technologies). The plasmid PSPORT1 was subsequently transformed into DH10B competent cells (Life Technologies). The cDNAs for SATMON029 were fractionated on a SEPHAROSE CL-4B column (Amersham Pharmacia Biotech), and those cDNAs exceeding 400 bp were ligated into PINCY 1 (Incyte Pharmaceuticals). The plasmid pINCY 1 was subsequently transformed into DH10B competent cells (Life Technologies).

III Isolation and Sequencing of CDNA Clones

The plasmid DNA was released from the cells and purified using the R. E. A. L. Prep 96 plasmid kit (Qiagen, Valencia Calif.). This kit enabled the simultaneous purification of 96 samples in a 96-well block using multi-channel reagent dispensers. The recommended protocol was employed except for the following changes: 1) the bacteria were cultured in 1 ml of sterile Terrific Broth (Life Technologies) with carbenicillin at 25 mg/l and glycerol at 0.4%; 2) after inoculation, the cultures were incubated for 19 hours and at the end of incubation, the cells were lysed with 0.3 ml of lysis buffer; and 3) following isopropanol precipitation, the plasmid DNA pellet was resuspended in 0.1 ml of distilled water. After the last step in the protocol, samples were transferred to a 96-well block for storage at 4° C.

In the alternative, DNA was isolated using the following protocols. Single bacterial colonies were transferred into individual wells of the 384-well plates (Genetix, Christchurch UK) using sterile toothpicks. The wells contained 1 ml of sterile Terrific Broth (Life Technologies) with 25 mg/l carbenicillin and 0.4% glycerol (v/v). The plates were covered and placed in a THERMODYNE incubator (Thermodyne Corp, Newtown Square Pa.) at 37° C. for 8-10 hours prior to use. Plasmid DNA was released from the cells and amplified using direct link PCR (Rao, V.B. (1994) Anal. Biochem. 216:1-14) as follows. The direct link PCR solution included 30 ml of NUCLEIX PLUS PCR nucleotide mix (Amersham Pharmacia Biotech) and 300 μl of Taq DNA polymerase (Amersham Pharmacia Biotech) with or without 12 μl Pfu DNA polymerase (Stratagene). Five microliters of the PCR solution were added to each of the 384 wells using the Hydra-96 microdispenser (Hamilton, Reno Nev.); plates were centrifuged at 1000 rpm for 20 seconds and refrigerated until use. A 384 pin tool (V&P Scientific, San Diego Calif.) was used to transfer bacterial cells from the incubation plate into the plate containing the PCR solution where the component 0.1% Tween 20(polyoxyethylene(20)sorbitan monolaurate) caused the cells to undergo lysis and release the plasmid DNA. After lysis, the plates were centrifuged up to 500 rpm, covered with a cycle sealer, and cycled using a 384-well Peltier Thermal Cycler (PCT-200; MJ Research, Watertown Mass.) using the program dPCR30 with the following parameters: Step 1) 95° C., 1 minute; Step 2) 94° C., 30 seconds; Step 3) 55° C., 30 seconds; Step 4) 72° C., 2 minutes; Step 5) steps 2, 3, and 4 repeated 29 times; Step 6) 72° C., 10 minutes; and Step 7) storage at 4° C.

The concentration of DNA in each well was determined by dispensing 100 μl PICOGREEN quantitation reagent (Molecular Probes, Eugene Oreg.) (0.25% reagent dissolved in 10 mM TrisHCl, pH 7.5, 1 mM ethylenediamine tetraacetic acid (EDTA) (1×TE, v/v), and 0.5 μl of undiluted PCR product into each well of an opaque fluorimeter plate (Corning Costar, Acton Mass.) and allowing the DNA to bind to the reagent. The plate was scanned in a Fluoroskan II (Labsystems Oy, Helsinki, Finland) to measure the fluorescence of the sample and to quantify the concentration of DNA.

The cDNAs were prepared and sequenced by the method of Sanger, F. and A. R. Coulson (1975; J. Mol. Biol. 94:441-448), using either a MICROLAB 2200 system (Hamilton) or a HYDRA microdispenser (Robbins Scientific, Sunnyvale Calif.) in combination with PTC-200 cyclers (MJ Research) and ABI PRISM 377 DNA sequencing systems (Perkin Elmer). Most of the isolates were sequenced according to standard PE protocols and kits (Cat. #79345, 79339, 79340, 79357, 79355). The solution volumes were used at 0.25x−1.0x concentrations. In the alternative, cDNAs may have been sequenced using solutions and dyes from Amersham Pharmacia Biotech.

IV Homology Searching of cDNA Clones and Their Deduced Proteins

After the reading frame was determined, the nucleotide sequences of the Sequence Listing or their deduced amino acid sequences were queried against databases such as GenBank, SwissProt, BLOCKS, and Pima II. These databases which contain annotated sequences were searched for regions of homology (similarity) using BLAST (Altschul (1993) supra; Altschul (1990) supra).

BLAST produced alignments of both nucleic and amino acid sequences to determine sequence similarity. Because of the local nature of the alignments, BLAST was especially useful in determining exact matches or in identifying homologs of viral, prokaryotic, or eukaryotic origin. Other algorithms such as the one described in Smith, T.F. (1992; Protein Engineering 5:35-51) could have been used to deal with primary sequence patterns and secondary structure gap penalties. The sequences disclosed in this application have lengths of at least 49 nucleotides, and no more than 12% uncalled bases (where N is recorded rather than A, C, G, or T).

SATMON012 and SATMON029 nucleotide sequences were searched, as described in Karlin (supra), against the GenBank plant (pln) and eukaryote (eukp) databases, and deduced amino acid sequences from the same clones were then searched against GenBank functional protein databases (allp). The relevant database for a particular match was reported in column 5 of TABLE 1. In TABLE 1 column 3, the product score is calculated as follows: the % nucleotide or amino acid identity [between the query and reference sequences] in BLAST is multiplied by the % maximum possible BLAST score [based on the lengths of query and reference sequences] and then divided by 100. In an analogy to the hybridization procedures used in the laboratory, the electronic stringency for an exact match was set at 70, and the conservative lower limit for an exact match was set at approximately 40 (with 1-2% error due to uncalled bases). Column 4 provides log-likelihood where the value=log (probability+threshold) and the threshold for exact matches was set at 10⁻²⁵ for nucleotides and 10⁻¹⁴ for peptides. Column 6 contains a GenBank description of the protein; some of the GenBank descriptions were standardized with respect to abbreviations and spelling.

V Gene Transcript Analysis

The abundance sort program of the invention described in U.S. Pat. No. 5,840,484 entitled “Comparative Gene Transcript Analysis”, incorporated herein by reference, tabulates and sorts by frequency the mRNA transcripts corresponding to each gene identified in a database. The process for obtaining this data set, the profile of corn seedling gene activity or transcript image, is referred to as “gene transcript analysis”.

A transcript analysis summarizes the presence and abundance of exact, unique, and homologous transcripts which are seedling specific. A transcript image may be assembled using TABLE 1, TABLE 2, and the Sequence Listing. Such a collection of sequences is used to characterize minimally active, active, or highly active cdps. Comparisons among normal, diseased, or immature seedling are used to identify those sequences of particular use in predicting yield or in recovering regulatory elements to be used in vectors for genetic engineering. The entire set, or a selected subset, of seedling-specific, unique, or homologous cDNAs may be useful in membrane-based or PCR-based diagnostic technologies.

VI Library Comparisons, Subsetting

LIFESEQ database (Incyte Pharmaceuticals) software is used to compare sets of transcript images for PHYTOSEQ database (Incyte Pharmaceuticals) CDNA libraries. The cdps are filtered by selecting desired values for relative abundance, stringency, and/or product score (described infra). For any particular library, only the subset of cdps that meet the selected values is included in the comparison. Additional filters, such as those to exclude common genes, such as ribosomal proteins, elongation factor, etc. may be used in the search operation. The subsetting of thousands of corn sequences from the transcript images of callus, ear, embryo, endosperm, leaf, meristem, root, seed, seedling, stem, and tassel libraries is used to identify cdps which are of interest.

VII. Extension of CDNA Sequences

The nucleic acid sequence was extended using an Incyte cDNA clone and oligonucleotide primers. One primer was synthesized to initiate 5′ extension of the known fragment, and the other, to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected plant cDNA libraries were used to extend the sequence. If more than one extension was necessary or desired, additional or nested sets of primers were designed. Preferred libraries are ones that have been size-selected to include larger cDNAs. Also, random primed libraries are preferred because they will contain more sequences with the 5′ and upstream regions of genes. A randomly primed library may be particularly useful if an oligo d(T) library does not yield a full-length cDNA. Genomic libraries are useful for extension 5′ of the promoter binding region in obtaining regulatory elements.

High fidelity amplification was obtained by PCR using methods well known in the art. PCR was performed in 96-well plates using the PTC-200 cycler (MJ Research). The reaction mix contained DNA template, 200 nmol of each primer, reaction buffer containing Mg²⁺, (NH₄)₂SO₄, and β-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% v/v; Molecular Probes) dissolved in 1×TE and 0.5 μl of undiluted PCR product into each well of an opaque fluorimeter plate (Corning Costar, Acton Mass.) and allowing the DNA to bind to the reagent. The plate was scanned in a Fluoroskan II (Labsystems Oy) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended nucleotides were desalted and concentrated, transferred to 384-well plates, digested with CvijI cholera virus endonuclease (Molecular Biology Research, Madison Wis.), and sonicated or sheared prior to religation into pUC18 vector (Amersham Pharmacia Biotech). For shotgun sequencing, the digested nucleotides were separated on low concentration (0.6 to 0.8%) agarose gels, fragments were excised, and agar digested with AGARACE enzyme (Promega, Madison Wis.). Extended clones were religated using T4 DNA ligase (New England Biolabs, Beverly Mass.) into pUC18 vector (Amersham Pharmacia Biotech), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2×carbenicillin liquid media.

The cells were lysed, and DNA was amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified using PICOGREEN reagent (Molecular Probes) as described above. Samples with low DNA recoveries were reamplified using the same conditions described above. Samples were diluted with 20% dimethylsulfoxide (DMSO) (1:2, v/v), and sequenced using DYENAMIC energy transfer sequencing primers and the DYENAMIC DIRECT cycle sequencing kit (Amersham Pharmacia Biotech) or the ABI PRISM BIGDYE Terminator cycle sequencing kit (Perkin-Elmer).

VIII Labeling of Probes and Hybridization Analyses

Blotting

Target nucleic acids are isolated from a biological source and applied to a solid matrix (a blot) suitable for standard nucleic acid hybridization protocols by one of the following methods. A mixture of target nucleic acids, a restriction digest of genomic DNA, is fractionated by electrophoresis through an 0.7% agarose gel in 1× TAE (40 mM Tris acetate, −pH 8.5, 2 mM EDTA) running buffer and transferred to a nylon membrane by capillary transfer using 20× saline sodium citrate (SSC). Alternatively, the target nucleic acids are individually ligated to a vector and inserted into bacterial host cells to form a library. Target nucleic acids are then arranged on a blot by one of the following methods. In the first method, bacterial cells containing individual clones are robotically picked and arranged on a nylon membrane. The membrane is placed on bacterial growth medium, LB agar containing carbenicillin, and incubated at 37° C. for 16 hours. Bacterial colonies are denatured, neutralized, and digested with proteinase K. Nylon membranes are exposed to UV irradiation in a STRATALINKER UV-crosslinker (Stratagene) to cross-link DNA to the membrane.

In the second method, target nucleic acids are amplified from bacterial cells by thirty cycles of PCR using primers complementary to vector sequences flanking the insert. Amplified target nucleic acids are purified using SEPHACRYL-400 (Amersham Pharmacia Biotech). Purified target nucleic acids are robotically arrayed onto a glass microscope slide (Corning Science Products, Corning N.Y.). The slide was previously coated with 0.05% aminopropyl silane (Sigma-Aldrich, St. Louis Mo.) and cured at 110° C. The arrayed glass slide (microarray) is exposed to UV irradiation in a STRATALINKER UV-crosslinker (Stratagene).

Probe Preparation

cDNA probes are made from mRNA templates. Five micrograms of mRNA is mixed with 1 μg random primer (Life Technologies), incubated at 70° C. for 10 minutes, and lyophilized. The lyophilized sample is resuspended in 50 μl of 1×first strand buffer (cDNA synthesis system; Life Technologies) containing a dNTP mix, [α-³²P]dCTP, dithiothreitol, and MMLV reverse transcriptase (Stratagene), and incubated at 42° C. for 1-2 hours. After incubation, the probe is diluted with 42 μl dH₂O, heated to 95° C. for 3 minutes, and cooled on ice. mRNA in the probe is removed by alkaline degradation. The probe is neutralized, and degraded mRNA and unincorporated nucleotides are removed using a PROBEQUANT G-50 Micro Column (Amersham Pharmacia Biotech). Probes can be labeled with fluorescent markers, Cy3-dCTP or Cy5-dCTP (Amersham Pharmacia Biotech), in place of the radionuclide [³²P]dCTP.

Hybridization

Hybridization is carried out at 65° C. in a hybridization buffer containing 0.5 M sodium phosphate (pH 7.2), 7% SDS, and 1 mM EDTA. After the blot is incubated in hybridization buffer at 65° C. for at least 2 hours, the buffer is replaced with 10 ml of fresh buffer containing the probe. After incubation at 65° C. for 18 hours, the hybridization buffer is removed, and the blot is washed sequentially under increasingly stringent conditions, up to 40 mM sodium phosphate, 1% SDS, 1 mM EDTA at 65° C. To detect signal produced by a radiolabeled probe hybridized on a membrane, the blot is exposed to a PHOSPHORIMAGER cassette (Molecular Dynamics, Sunnyvale Calif.), and the image is analyzed using IMAGEQUANT data analysis software (Molecular Dynamics). To detect signals produced by a fluorescent probe hybridized on a microarray, the blot is examined by confocal laser microscopy, and images are collected and analyzed using GEMTOOLS gene expression analysis software (Incyte Pharmaceuticals).

X Restriction Fragment Length Polymorphisms

Restriction fragment length polymorphisms (RFLPs) are created by using one or more restriction enzymes to cut DNA into fragments at specific recognition sites. The fragments and molecular size standards are separated using gel electrophoresis. Ethidium bromide staining is used to reveal the fragments under UV (260 nm) illumination. Fragment size differences between samples result from mutations or sequence rearrangements within restriction enzyme recognition sites. RFLP markers are selected by examining these differences (Paterson, A. H. et al. (1988) Nature 335:721-726). Alternative DNA fragments include RAPDs (Welsh, J. and McClelland, M. (1990) Nucleic Acids Res. 18:7213-7218), microsatellites (also called simple sequence repeats; Akkaya, M. S. et al. (1992) Genetics 132:1131-1139) or amplified fragment length polymorphisms (AFLPs; Nandi, S. et al. (1997); Mol. Gen. Genet. 255:1-8). Any of these DNA fragments may be mapped onto a chromosomal map, and all are chosen and used to study multigene traits or quantitative trait loci (QTL) at the intraspecific level or among closely related taxa.

XI Peptide Expression

Expression of a corn peptide is accomplished by transforming the multifunctional vector, PSPORT (Life Technologies) or PINCY (Incyte Pharmaceuticals), containing the sequence encoding the peptide into E. coli. A transfected colony is cultured and induced with isopropyl beta-D-thiogalactopyranoside (IPTG) using standard methods. The signal sequence resident in the vector directs the secretion of the peptide into the bacterial growth media. Purification of the peptide using polyacrylamide gel electrophoresis will provide peptide for antibody induction or for use in various assays.

XII Production of Antibodies

The amino acid sequence encoded by a cdp is analyzed using LASERGENE Navigator (DNASTAR) to determine regions of high immunogenicity. An oligopeptide of about 15 residues is synthesized using a Peptide Synthesizer Model 431A (Perkin Elmer) using Fmoc-chemistry and coupled to KLH (Sigma-Aldrich) by reaction with M-maleimidobenzoyl-N-hydroxysuccinimide ester (Ausubel, supra). If necessary, a cysteine may be introduced at the N-terminus of the oligopeptide to permit coupling to KLH. Rabbits are immunized with the oligopeptide-KLH complex in complete Freund's adjuvant. The resulting antisera are tested for antipeptide activity, for example, by binding the peptide to plastic, blocking with 1% BSA, reacting with rabbit antisera, washing, and reacting with radioiodinated goat anti-rabbit IgG.

XIII Two-Hybrid Screen

A yeast two-hybrid system such as MATCHMAKER LexA Two-Hybrid system (Clontech Laboratories, Palo Alto Calif.) is used to screen for peptides which bind CDPs. A nucleotide encoding a CDP is inserted into the multiple cloning site of a pLexA vector, ligated, and transformed into E. coli. cDNA, prepared from mRNA, is directionally inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ] using a polyethylene glycol/lithium acetate protocol. The transformed yeast cells are plated on synthetic dropout (SD) media lacking histidine (-His), tryptophan (-Trp), and uracil (-Ura), and incubated at 30° C. until colonies may be easily counted. The colonies are pooled in a minimal volume of 1×TE (pH 7.5), replated on SD/-His/-Leu/-Trp/-Ura media supplemented with 2% galactose (Gal), 1% raffinose (Raf), and 80 mg/ml X-Gal (X-Gal), and subsequently examined for growth of blue colonies. Interaction between expressed CDP and cDNA fusion proteins activates expression of a LEU2 reporter gene in EGY48 and produces colony growth on media lacking leucine (-Leu). Interaction also activates expression of β-galactosidase from the p8op-lacZ reporter construct which produces blue color in colonies grown on X-Gal.

Positive interactions between expressed CDP and cDNA fusion proteins can be verified by isolating individual positive colonies and growing them in SD/-Trp/-Ura liquid medium for 1-2 days at 30° C. A sample of the culture is plated on SD/-Trp/-Ura media and incubated at 30° C. until colonies appear. A sample of 20-30 colonies is identically arranged on SD/-Trp/-Ura and SD/-His/-Trp/-Ura plates. Colonies that grow on SD containing histidine but not on media lacking histidine have lost the pLexA plasmid. The histidine-requiring colonies are grown on SD/Gal/Raf/X-Gal/-Trp/-Ura, and white colonies are isolated and propagated. The pB42AD-cDNA plasmid, which contains a polynucleotide encoding a protein which physically interacts with a CDP, can be isolated from the yeast cells and characterized.

XIV Significant Sequences and Their Uses

The biological activity of polypeptides encoded by the cdps is based in part on a comparison between nucleic acid sequences in the Sequence Listing and reference or homologous sequences from GenBank which encode polypeptides of known function or activity. The biological properties and potential uses of polypeptides encoded by the cdps are based in part upon the biological properties of their known homologs.

Incyte Clone No. 700161207H1 is a nonexact homolog of GenBank GI No. g1483218, which encodes AWI 31, a gene specifically induced by wounding in Arabidopsis thaliana (Yang, K. Y. et al., 1997, Mol. Cells 7:131-135).

Wound-inducible mRNAs were isolated from Arabidopsis thaliana tissues harvested at short intervals after wounding. The mRNAs were used to produce cDNA clones which were classified into two groups according to when they were expressed. For nine clones, mRNA expression had increased within 1 to 1.5 hours after wounding, then declined. One clone, AWI 31, represented steady expression reaching maximum level at 2.5 hours. Yang et al. found that the AWI 31 gene had an open reading frame that predicted a protein of 386 amino acids and showed no significant homology to other known proteins. Northern hybridization using the cDNA revealed that the gene was not affected by other environmental stresses such as drought, high salt, low temperature, or a DPE herbicide treatment. These results suggest that the cDNA clone, AWI 31, was induced specifically by wounding.

Incyte Clone No. 700161207H1 is defined as a nonexact but functionally related homolog based on a product score of 22 and a log-likelihood value of −13 as shown in TABLE 1. Incyte Clone No. 700161207H1 can be used as a marker of damage/wounding after adverse environmental perturbation that could affect growth and yield.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

What is claimed is:

1. A purified corn seedling-derived polynucleotide comprising a nucleic acid sequence selected from the Sequence Listing or a complement of the nucleic acid sequence.

2. A composition for the detection of altered expression of corn seedling-derived polynucleotides comprising at least one of the nucleic acid sequences of claim 1.

3. A corn seedling-derived polynucleotide selected from the following nucleic acid sequences of claim 1: SEQ ID NO: 3537 (700162463H1), SEQ ID NO: 1161 (700158211H1), SEQ ID NO: 949 (700157856H1), SEQ ID NO: 3199 (700161822H1), SEQ ID NO: 1744 (700159434H1), SEQ ID NO: 1183 (700158258H1), SEQ ID NO: 377 (700156836H1), SEQ ID NO: 2501 (700160693H1), SEQ ID NO: 2527 (700160740H1), SEQ ID NO: 698 (700157394H1), SEQ ID NO: 2335 (700160435H1), SEQ ID NO: 1988 (700159867H1), SEQ ID NO: 2583 (700160844H1), SEQ ID NO: 585 (700157190H1), SEQ ID NO: 237 (700156608H1), SEQ ID NO: 2719 (700161045H1), SEQ ID NO: 266 (700156661H1), SEQ ID NO: 3021 (700161544H1), SEQ ID NO: 2040 (700159955H1), SEQ ID NO: 2224 (700160253H1), SEQ ID NO: 2761 (700161134H1), SEQ ID NO: 1166 (700158229H1), SEQ ID NO: 1164 (700158222H1), SEQ ID NO: 2479 (700160669H1), SEQ ID NO: 215 (700156563H1), SEQ ID NO: 2448 (700160623H1), SEQ ID NO: 2207 (700160227H1), SEQ ID NO: 2626 (700160906H1), SEQ ID NO: 1339 (700158532H1), SEQ ID NO: 2726 (700161054H1), SEQ ID NO: 336 (700156768H1), SEQ ID NO: 1963 (700159826H1), SEQ ID NO: 2285 (700160357H1), SEQ ID NO: 1969 (700159839H1), SEQ ID NO: 471 (700156989H1), SEQ ID NO: 3118 (700161689H1), SEQ ID NO: 2475 (700160665H1), SEQ ID NO: 2731 (700161064H1), SEQ ID NO: 1695 (700159330H1), SEQ ID NO: 35 (700142454H1), SEQ ID NO: 79 (700142522H1), SEQ ID NO: 2086 (700160030H1), SEQ ID NO: 2000 (700159882H1), SEQ ID NO: 1001 (700157954H1), SEQ ID NO: 1302 (700158460H1), SEQ ID NO: 1205 (700158305H1), SEQ ID NO: 2849 (700161279H1), SEQ ID NO: 2138 (700160112H1), SEQ ID NO: 224 (700156580H1), SEQ ID NO: 385 (700156847H1), SEQ ID NO: 3541 (700162468H1), SEQ ID NO: 3526 (700162448H1), SEQ ID NO: 2137 (700160111H1), SEQ ID NO: 1197 (700158287H1), SEQ ID NO: 2109 (700160067H1), SEQ ID NO: 1902 (700159726H1), SEQ ID NO: 2578 (700160832H1), SEQ ID NO: 1044 (700158022H1), SEQ ID NO: 956 (700157865H1), SEQ ID NO: 149 (700156439H1), SEQ ID NO: 1548 (700159028H1), SEQ ID NO: 3030 (700161557H1), SEQ ID NO: 2128 (700160091H1), SEQ ID NO: 3025 (700161550H1), SEQ ID NO: 195 (700156524H1), SEQ ID NO: 1385 (700158604H1), SEQ ID NO: 1091 (700158090H1), SEQ ID NO: 1517 (700158965H1), SEQ ID NO: 1439 (700158806H1), SEQ ID NO: 2755 (700161125H1), SEQ ID NO: 1449 (700158823H1), SEQ ID NO: 107 (700142566H1), SEQ ID NO: 2916 (700161388H1), SEQ ID NO: 3160 (700161766H1), SEQ ID NO: 3001 (700161515H1), SEQ ID NO: 1940 (700159780H1), SEQ ID NO: 2918 (700161390H1), SEQ ID NO: 426 (700156915H1), SEQ ID NO: 1024 (700157988H1), SEQ ID NO: 2468 (700160654H1), SEQ ID NO: 1886 (700159694H1), SEQ ID NO: 1160 (700158208H1), SEQ ID NO: 411 (700156889H1), SEQ ID NO: 1570 (700159068H1), SEQ ID NO: 3371 (700162137H1), SEQ ID NO: 1942 (700159783H1), SEQ ID NO: 3132 (700161719H1), SEQ ID NO: 138 (700156425H1), SEQ ID NO: 2870 (700161314H1), SEQ ID NO: 2624 (700160904H1), SEQ ID NO: 3518 (700162435H1), SEQ ID NO: 2931 (700161413H1), SEQ ID NO: 1649 (700159219H1), SEQ ID NO: 2477 (700160667H1), SEQ ID NO: 1513 (700158949H1), SEQ ID NO: 3340 (700162062H1), SEQ ID NO: 884 (700157746H1), SEQ ID NO: 1701 (700159340H1), SEQ ID NO: 2480 (700160671H1), SEQ ID NO: 938 (700157837H1), SEQ ID NO: 2834 (700161257H1), SEQ ID NO: 1020 (700157981H1), SEQ ID NO: 2272 (700160340H1), SEQ ID NO: 2055 (700159981H1), SEQ ID NO: 489 (700157022H1), SEQ ID NO: 1294 (700158449H1), SEQ ID NO: 1597 (700159125H1), SEQ ID NO: 1172 (700158240H1), SEQ ID NO: 1243 (700158366H1), SEQ ID NO: 2482 (700160674H1), SEQ ID NO: 2544 (700160768H1), SEQ ID NO: 2862 (700161296H1), SEQ ID NO: 2704 (700161024H1), SEQ ID NO: 2369 (700160492H1), SEQ ID NO: 3242 (700161894H1), SEQ ID NO: 2948 (700161439H1), SEQ ID NO: 1315 (700158484H1), SEQ ID NO: 2700 (700161016H1), SEQ ID NO: 1436 (700158695H1), SEQ ID NO: 1996 (700159877H1), SEQ ID NO: 172 (700156474H1), SEQ ID NO: 1787 (700159515H1), SEQ ID NO: 1929 (700159766H1), SEQ ID NO: 3601 (700162584H1), SEQ ID NO: 1615 (700159167H1), SEQ ID NO: 852 (700157686H1), SEQ ID NO: 2267 (700160335H1), SEQ ID NO: 2887 (700161344H1), SEQ ID NO: 803 (700157615H1), SEQ ID NO: 1878 (700159679H1), SEQ ID NO: 2026 (700159930H1), SEQ ID NO: 1250 (700158376H1), SEQ ID NO: 573 (700157172H1), SEQ ID NO: 105 (700142562H1), SEQ ID NO: 2205 (700160225H1), SEQ ID NO: 2389 (700160522H1), SEQ ID NO: 2876 (700161326H1), SEQ ID NO: 62 (700142492H1), SEQ ID NO: 2469 (700160658H1), SEQ ID NO: 1206 (700158310H1), SEQ ID NO: 666 (700157334H1), SEQ ID NO: 179 (700156486H1), SEQ ID NO: 1378 (700158590H1), SEQ ID NO: 3316 (700162025H1), SEQ ID NO: 578 (700157181H1), SEQ ID NO: 2433 (700160601H1), SEQ ID NO: 865 (700157712H1), SEQ ID NO: 1726 (700159386H1), SEQ ID NO: 3165 (700161772H1), SEQ ID NO: 1409 (700158643H1), SEQ ID NO: 2183 (700160186H1), SEQ ID NO: 2977 (700161476H1), SEQ ID NO: 1985 (700159386H1), SEQ ID NO: 768 (700157547H1), SEQ ID NO: 1499 (700158924H1), SEQ ID NO: 1404 (700158632H1), SEQ ID NO: 374 (700156831H1), SEQ ID NO: 1908 (700159733H1), SEQ ID NO: 1405 (700158636H1), SEQ ID NO: 1543 (700159020H1), SEQ ID NO: 2139 (700160115H1), SEQ ID NO: 1258 (700158387H1), SEQ ID NO: 3344 (700162066H1), SEQ ID NO: 3580 (700162539H1), SEQ ID NO: 1830 (700159602H2), SEQ ID NO: 3531 (700162455H1), SEQ ID NO: 515 (700157065H1), SEQ ID NO: 2723 (700161051H1), SEQ ID NO: 191 (700156519H1), SEQ ID NO: 3259 (700161925H1), SEQ ID NO: 622 (700157251H1), SEQ ID NO: 68 (700142503H1), SEQ ID NO: 32 (700142451H1), SEQ ID NO: 80 (700142523H1), SEQ ID NO: 2225 (700160259H1), SEQ ID NO: 37 (700142458H1), SEQ ID NO: 559 (700157147H1), SEQ ID NO: 581 (700157185H1), SEQ ID NO: 3042 (700161571H1), SEQ ID NO: 2789 (700161181H1), SEQ ID NO: 2692 (700161003H1), SEQ ID NO: 1493 (700158915H1), SEQ ID NO: 2431 (700160595H1), SEQ ID NO: 270 (700156667H1), SEQ ID NO: 2010 (700159905H1), SEQ ID NO: 3401 (700162185H1), SEQ ID NO: 3501 (700162392H1), SEQ ID NO: 307 (700156727H1), SEQ ID NO: 2830 (700161249H1), SEQ ID NO: 3464 (700162321H1), SEQ ID NO: 1034 (700158010H1), SEQ ID NO: 2573 (700160826H1), SEQ ID NO: 1657 (700159236H1), SEQ ID NO: 1646 (700159214H1), SEQ ID NO: 198 (700156531H1), SEQ ID NO: 151 (700156441H1), SEQ ID NO: 3257 (700161923H1), SEQ ID NO: 3553 (700162485H1), SEQ ID NO: 1839 (700159614H1), SEQ ID NO: 3056 (700161591H1), SEQ ID NO: 3320 (700162032H1), 15 SEQ ID NO: 629 (700157262H1), SEQ ID NO: 1889 (700159703H1), SEQ ID NO: 2628 (700160908H1), SEQ ID NO: 1670 (700159272H1), SEQ ID NO: 1274 (700158421H1), SEQ ID NO: 1039 (700158016H1), SEQ ID NO: 1975 (700159850H1), SEQ ID NO: 2951 (700161442H1), SEQ ID NO: 1042 (700158020H1), SEQ ID NO: 1521 (700158980H1), SEQ ID NO: 1119 (700158143H1), SEQ ID NO: 277 (700156677H1), SEQ ID NO: 519 (700157072H1), SEQ ID NO: 1422 (700158672H1), SEQ ID NO: 2315 (700160405H1), SEQ ID NO: 922 (700157810H1), SEQ ID NO: 1175 (700158245H1), SEQ ID NO: 1763 (700159461H1), SEQ ID NO: 701 (700157405H1), SEQ ID NO: 2540 (700160761H1), SEQ ID NO: 1157 (700158204H1), SEQ ID NO: 576 (700157177H1), SEQ ID NO: 356 (700156801H1), SEQ ID NO: 1375 (700158585H1), SEQ ID NO: 3146 (700161738H1), SEQ ID NO: 3150 (700161744H1), SEQ ID NO: 1464 (700158854H1), SEQ ID NO: 2963 (700161457H1), SEQ ID NO: 2213 (700160236H1), SEQ ID NO: 1128 (700158158H1), SEQ ID NO: 3528 (700162450H1), SEQ ID NO: 1730 (700159395H1), SEQ ID NO: 3168 (700161775H1), SEQ ID NO: 918 (700157804H1), SEQ ID NO: 1775 (700159482H1), SEQ ID NO: 2676 (700160978H1), SEQ ID NO: 1567 (700159064H1), SEQ ID NO: 2412 (700160563H1), SEQ ID NO: 1003 (700157957H1), SEQ ID NO: 109 (700142571H1), SEQ ID NO: 1589 (700159110H1), SEQ ID NO: 508 (700157053H1), SEQ ID NO: 354 (700156792H1), SEQ ID NO: 505 (700157045H1), SEQ ID NO: 1307 (700158470H1), SEQ ID NO: 600 (700157219H1), SEQ ID NO: 1121 (700158145H1), SEQ ID NO: 1377 (700158588H1), SEQ ID NO: 2710 (700161034H1), SEQ ID NO: 1843 (700159620H1), SEQ ID NO: 998 (700157949H1), SEQ ID NO: 3564 (700162513H1), SEQ ID NO: 2708 (700161029H1), SEQ ID NO: 796 (700157593H1), SEQ ID NO: 1346 (700158541H1), SEQ ID NO: 2594 (700160860H1), SEQ ID NO: 898 (700157768H1), SEQ ID NO: 3384 (700162163H1), SEQ ID NO: 1552 (700159036H1), SEQ ID NO: 1554 (700159038H1), SEQ ID NO: 1712 (700159356H1), SEQ ID NO: 3094 (700161653H1), SEQ ID NO: 3525 (700162447H1), SEQ ID NO: 1858 (700159650H1), SEQ ID NO: 3428 (700162243H1), SEQ ID NO: 1997 (700159879H1), SEQ ID NO: 2451 (700160628H1), SEQ ID NO: 429 (700156922H1), SEQ ID NO: 1906 (700159731H1), SEQ ID NO: 2660 (700160954H1), SEQ ID NO: 1557 (700159046H1), SEQ ID NO: 2672 (700160972H1), SEQ ID NO: 1066 (700158055H1), SEQ ID NO: 3449 (700162285H1), SEQ ID NO: 1693 (700159327H1), SEQ ID NO: 601 (700157222H1), SEQ ID NO: 3527 (700162449H1), SEQ ID NO: 85 (700142532H1), SEQ ID NO: 2062 (700159989H1), SEQ ID NO: 1862 (700159656H1), SEQ ID NO: 3544 (700162472H1), SEQ ID NO: 279 (700156681H1), SEQ ID NO: 2842 (700161267H1), SEQ ID NO: 1210 (700158315H1), SEQ ID NO: 2456 (700160640H1), SEQ ID NO: 2620 (700160893H1), SEQ ID NO: 724 (700157446H1), SEQ ID NO: 615 (700157240H1), SEQ ID NO: 1694 (700159328H1), SEQ ID NO: 500 (700157039H1), SEQ ID NO: 2781 (700161170H1), SEQ ID NO: 2909 (700161381H1), SEQ ID NO: 436 (700156929H1), SEQ ID NO: 1036 (700158013H1), SEQ ID NO: 1736 (700159416H1), SEQ ID NO: 2569 (700160819H1), SEQ ID NO: 3313 (700162020H1), SEQ ID NO: 1073 (700158065H1), SEQ ID NO: 1990 (700159869H1), SEQ ID NO: 88 (700142539H1), SEQ ID NO: 1704 (700159347H1), SEQ ID NO: 1158 (700158205H1), SEQ ID NO: 3586 (700162548H1), SEQ ID NO: 1901 (700159724H1), SEQ ID NO: 1994 (700159875H1), SEQ ID NO: 3142 (700161734H1), SEQ ID NO: 2907 (700161378H1), SEQ ID NO: 1937 (700159776H1), SEQ ID NO: 2767 (700161143H1), SEQ ID NO: 2032 (700159941H1), SEQ ID NO: 3099 (700161661H1), SEQ ID NO: 2828 (700161245H1), SEQ ID NO: 437 (700156933H1), SEQ ID NO: 2985 (700161490H1), SEQ ID NO: 2499 (700160691H1), SEQ ID NO: 2336 (700160436H1), SEQ ID NO: 1074 (700158066H1), SEQ ID NO: 2073 (700160007H1), SEQ ID NO: 2956 (700161448H1), SEQ ID NO: 1089 (700158086H1), SEQ ID NO: 2195 (700160211H1), SEQ ID NO: 1741 (700159425H1), SEQ ID NO: 607 (700157229H1), SEQ ID NO: 2840 (700161265H1), SEQ ID NO: 2649 (700160942H1), SEQ ID NO: 1221 (700158332H1), SEQ ID NO: 1813 (700159568H1), SEQ ID NO: 1805 (700159552H1), SEQ ID NO: 285 (700156691H1), SEQ ID NO: 294 (700156709H1), SEQ ID NO: 3291 (700161979H1), SEQ ID NO: 2634 (700160921H1), SEQ ID NO: 3039 (700161567H1), SEQ ID NO: 3054 (700161589H1), SEQ ID NO: 453 (700156964H1), SEQ ID NO: 2681 (700160985H1), SEQ ID NO: 937 (700157836H1), SEQ ID NO: 1703 (700159346H1), SEQ ID NO: 2829 (700161247H1), SEQ ID NO: 3120 (700161693H1), SEQ ID NO: 552 (700157137H1), SEQ ID NO: 1431 (700158688H1), SEQ ID NO: 1806 (700159554H1), SEQ ID NO: 1814 (700159570H1), SEQ ID NO: 3308 (700162013H1), SEQ ID NO: 167 (700156463H1), SEQ ID NO: 2059 (700159986H1), SEQ ID NO: 242 (700156618H1), SEQ ID NO: 1256 (700158383H1), SEQ ID NO: 744 (700157489H1), SEQ ID NO: 1895 (700159716H1), SEQ ID NO: 440 (700156938H1), SEQ ID NO: 3217 (700161849H1), SEQ ID NO: 2831 (700161251H1), SEQ ID NO: 1015 (700157975H1), SEQ ID NO: 1366 (700158568H1), SEQ ID NO: 314 (700156740H1), SEQ ID NO: 2188 (700160195H1), SEQ ID NO: 84 (700142528H1), SEQ ID NO: 3069 (700161614H1), SEQ ID NO: 2952 (700161443H1), SEQ ID NO: 53 (700142478H1), SEQ ID NO: 1778 (700159492H1), SEQ ID NO: 1881 (700159685H1), SEQ ID NO: 1907 (700159732H1), SEQ ID NO: 614 (700157239H1), SEQ ID NO: 3395 (700162178H1), SEQ ID NO: 1312 (700158481H1), SEQ ID NO: 243 (700156621H1), SEQ ID NO: 1131 (700158163H1), SEQ ID NO: 678 (700157359H1), SEQ ID NO: 1506 (700158934H1), SEQ ID NO: 2643 (700160932H1), SEQ ID NO: 2593 (700160857H1), SEQ ID NO: 142 (700156430H1), SEQ ID NO: 2371 (700160494H1), SEQ ID NO: 925 (700157817H1), SEQ ID NO: 3041 (700161570H1), SEQ ID NO: 3050 (700161583H1), SEQ ID NO: 3079 (700161637H1), SEQ ID NO: 1245 (700158369H1), SEQ ID NO: 2309 (700160391H1), SEQ ID NO: 2713 (700161037H1), SEQ ID NO: 1289 (700158440H1), SEQ ID NO: 635 (700157268H1), SEQ ID NO: 653 (700157309H1), SEQ ID NO: 244 (700156623H1), SEQ ID NO: 338 (700156771H1), SEQ ID NO: 1406 (700158637H1), SEQ ID NO: 2941 (700161430H1), SEQ ID NO: 2516 (700160725H1), SEQ ID NO: 1945 (700159791H1), SEQ ID NO: 1485 (700158902H1), SEQ ID NO: 3045 (700161576H1), SEQ ID NO: 1271 (700158417H1), SEQ ID NO: 2786 (700161176H1), SEQ ID NO: 156 (700156447H1), SEQ ID NO: 1367 (700158574H1), SEQ ID NO: 3169 (700161776H1), SEQ ID NO: 2076 (700160012H1), SEQ ID NO: 3253 (700161917H1), SEQ ID NO: 950 (700157857H1), SEQ ID NO: 1237 (700158356H1), SEQ ID NO: 2063 (700159990H1), SEQ ID NO: 3574 (700162527H1), SEQ ID NO: 580 (700157183H1), SEQ ID NO: 2034 (700159943H1), SEQ ID NO: 892 (700157757H1), SEQ ID NO: 1096 (700158104H1), SEQ ID NO: 1883 (700159691H1), SEQ ID NO: 2966 (700161460H1), SEQ ID NO: 1632 (700159193H1), SEQ ID NO: 1811 (700159565H1), SEQ ID NO: 1803 (700159549H1), SEQ ID NO: 2390 (700160523H1), SEQ ID NO: 2329 (700160426H1), SEQ ID NO: 2835 (700161259H1), SEQ ID NO: 1428 (700158681H1), SEQ ID NO: 2016 (700159915H1), SEQ ID NO: 3487 (700162367H1), SEQ ID NO: 2699 (700161014H1), SEQ ID NO: 2694 (700161006H1), SEQ ID NO: 163 (700156456H1), SEQ ID NO: 2302 (700160381H1), SEQ ID NO: 23 (700142438H1), SEQ ID NO: 1720 (700159376H1), SEQ ID NO: 1519 (700158975H1), SEQ ID NO: 569 (700157166H1), SEQ ID NO: 3015 (700161536H1), SEQ ID NO: 521 (700157076H1), SEQ ID NO: 736 (700157474H1), SEQ ID NO: 626 (700157256H1), SEQ ID NO: 1846 (700159627H1), SEQ ID NO: 1642 (700159209H1), SEQ ID NO: 2391 (700160526H1), SEQ ID NO: 2410 (700160559H1), SEQ ID NO: 2061 (700159988H1), SEQ ID NO: 413 (700156891H1), SEQ ID NO: 1072 (700158064H1), SEQ ID NO: 1038 (700158015H1), SEQ ID NO: 1854 (700159641H1), SEQ ID NO: 143 (700156431H1), SEQ ID NO: 81 (700142524H1), SEQ ID NO: 3111 (700161679H1), SEQ ID NO: 2806 (700161213H1), SEQ ID NO: 139 (700156426H1), SEQ ID NO: 43 (700142466H1), SEQ ID NO: 185 (700156495H1), SEQ ID NO: 2524 (700160737H1), SEQ ID NO: 64 (700142494H1), SEQ ID NO: 3171 (700161778H1), SEQ ID NO: 2707 (700161028H1), SEQ ID NO: 2033 (700159942H1), SEQ ID NO: 639 (700157276H1), SEQ ID NO: 532 (700157093H1), SEQ ID NO: 3155 (700161756H1), SEQ ID NO: 1338 (700158531H1), SEQ ID NO: 575 (700157176H1), SEQ ID NO: 1177 (700158248H1), SEQ ID NO: 1099 (700158107H1), SEQ ID NO: 1861 (700159654H1), SEQ ID NO: 1832 (700159606H1), SEQ ID NO: 829 (700157650H1), SEQ ID NO: 878 (700157737H1), SEQ ID NO: 1443 (700158812H1), SEQ ID NO: 329 (700156761H1), SEQ ID NO: 3227 (700161866H1), SEQ ID NO: 851 (700157685H1), SEQ ID NO: 2728 (700161056H1), SEQ ID NO: 2854 (700161284H1), SEQ ID NO: 3547 (700162477H1), SEQ ID NO: 2393 (700160529H1), SEQ ID NO: 323 (700156754H1), SEQ ID NO: 1407 (700158638H1), SEQ ID NO: 187 (700156506H1), SEQ ID NO: 441 (700156940H1), SEQ ID NO: 1904 (700159728H1), SEQ ID NO: 1386 (700158605H1), SEQ ID NO: 3323 (700162037H1), SEQ ID NO: 2576 (700160830H1), SEQ ID NO: 1283 (700158434H1), SEQ ID NO: 2048 (700159970H1), SEQ ID NO: 2123 (700160085H1), SEQ ID NO: 2305 (700160385H1), SEQ ID NO: 1191 (700158278H1), SEQ ID NO: 1420 (700158669H1), SEQ ID NO: 335 (700156767H1), SEQ ID NO: 2889 (700161346H1), SEQ ID NO: 798 (700157601H1), SEQ ID NO: 976 (700157911H1), SEQ ID NO: 104 (700142561H1), SEQ ID NO: 1052 (700158034H1), SEQ ID NO: 1347 (700158542H1), SEQ ID NO: 1653 (700159226H1), SEQ ID NO: 958 (700157868H1), SEQ ID NO: 2252 (700160313H1), SEQ ID NO: 2181 (700160184H1), SEQ ID NO: 971 (700157895H1), SEQ ID NO: 2492 (700160684H1), SEQ ID NO: 1043 (700158021H1), SEQ ID NO: 12 (700142422H1), SEQ ID NO: 592 (700157207H1), SEQ ID NO: 3163 (700161770H1), SEQ ID NO: 3331 (700162049H1), SEQ ID NO: 2591 (700160855H1), SEQ ID NO: 2153 (700160140H1), SEQ ID NO: 1917 (700159748H1), SEQ ID NO: 2824 (700161241H1), SEQ ID NO: 3407 (700162206H1), SEQ ID NO: 1453 (700158832H1), SEQ ID NO: 2747 (700161102H1), SEQ ID NO: 2801 (700161206H1), SEQ ID NO: 1558 (700159047H1), SEQ ID NO: 2888 (700161345H1), SEQ ID NO: 1681 (700159307H1), SEQ ID NO: 2330 (700160427H1), SEQ ID NO: 1607 (700159151H1), SEQ ID NO: 1635 (700159196H1), SEQ ID NO: 2115 (700160076H1), SEQ ID NO: 656 (700157319H1), SEQ ID NO: 888 (700157751H1), SEQ ID NO: 711 (700157424H1), SEQ ID NO: 2307 (700160387H1), SEQ ID NO: 145 (700156433H1), SEQ ID NO: 1734 (700159411H1), SEQ ID NO: 342 (700156776H1), SEQ ID NO: 1842 (700159619H1), SEQ ID NO: 1360 (700158558H1), SEQ ID NO: 799 (700157605H1), SEQ ID NO: 1759 (700159454H1), SEQ ID NO: 2486 (700160678H1), SEQ ID NO: 3194 (700161816H1), SEQ ID NO: 1457 (700158839H1), SEQ ID NO: 2666 (700160964H1), SEQ ID NO: 3509 (700162417H1), SEQ ID NO: 2430 (700160593H1), SEQ ID NO: 446 (700156951H1), SEQ ID NO: 3273 (700161948H1), SEQ ID NO: 822 (700157642H1), SEQ ID NO: 1981 (700159857H1), SEQ ID NO: 1146 (700158184H1), SEQ ID NO: 3594 (700162570H1), SEQ ID NO: 2179 (700160180H1), SEQ ID NO: 1195 (700158285H1), SEQ ID NO: 3489 (700162376H1), SEQ ID NO: 2192 (700160204H1), SEQ ID NO: 3023 (700161547H1), SEQ ID NO: 363 (700156813H1), SEQ ID NO: 2498 (700160690H1), SEQ ID NO: 2491 (700160683H1), SEQ ID NO: 1875 (700159673H1), SEQ ID NO: 127 (700156407H1), SEQ ID NO: 1381 (700158596H1), SEQ ID NO: 1630 (700159187H1), SEQ ID NO: 3325 (700162042H1), SEQ ID NO: 3444 (700162277H1), SEQ ID NO: 1705 (700159348H1), SEQ ID NO: 2533 (700160750H1), SEQ ID NO: 1986 (700159864H1), SEQ ID NO: 2348 (700160459H1), SEQ ID NO: 3019 (700161541H1), SEQ ID NO: 2557 (700160795H1), SEQ ID NO: 3212 (700161842H1), SEQ ID NO: 3202 (700161826H1), SEQ ID NO: 1745 (700159435H1), SEQ ID NO: 188 (700156511H1), SEQ ID NO: 964 (700157879H1), SEQ ID NO: 3470 (700162338H1), SEQ ID NO: 3603 (700162591H1), SEQ ID NO: 2500 (700160692H1), SEQ ID NO: 2775 (700161157H1), SEQ ID NO: 2778 (700161166H1), SEQ ID NO: 1740 (700159423H1), SEQ ID NO: 1576 (700159081H1), SEQ ID NO: 3508 (700162414H1), SEQ ID NO: 3484 (700162364H1), SEQ ID NO: 1054 (700158037H1), SEQ ID NO: 4316 (700454828H1), SEQ ID NO: 4594 (700455401H1), SEQ ID NO: 4100 (700454421H1), SEQ ID NO: 5528 (700457192H1), SEQ ID NO: 5760 (700457654H1), SEQ ID NO: 3797 (700453825H1), SEQ ID NO: 5257 (700456691H1), SEQ ID NO: 3788 (700453810H1), SEQ ID NO: 4097 (700454418H1), SEQ ID NO: 5731 (700457589H1), SEQ ID NO: 5009 (700456178H1), SEQ ID NO: 4972 (700456102H1), SEQ ID NO: 5423 (700457018H1), SEQ ID NO: 4519 (700455175H1), SEQ ID NO: 4729 (700455675H1), SEQ ID NO: 5640 (700457388H1), SEQ ID NO: 6092 (700458353H1), SEQ ID NO: 4157 (700454513H1), SEQ ID NO: 6103 (700458378H1), SEQ ID NO: 3821 (700453864H1), SEQ ID NO: 4968 (700456094H1), SEQ ID NO: 4292 (700454771H1), SEQ ID NO: 5425 (700457023H1), SEQ ID NO: 5154 (700456454H1), SEQ ID NO: 5445 (700457055H1), SEQ ID NO: 5146 (700456444H1), SEQ ID NO: 4643 (700455525H1), SEQ ID NO: 5267 (700456715H1), SEQ ID NO: 4380 (700454949H1), SEQ ID NO: 3883 (700453981H1), SEQ ID NO: 5847 (700457846H1), SEQ ID NO: 4889 (700455957H1), SEQ ID NO: 5239 (700456657H1), SEQ ID NO: 5072 (700456315H1), SEQ ID NO: 6018 (700458195H1), SEQ ID NO: 5205 (700456575H1), SEQ ID NO: 5826 (700457804H1), SEQ ID NO: 4475 (700455102H1), SEQ ID NO: 5429 (700457030H1), SEQ ID NO: 5452 (700457062H1), SEQ ID NO: 5453 (700457063H1), SEQ ID NO: 4131 (700454465H1), SEQ ID NO: 5538 (700457213H1), SEQ ID NO: 5540 (700457215H1), SEQ ID NO: 5433 (700457035H1), SEQ ID NO: 3984 (700454206H1), SEQ ID NO: 5369 (700456891H1), SEQ ID NO: 5766 (700457667H1), SEQ ID NO: 5699 (700457519H1), SEQ ID NO: 5698 (700457518H1), SEQ ID NO: 5907 (700457953H1), SEQ ID NO: 5829 (700457808H1), SEQ ID NO: 5872 (700457887H1), SEQ ID NO: 4247 (700454678H1), SEQ ID NO: 5225 (700456633H1), SEQ ID NO: 4823 (700455851H1), SEQ ID NO: 4083 (700454394H1), SEQ ID NO: 4084 (700454395H1), SEQ ID NO: 4030 (700454295H1), SEQ ID NO: 5636 (700457380H1), SEQ ID NO: 4784 (700455780H1), SEQ ID NO: 5925 (700457993H1), SEQ ID NO: 6119 (700458416H1), SEQ ID NO: 6124 (700458424H1), SEQ ID NO: 4463 (700455081H1), SEQ ID NO: 5554 (700457243H1), SEQ ID NO: 5497 (700457136H1), SEQ ID NO: 5602 (700457326H1), SEQ ID NO: 5651 (700457411H1), SEQ ID NO: 4522 (700455178H1), SEQ ID NO: 5647 (700457403H1), SEQ ID NO: 5426 (700457025H1), SEQ ID NO: 5395 (700456958H1), SEQ ID NO: 5422 (700457017H1), SEQ ID NO: 6127 (700458428H1), SEQ ID NO: 6121 (700458420H1), SEQ ID NO: 6128 (700458429H1), SEQ ID NO: 4915 (700456004H1), SEQ ID NO: 5183 (700456520H1), SEQ ID NO: 5179 (700456505H1), SEQ ID NO: 5480 (700457107H1), SEQ ID NO: 6070 (700458311H1), SEQ ID NO: 4263 (700454707H1), SEQ ID NO: 4325 (700454841H1), SEQ ID NO: 5552 (700457241H1), SEQ ID NO: 6241 (700458630H1), SEQ ID NO: 4853 (700455908H1), SEQ ID NO: 5088 (700456339H1), SEQ ID NO: 5082 (700456331H1), SEQ ID NO: 4419 (700455012H1), SEQ ID NO: 5131 (700456416H1), SEQ ID NO: 4417 (700455010H1), SEQ ID NO: 4137 (700454471H1), SEQ ID NO: 4786 (700455782H1), SEQ ID NO: 5221 (700456626H1), SEQ ID NO: 4095 (700454414H1), SEQ ID NO: 4453 (700455062H1), SEQ ID NO: 5253 (700456683H1), SEQ ID NO: 4588 (700455290H1), SEQ ID NO: 4456 (700455068H1), SEQ ID NO: 4428 (700455024H1), SEQ ID NO: 3825 (700453874H1), SEQ ID NO: 4686 (700455610H1), SEQ ID NO: 4711 (700455650H1), SEQ ID NO: 4067 (700454365H1), SEQ ID NO: 4436 (700455038H1), SEQ ID NO: 6010 (700458175H1), SEQ ID NO: 4256 (700454692H1), SEQ ID NO: 5474 (700457092H1), SEQ ID NO: 4656 (700455554H1), SEQ ID NO: 4832 (700455867H1), SEQ ID NO: 5744 (700457627H1), SEQ ID NO: 5226 (700456634H1), SEQ ID NO: 4529 (700455189H1), SEQ ID NO: 5471 (700457085H1), SEQ ID NO: 5451 (700457061H1), SEQ ID NO: 5828 (700457807H1), SEQ ID NO: 5771 (700457674H1), SEQ ID NO: 4957 (700456074H1), SEQ ID NO: 4985 (700456127H1), SEQ ID NO: 4981 (700456114H1), SEQ ID NO: 3971 (700454173H1), SEQ ID NO: 4060 (700454351H1), SEQ ID NO: 4734 (700455681H1), SEQ ID NO: 5136 (700456423H1), SEQ ID NO: 5680 (700457471H1), SEQ ID NO: 5980 (700458123H1), SEQ ID NO: 3940 (700454119H1), SEQ ID NO: 3866 (700453958H1), SEQ ID NO: 5861 (700457867H1), SEQ ID NO: 3836 (700453895H1), SEQ ID NO: 5458 (700457072H1), SEQ ID NO: 4366 (700454923H1), SEQ ID NO: 4211 (700454614H1), SEQ ID NO: 6273 (700458684H1), SEQ ID NO: 5875 (700457891H1), SEQ ID NO: 5140 (700456433H1), SEQ ID NO: 5986 (700458136H1), SEQ ID NO: 6262 (700458668H1), SEQ ID NO: 5247 (700456675H1), SEQ ID NO: 5721 (700457565H1), SEQ ID NO: 4531 (700455195H1), SEQ ID NO: 5577 (700457273H1), SEQ ID NO: 4399 (700454984H1), SEQ ID NO: 6072 (700458313H1), SEQ ID NO: 4442 (700455048H1), SEQ ID NO: 4078 (700454381H1), SEQ ID NO: 4751 (700455713H1), SEQ ID NO: 5790 (700457717H1), SEQ ID NO: 5406 (700456977H1), SEQ ID NO: 4532 (700455196H1), SEQ ID NO: 4012 (700454262H1), SEQ ID NO: 5556 (700457245H1), SEQ ID NO: 5262 (700456707H1), SEQ ID NO: 4900 (700455974H1), SEQ ID NO: 5290 (700456752H1), SEQ ID NO: 3957 (700454145H1), SEQ ID NO: 6291 (700458826H1), SEQ ID NO: 6292 (700458827H1), SEQ ID NO: 4639 (700455517H1), SEQ ID NO: 6137 (700458443H1), SEQ ID NO: 4367 (700454925H1), SEQ ID NO: 4467 (700455085H1), SEQ ID NO: 5473 (700457090H1), SEQ ID NO: 5482 (700457115H1), SEQ ID NO: 4266 (700454717H1), SEQ ID NO: 4272 (700454725H1), SEQ ID NO: 4356 (700454909H1), SEQ ID NO: 4135 (700454469H1), SEQ ID NO: 5259 (700456694H1), SEQ ID NO: 5736 (700457604H1), SEQ ID NO: 5114 (700456381H1), SEQ ID NO: 5656 (700457419H1), SEQ ID NO: 4512 (700455167H1), SEQ ID NO: 4460 (700455077H1), SEQ ID NO: 4553 (700455237H1), SEQ ID NO: 4373 (700454937H1), SEQ ID NO: 4400 (700454985H1), SEQ ID NO: 5379 (700456917H1), SEQ ID NO: 5678 (700457466H1), SEQ ID NO: 5746 (700457629H1), SEQ ID NO: 5152 (700456452H1), SEQ ID NO: 5133 (700456418H1), SEQ ID NO: 4908 (700455987H1), SEQ ID NO: 5650 (700457409H1), SEQ ID NO: 6060 (700458287H1), SEQ ID NO: 6011 (700458178H1), SEQ ID NO: 4271 (700454724H1), SEQ ID NO: 5489 (700457124H1), SEQ ID NO: 5762 (700457658H1), SEQ ID NO: 4068 (700454366H1), SEQ ID NO: 4075 (700454377H1), SEQ ID NO: 3910 (700454049H1), SEQ ID NO: 4394 (700454970H1), SEQ ID NO: 5450 (700457060H1), SEQ ID NO: 5719 (700457561H1), SEQ ID NO: 5763 (700457660H1), SEQ ID NO: 4725 (700455669H1), SEQ ID NO: 4003 (700454241H1), SEQ ID NO: 5487 (700457122H1), SEQ ID NO: 5264 (700456711H1), SEQ ID NO: 5850 (700457850H1), SEQ ID NO: 5501 (700457141H1), SEQ ID NO: 4085 (700454396H1), SEQ ID NO: 4091 (700454409H1), SEQ ID NO: 4404 (700454989H1), SEQ ID NO: 5390 (700456948H1), SEQ ID NO: 5751 (700457638H1), SEQ ID NO: 4914 (700456003H1), SEQ ID NO: 6035 (700458234H1), SEQ ID NO: 6078 (700458322H1), SEQ ID NO: 5893 (700457927H1), SEQ ID NO: 5309 (700456784H1), SEQ ID NO: 4539 (700455212H1), SEQ ID NO: 5551 (700457240H1), SEQ ID NO: 5522 (700457174H1), SEQ ID NO: 5486 (700457121H1), SEQ ID NO: 4478 (700455106H1), SEQ ID NO: 4139 (700454474H1), SEQ ID NO: 5938 (700458026H1), SEQ ID NO: 6329 (700458889H1), SEQ ID NO: 5155 (700456455H1), SEQ ID NO: 5883 (700457911H1), SEQ ID NO: 3873 (700453966H1), SEQ ID NO: 4652 (700455543H1), SEQ ID NO: 4425 (700455021H1), SEQ ID NO: 4406 (700454992H1), SEQ ID NO: 6084 (700458339H1), SEQ ID NO: 3644 (700405359H1), SEQ ID NO: 5258 (700456692H1), SEQ ID NO: 4088 (700454405H1), SEQ ID NO: 3976 (700454186H1), SEQ ID NO: 4054 (700454337H1), SEQ ID NO: 4704 (700455637H1), SEQ ID NO: 3707 (700405462H1), SEQ ID NO: 6165 (700458510H1), SEQ ID NO: 6058 (700458285H1), SEQ ID NO: 4275 (700454729H1), SEQ ID NO: 3755 (700453757H1), SEQ ID NO: 5566 (700457256H1), SEQ ID NO: 4545 (700455226H1), SEQ ID NO: 4144 (700454483H1), SEQ ID NO: 5216 (700456614H1), SEQ ID NO: 4490 (700455127H1), SEQ ID NO: 5278 (700456729H1), SEQ ID NO: 5260 (700456695H1), SEQ ID NO: 5157 (700456458H1), SEQ ID NO: 5483 (700457116H1), SEQ ID NO: 4032 (700454303H1), SEQ ID NO: 5709 (700457540H1), SEQ ID NO: 4125 (700454456H1), SEQ ID NO: 4503 (700455152H1), SEQ ID NO: 4567 (700455262H1), SEQ ID NO: 5442 (700457051H1), SEQ ID NO: 5573 (700457266H1), SEQ ID NO: 5531 (700457202H1), SEQ ID NO: 4031 (700454302H1), SEQ ID NO: 5381 (700456920H1), SEQ ID NO: 6257 (700458655H1), SEQ ID NO: 4322 (700454836H1), SEQ ID NO: 6059 (700458286H1), SEQ ID NO: 5090 (700456344H1), SEQ ID NO: 4920 (700456014H1), SEQ ID NO: 4555 (700455240H1), SEQ ID NO: 6046 (700458257H1), SEQ ID NO: 3985 (700454209H1), SEQ ID NO: 3988 (700454213H1), SEQ ID NO: 5163 (700456469H1), SEQ ID NO: 5791 (700457720H1), SEQ ID NO: 4363 (700454917H1), SEQ ID NO: 4474 (700455101H1), SEQ ID NO: 4337 (700454859H1), SEQ ID NO: 4340 (700454864H1), SEQ ID NO: 3786 (700453808H1), SEQ ID NO: 3686 (700405427H1), SEQ ID NO: 5691 (700457504H1), SEQ ID NO: 4059 (700454350H1), SEQ ID NO: 4038 (700454313H1), SEQ ID NO: 4039 (700454314H1), SEQ ID NO: 5147 (700456445H1), SEQ ID NO: 4376 (700454942H1), SEQ ID NO: 3997 (700454229H1), SEQ ID NO: 3892 (700454004H1), SEQ ID NO: 5747 (700457630H1), SEQ ID NO: 4835 (700455873H1), SEQ ID NO: 3941 (700454121H1), SEQ ID NO: 3990 (700454221H1), SEQ ID NO: 5011 (700456186H1), SEQ ID NO: 5411 (700456986H1), SEQ ID NO: 5904 (700457948H1), SEQ ID NO: 5642 (700457392H1), SEQ ID NO: 4098 (700454419H1), SEQ ID NO: 5803 (700457750H1), SEQ ID NO: 4955 (700456069H1), SEQ ID NO: 5251 (700456679H1), SEQ ID NO: 6017 (700458194H1), SEQ ID NO: 4159 (700454516H1), SEQ ID NO: 4514 (700455169H1), SEQ ID NO: 5271 (700456720H1), SEQ ID NO: 4426 (700455022H1), SEQ ID NO: 4409 (700454996H1), SEQ ID NO: 4559 (700455247H1) SEQ ID NO: 5472 (700457088H1), SEQ ID NO: 3638 (700405352H1), SEQ ID NO: 5283 (700456741H1), SEQ ID NO: 5781 (700457692H1), SEQ ID NO: 5628 (700457367H1), SEQ ID NO: 5076 (700456324H1), SEQ ID NO: 5890 (700457924H1), SEQ ID NO: 4299 (700454785H1), SEQ ID NO: 3740 (700453730H1), SEQ ID NO: 5761 (700457656H1), SEQ ID NO: 5765 (700457663H1), SEQ ID NO: 4699 (700455630H1), SEQ ID NO: 4346 (700454880H1), SEQ ID NO: 6038 (700458240H1), SEQ ID NO: 4564 (700455257H1), SEQ ID NO: 5895 (700457929H1), SEQ ID NO: 5646 (700457402H1), SEQ ID NO: 4666 (700455575H1), SEQ ID NO: 4665 (700455574H1), SEQ ID NO: 3931 (700454106H1), SEQ ID NO: 4089 (700454406H1), SEQ ID NO: 3953 (700454139H1), SEQ ID NO: 3932 (700454107H1), SEQ ID NO: 3901 (700454033H1), SEQ ID NO: 3890 (700453995H1), SEQ ID NO: 5558 (700457247H1), SEQ ID NO: 4863 (700455922H1), SEQ ID NO: 3841 (700453912H1), SEQ ID NO: 6215 (700458576H1), SEQ ID NO: 4495 (700455139H1), SEQ ID NO: 5560 (700457250H1), SEQ ID NO: 3989 (700454219H1), SEQ ID NO: 6114 (700458404H1), SEQ ID NO: 5189 (700456528H1), SEQ ID NO: 5722 (700457568H1), SEQ ID NO: 4563 (700455255H1), SEQ ID NO: 6040 (700458242H1), SEQ ID NO: 5547 (700457230H1), SEQ ID NO: 4104 (700454426H1), SEQ ID NO: 4681 (700455604H1), SEQ ID NO: 6322 (700458877H1), SEQ ID NO: 3608 (700405305H1), SEQ ID NO: 4261 (700454705H1), SEQ ID NO: 5308 (700456783H1), SEQ ID NO: 5178 (700456503H1), SEQ ID NO: 3758 (700453762H1), SEQ ID NO: 3756 (700453759H1), SEQ ID NO: 3708 (700405463H1), SEQ ID NO: 5243 (700456663H1), SEQ ID NO: 6202 (700458557H1), SEQ ID NO: 6196 (700458549H1), SEQ ID NO: 5339 (700456844H1), SEQ ID NO: 4568 (700455263H1), SEQ ID NO: 5608 (700457336H1), SEQ ID NO: 4939 (700456049H1), SEQ ID NO: 5546 (700457225H1), SEQ ID NO: 5416 (700457006H1), SEQ ID NO: 5911 (700457961H1), SEQ ID NO: 5007 (700456175H1), SEQ ID NO: 5564 (700457254H1), SEQ ID NO: 5565 (700457255H1), SEQ ID NO: 5848 (700457848H1), SEQ ID NO: 4670 (700455579H1), SEQ ID NO: 5388 (700456942H1), SEQ ID NO: 4820 (700455846H1), SEQ ID NO: 5806 (700457760H1), SEQ ID NO: 5873 (700457888H1), SEQ ID NO: 5955 (700458063H1), SEQ ID NO: 5161 (700456463H1), SEQ ID NO: 4117 (700454445H1), SEQ ID NO: 5475 (700457093H1), SEQ ID NO: 3835 (700453894H1), SEQ ID NO: 3881 (700453979H1), SEQ ID NO: 5289 (700456751H1), SEQ ID NO: 4687 (700455611H1), SEQ ID NO: 5778 (700457688H1), SEQ ID NO: 5663 (700457434H1), SEQ ID NO: 6140 (700458451H1), SEQ ID NO: 4441 (700455046H1), SEQ ID NO: 4824 (700455853H1), SEQ ID NO: 3827 (700453878H1), SEQ ID NO: 4130 (700454464H1), SEQ ID NO: 4110 (700454435H1), SEQ ID NO: 5793 (700457726H1), SEQ ID NO: 5506 (700457147H1), SEQ ID NO: 6164 (700458509H1), SEQ ID NO: 4048 (700454329H1), SEQ ID NO: 5424 (700457020H1), SEQ ID NO: 5420 (700457012H1), SEQ ID NO: 6151 (700458468H1), SEQ ID NO: 4947 (700456059H1), SEQ ID NO: 4352 (700454902H1), SEQ ID NO: 6111 (700458395H1), SEQ ID NO: 6220 (700458587H1), SEQ ID NO: 6234 (700458616H1), SEQ ID NO: 4560 (700455252H1), SEQ ID NO: 4392 (700454967H1), SEQ ID NO: 4881 (700455944H1), SEQ ID NO: 4929 (700456036H1), SEQ ID NO: 5568 (700457258H1), SEQ ID NO: 5570 (700457260H1), SEQ ID NO: 5545 (700457223H1), SEQ ID NO: 4523 (700455179H1), SEQ ID NO: 5750 (700457637H1), SEQ ID NO: 4873 (700455936H1), SEQ ID NO: 6186 (700458537H1), SEQ ID NO: 6198 (700458552H1), SEQ ID NO: 5414 (700456994H1), SEQ ID NO: 5799 (700457740H1), SEQ ID NO: 5669 (700457445H1), SEQ ID NO: 5754 (700457644H1), SEQ ID NO: 4640 (700455518H1), SEQ ID NO: 3785 (700453807H1), SEQ ID NO: 6245 (700458639H1), SEQ ID NO: 3844 (700453919H1), SEQ ID NO: 3682 (700405418H1), SEQ ID NO: 3627 (700405340H1), SEQ ID NO: 5005 (700456169H1), SEQ ID NO: 4948 (700456061H1), SEQ ID NO: 4355 (700454907H1), SEQ ID NO: 4894 (700455964H1), SEQ ID NO: 5485 (700457120H1), SEQ ID NO: 4586 (700455286H1), SEQ ID NO: 4410 (700455001H1), SEQ ID NO: 4375 (700454941H1), SEQ ID NO: 4251 (700454684H1), SEQ ID NO: 6278 (700458802H1), SEQ ID NO: 4880 (700455943H1), SEQ ID NO: 5092 (700456346H1), SEQ ID NO: 4830 (700455863H1), SEQ ID NO: 4827 (700455858H1), SEQ ID NO: 5219 (700456619H1), SEQ ID NO: 5629 (700457371H1), SEQ ID NO: 5404 (700456971H1), SEQ ID NO: 4525 (700455183H1), SEQ ID NO: 4312 (700454820H1), SEQ ID NO: 3860 (700453944H1), SEQ ID NO: 5676 (700457457H1), SEQ ID NO: 5673 (700457451H1), SEQ ID NO: 5915 (700457969H1), SEQ ID NO: 5108 (700456369H1), SEQ ID NO: 4115 (700454443H1), SEQ ID NO: 4518 (700455174H1), SEQ ID NO: 4359 (700454913H1), SEQ ID NO: 5493 (700457130H1), SEQ ID NO: 4057 (700454344H1), SEQ ID NO: 4975 (700456105H1), SEQ ID NO: 3803 (700453835H1), SEQ ID NO: 3799 (700453827H1), SEQ ID NO: 4953 (700456066H1), SEQ ID NO: 5672 (700457449H1), SEQ ID NO: 4290 (700454767H1), SEQ ID NO: 3742 (700453733H1), SEQ ID NO: 6290 (700458825H1), SEQ ID NO: 6248 (700458643H1).

4. A nucleic acid sequence complementary to at least a fragment of at least one corn seedling-derived polynucleotide of claim 1.

5. A method for detecting a polynucleotide in a biological sample, the method comprising the steps of:
a) providing a biological sample containing nucleic acids,
b) hybridizing a polynucleotide of claim 4 to nucleic acids in the biological sample, thereby forming a hybridization complex; and
c) detecting the hybridization complex, wherein the presence of the hybridization complex correlates with the presence of the polynucleotide in the sample.

6. The method of claim 5 wherein the nucleic acids of the biological sample are amplified prior to hybridization.

7. A method for using oligomers and amplification to recover a regulatory element from a DNA library, the method comprising the steps of:
a) designing oligomers to at least one of the sequences of claim 1;
b) combining the oligomers with a DNA library under appropriate conditions to amplify the sequence of step a);
c) comparing the nucleic acid sequence of step a) with the amplified sequence to identify overlapping areas;
d) identifying additional sequence beyond the overlapping areas, and
e) repeating steps a) through d) until the regulatory element is recovered.

8. A seedling-specific regulatory element which regulates the expression of at least one of the sequences of claim 1.

9. An expression vector containing the regulatory element of claim 8.

10. A plant containing the expression vector of claim 9.

11. An expression vector containing the corn seedling-derived polynucleotide of claim 1.

12. A host cell containing the expression vector of claim 11.

13. A method for producing a corn seedling-derived polypeptide, said method comprising the steps of:
a) culturing the host cells of claim 12 under conditions suitable for the expression of the corn seedling-derived polypeptide; and
b) recovering the corn seedling-derived polypeptide from the cell culture.

14. A purified corn seedling-derived polypeptide (CDP) encoded by at least one of the corn seedling-derived polynucleotides of claim 1.

15. An anti-CDP antibody specifically binding the purified corn seedling-derived polypeptide of claim 14.

16. A method of identifying a test compound which specifically binds to the polypeptide of claim 14, the method comprising the steps of:
a) providing a test compound;
b) combining the polypeptide with the test compound for a sufficient time and under suitable conditions for binding; and
c) detecting binding of the polypeptide to the test compound, thereby identifying the compound which specifically binds the polypeptide.

17. A method of screening a plurality of molecules for specific binding to the polynucleotide of claim 1, the method comprising the steps of:
a) providing the plurality of molecules;
b) combining the polynucleotide with each of the plurality of molecules for a time sufficient to allow binding under suitable conditions; and
c) detecting binding of the polynucleotide to each of the plurality of molecules, thereby identifying the molecules which specifically bind the polynucleotide.
